(19) United States
(12) Patent Application Publication
(10) Pub. No.: US 2023/0210902 A1
(43) Pub. Date: Jul. 6, 2023

(54) SARS-COV-2-SPECIFIC T CELLS

(71) Applicant: BOARD OF REGENTS, THE UNIVERSITY OF TEXAS SYSTEM, Austin, TX (US)

(72) Inventors: Katy REZVANI, Houston, TX (US); Elizabeth SHPALL, Houston, TX (US); Rafet BASAR, Houston, TX (US); David MARIN COSTA, Houston, TX (US); Nadima UPRETY, Houston, TX (US); Emily ENSLEY, Houston, TX (US)

(21) Appl. No.: 17/997,148

(22) PCT Filed: May 11, 2021
(86) PCT No.: PCT/US2021/070532

§ 371 Date: Oct. 26, 2022

Related U.S. Application Data
(60) Provisional application No. 63/076,842, filed on Sep. 10, 2020.
(60) Provisional application No. 63/022,896, filed on May 11, 2020.

(51) Int. Cl. A61P 31/14; A61K 45/06; C07K 16/10; A61K 35/17; C12N 5/0783; C12N 15/11; C12N 9/22; C12N 15/90

(52) U.S. Cl. CPC A61K 35/17; A61K 45/06; A61P 31/14; C07K 16/10; C12N 5/0638; C12N 9/22; C12N 15/11; C12N 15/907; C12N 2310/20; C12N 2501/998; C12N 2501/2302; C12N 2501/2304; C12N 2501/2307; C12N 2501/2315; C12N 2501/2321; C12N 2800/80

(57) ABSTRACT

Embodiments of the disclosure includes methods of producing viral-specific therapy (VST) cells specific for the SARS-CoV-2 virus and uses of the cells. The methods may utilized peptide mixtures and stimulation of mononuclear cells using particular cytokine cocktails. The cells may also be genetically modified to lack expression of one or more endogenous genes, including one or more genes that renders the cells more effective and/or able to withstand deleterious conditions, such as the presence of glucocorticoids.

### CROSS-REFERENCE TO RELATED APPLICATIONS

This application claims priority to U.S. Provisional Pat. Application Serial No. 63/022,896, filed May 11, 2020, and also claims priority to U.S. Provisional Pat. Application Serial No. 63/076,842, filed Sep. 10, 2020, both of which applications are incorporated by reference herein in their entirety.

### STATEMANT REGARDING FEDERALLY SPONSORED

### RESEARCH OR DEVELOPMENT

This invention was made with government support under grant number CA211044 awarded by the National Institutes of Health. The government has certain rights in the invention.

### TECHNICAL FIELD

Embodiments of the disclosure encompass at least the fields of cell biology, molecular biology, cell therapy, virology, immunotherapy, and medicine.

### BACKGROUND

The emergence of severe acute respiratory syndrome coronavirus 2 (SARS-CoV-2, also referred to as COVID-19) in 2019, nearly two decades after the first SARS-CoV, and almost 10 years after the Middle East respiratory syndrome coronavirus (MERS-CoV), marks the third large-scale epidemic of coronavirus in the human population in recent times. Unfortunately, however, there are no approved preventive or therapeutic agents for these highly pathogenic viruses.

SARS-CoV, MERS and COVID-19 (SARS-CoV-2) manifest as severe atypical pneumonia associated with high morbidity and mortality in humans. A dysregulated/excessive innate response associated with T-cell lymphopenia are leading contributors to the lung pathology in this syndrome (Kong et al., 2009). Decreased numbers of T cells correlate with the severity of acute phase of SARS disease in humans and delayed clearance of the virus (Thevarajan et al., 2020). Several lines of evidence from other respiratory virus infections such as influenza A and para-influenza have established that virus-specific CD4 and CD8 T cells generated during primary and memory response can clear the virus and protect the host from severe infections (Christensen et al., 2000; Eichelberger et al., 1991). Indeed, studies from SARS and MERS-infected patients and animals also point to an important role for the T cell-mediated adaptive immune response in protection and clearance of respiratory coronavirus infection (Park et al., 2020). In SARS-recovered patients, neutralizing antibody titers and the memory B cell response are short-lived and only last a few months, whereas SARS-CoV-specific memory T cells persist for up to 6 years post-infection (Libraty et al., 2007).

Direct evidence for the role of virus-specific CD4 and CD8 T cells in SARS-CoV clearance and host protection come from adoptive transfer studies. Adoptively infused SARS-CoV-specific effector CD4 and CD8 T cells in mice susceptible to SARS resulted in rapid virus clearance and improvement in the clinical disease and survival of the animals (Zhao et al., 2010).

The present disclosure provides solutions to needs in the art of coronavirus treatment and prevention.

### BRIEF SUMMARY

The present invention is directed to methods and compositions for treatment or prevention of coronavirus infection, including at least of SARS-CoV-2. In particular embodiments, T cells specific for SARS-CoV-2 antigens are utilized to treat or prevent SARS-CoV-2 infection in an individual that has the infection or that is at high risk for the infection, in at least some cases. The cells may delay onset and/or severity of infection. The produced cells may be allogeneic or autologous with respect to a recipient individual. In particular embodiments, the disclosure encompasses off-the-shelf, third party SARS-CoV-2-specific T cells for infected individuals. In some cases, the cells for therapy are also modified in another manner, such as (1) having knockout or knock down of one or more genes endogenous to the cells; and/or (2) incorporating one or more engineered antigen receptors in the cells. An example of an endogenous gene for genetic modification is the glucocorticoid receptor that when knocked prevents cytolysis of the cells in the presence of one or more glucocortocoids as treatment for the individual. In specific embodiments, the cells are partially HLA-matched with respect to a recipient individual.

Embodiments of the disclosure provide ex vivo methods of preparing SARS-CoV-2-specific T cells, comprising the step of culturing a starting population of cells in the presence of a mixture of peptides and one or more of IL-2, IL-4, IL-7, IL-15, and IL-21, wherein the mixture of peptides comprises overlapping peptides spanning at least one protein from SARS-CoV-2, thereby producing SARS-CoV-2-specific T cells. The SARS-CoV-2-specific T cells may be further defined as cytotoxic T cells (CTLs). The starting population of cells comprises peripheral blood mononuclear cells (PBMCs), lymphocytes, or a mixture thereof. The starting population may be derived from one or more healthy donors, one or more individuals that will receive the cells, one or more asymptomatic SARS-CoV-2-positive individuals, one or more SARS-CoV-2-negative individuals, one or more individuals that were SARS-CoV-2-positive followed by being SARS-CoV-2-negative, one or more individuals that have antibodies to SARS-CoV-2, one or more individuals that lack antibodies to SARS-CoV-2, one or more individuals that have been vaccinated by any SARS-CoV-2 vaccine, or a combination thereof. In some cases, the mononuclear cells were obtained from blood, buffy coat, or both. The culturing may be for 7-14 days or any subrange therebetween, including for 10-14 days. In some cases, the culturing does not comprise a second population of cells pre-stimulated with the mixture of peptides. The pre-stimulated cells may be further defined as antigen presenting cells (APCs), such as dendritic cells, monocytes, and/or B lymphocytes.

Peptides used in the disclosure may have a length of 8-30 amino acids, or any subrange therebetween, including having a length of 12-18 amino acids; the peptides may be 15 amino acids in length. In some cases, the peptides in the mixture of peptides overlap by 10-15 contiguous amino acids from a corresponding protein in SARS-CoV-2. The peptides in the mixture may be 15 amino acids in length and overlap by 11 continuous amino acids from a corresponding protein in SARS-CoV-2. In some cases, the mixture of peptides comprises peptides that span part or all of the entire length of one or more (or two or more) proteins of SARS-CoV-2.

In some embodiments, the methods further comprise the step of genetically modifying the starting population of cells or the SARS-CoV-2-specific T cells to (a) disrupt expression of one or more endogenous genes in the cells; and/or (b) express one or more chimeric antigen receptors (CAR) and/or one or more T cell receptors (TCR). The endogenous gene may be one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, TDAG8, Cbl-b, B2M, HLA class I, and CD7. In specific embodiments, the genetically modifying occurs by CRISPR. In some cases, the T cells are genetically modified to express a CAR and/or an engineered TCR, and they may target one or more SARS-CoV-2 antigens.

Populations of SARS-CoV-2-specific T cells, produced by any one of the methods herein, are encompass herein and may be comprised in a pharmaceutically acceptable carrier. Compositions comprising the population are contemplated.

In one embodiment, there is a method of treating or preventing a SARS-CoV-2 infection in an individual, comprising the step of delivering to the individual an effective amount of populations or compositions of the disclosure to the individual. The population or composition may be administered by infusion, intravenously, intraperitoneally, intratracheally, intramuscularly, endoscopically, intralesionally, percutaneously, subcutaneously, regionally, intracranially, by direct injection, or by perfusion. In some cases, the individual has SARS-CoV-2 infection or is at high risk for having SARS-CoV-2 infection (such as being elderly (e.g., 65 years of age or older), overweight or obese, having diabetes, heart disease, having cancer, chronic kidney disease, COPD, asthma, pulmonary hypertension, heart disease, liver disease, HIV, is a smoker, has received stem cell or organ transplant, or being immunocomprised). The individual does not have SARS-CoV-2 infection or has tested negative for SARS-CoV-2 infection, in some cases. The cells may be autologous or allogeneic with respect to the individual. Any population or composition may be delivered by infusion. In some cases, the individual has acute respiratory distress syndrome or pneumonia.

In specific embodiments, the SARS-CoV-2-specific T cells have been genetically modified to lack expression of or have reduced expression of NR3C1, and in some cases the individual has received, is receiving, and/or will receive one or more glucocorticoids, such as beclomethasone, betamethasone, budesonide cortisone, dexamethasone, hydrocortisone, methylprednisolone, prednisolone, prednisone, triamcinolone, or a combination thereof.

The individual may be administered an effective amount of one or more additional therapies, such as Azithromycin, AC-55541, Apicidin, AZ3451, AZ8838, Bafilomycin A1, CCT 365623, Daunorubicin, E-52862, Entacapone, GB110, H-89, Haloperidol, Indomethacin, JQ1, Loratadine, Merimepodib, Metformin, Midostaurin, Migalastat, Mycophenolic acid, PB28, PD-144418, Ponatinib, Ribavirin, RS-PPCC, Ruxolitinib, RVX-208, S-verapamil, Silmitasertib , TMCB, UCPH-101, Valproic Acid, XL413, ZINC1775962367, ZINC4326719, ZINC4511851, ZINC95559591, 4E2RCat, ABBV-744, Camostat, Captopril, CB5083, Chloramphenicol, Chloroquine (and/or Hydroxychloroquine), CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/M1N128), FK-506 (Tacrolimus), Ternatin 4 (DA3), Tigecycline, Tomivosertib (eFT-508), Verdinexor, WDB002, Zotatifin (eFT226), or a combination thereof. In some embodiments, the individual is additionally or alternatively administered an effective amount of SARS-CoV-2-specific NK cells. In specific cases, the NK cells comprise: (a) one or more engineered antigen receptors that target one or more proteins from the SARS-CoV-2 virus; (b) one or more engineered antigen receptors that target a receptor used by SARS-CoV-2 to enter a host cell; and/or (c) one or more engineered antigen receptors that target a ligand on the surface of a SARS-CoV-2-infected cell. The engineered antigen receptor may be a chimeric antigen receptor (CAR), an engineered T cell receptor (TCR), or both. In specific cases, the engineered antigen receptor of (a) comprises at least one scFv that targets a protein from the SARS-CoV-2 virus. The engineered antigen receptor of (a) may target the spike protein, membrane protein, envelope protein, nucleocapsid protein, Nsp2, Nsp3, Nsp4, Nsp6, Nsp7, Nsp8, Nsp9, Nsp10, Nsp11, 3C-like proteinase, leader protein, ORF7b, 2′-O-ribose methyltransferase, endoRNAse, 3′-to-5′ exonuclease, helicase, RNA-dependent RNA polymerase, orfla polyprotein, ORF10 protein, ORF8 protein, ORF7a protein, ORF6 protein, ORF3a, or orflab polyprotein. In specific embodiments, the engineered antigen receptor of (b) targets angiotensin-converting enzyme-2 (ACE2) on a host cell. The engineered antigen receptor of (c) may target a ligand of Natural killer group 2D receptor (NKG2DR), such as MHC class I chain-related protein (MIC)A, MICB, UL16 binding protein 1 (ULBP), or Poliovirus Receptor (PVR). One or more endogenous genes in the NK cell have been reduced or eliminated in expression, such as NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, Cbl-b, B2M, HLA class I, or a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings (also “Figure” and “FIG.” herein), of which:

FIG. 1 shows one example of a procedure to generate SARS-CoV-2-specific cells.

FIG. 2 shows the ‘backgound’ intracellular interferon-gamma staining by T-cells in the absence of stimulation with pepmix (negative control- left panel) and in response to stimulation with phorbol myristate acetate (PMA) and inomycin (positive control).

FIG. 3 demonstrates for Donor 1- interferon (IFN) gamma response to stimulation with COVID 19 antigen- briefly, PBMCs were stimulated with pepmixes derived from the M, N and S proteins of COVID19, either individually or in combination, and cultured in different cytokine cocktails. After 14 days of expansion, over 17% of CD4+ T cells are directed against S protein and over 5% against M protein.

FIG. 4 demonstrates for Donor 1- TNF-alpha response to stimulation with COVID 19 antigen- briefly, PBMC were stimulated with pepmixes derived from the M, N and S proteins of COVID19, either individually or in combination, and cultured in different cytokine cocktails- After 14 days of expansion, 17% of CD4+ T cells are directed against S protein and nearly 4% against M protein.

FIG. 5 shows for Donor 1- IL-2 response to stimulation with COVID 19 antigen-briefly, PBMC were stimulated with pepmixes derived from the M, N and S proteins of COVID19, either individually or in combination, and cultured in different cytokine cocktails. After 14 days of expansion, nearly 2% of CD4+ T cells are directed against S protein and over 3% against M protein.

FIGS. 6A and 6B show for Donor 2-IFN gamma and TNF-alpha response to stimulation with COVID 19 antigen- briefly,PBMC were stimulated with pepmixes derived from the M, N and S proteins of COVID19, either individually or in combination, and cultured in different cytokine cocktails. After 14 days of expansion, nearly 8% of CD4+ T cells are directed against the M protein and nearly 5% against the N protein. FIG. 6A shows culture with IL-2, IL-4, and IL-7. FIG. 6B shows culture with IL-2, IL-7, and IL-15.

FIGS. 7A-7G. show successful expansion of COVID-19 reactive T cells from COVID-19 recovered donors. FIG. 7A, Bar graph showing the log 10 fold expansion of COVID-19 reactive T cells cultured with different cytokine cocktails, IL-2/4/7 (left), IL-2/7/15 (second from left), IL-2/4/21 (second from right) and IL-2/7/21 (right). FIG. 7B, Bar graphs showing the log 10 fold expansion of the CD4+ (left) and CD8+ (right) subsets of COVID-19 reactive T cells cultured under the different cytokine stimulation conditions. FIG. 7C, Quantification of EM (CD45RO+ CD45RA- CD62L-, left panel), CM (CD45RO+ CD45RA- CD62+, middle panel) and EMRA (CD45RO- CD45RA+ CD62L-, right panel) in COVID-19 reactive T cells expanded with IL-2/4/7 (left) or IL-2/7/15 (right) (n=8 samples per group); bars represent median values with interquartile range. p-values are indicated at the top of each graph. FIG. 7D, Mass cytometry analysis of T cells (gated on CD45+CD3+) expanded from 8 recovered donors using combination of M, N and S peptide libraries and cytokine cocktails (IL-2/4/7 and IL-27/15 conditions are overlapped in this phenograph). tSNE map shows the 32 clusters obtained, each highlighted in corresponding color. Cluster 32 (circled) represents the polyfunctional COVID-19 reactive T cells. FIG. 7E, Individual tSNE maps showing the expression of IFNγ,TNFα and MIP1β mostly restricted to cluster 32. Expression levels are indicated by color scale, ranging from blue representing low expression to red representing high expression. FIG. 7F, Cluster identity and frequency are summarized in heatmaps showing marker expression levels (X axis) for T cell populations (Y axis) expanded with the two different cytokine cocktails IL-2/4/7 (left heatmap) or IL-2/7/15 (right heatmap). Markers associated with function, phenotype, activation or exhaustion are indicated below each heatmap. Expression level is indicated by color scale, ranging from low (blue) to high (red). Cluster 32 is indicated with a rectangle. FIG. 7G, Violin plots comparing expression of TIM3, LAG3, TIGIT, PD1, and CTLA4 and between the two cytokine stimulation conditions IL-2/4/7 (blue) and IL-2/7/15 (red), n = 8. p-values are indicated at the top of each graph.

FIGS. 8A-8C show that expanded COVID-19 CTLs are directed against structural proteins including the C and N terminals of the S protein. FIG. 8A, Bar graphs showing the percentage of total IFNy (+) COVID-19 reactive CD3+ T cells stimulated with the peptide libraries derived from the different structural proteins M (left), N (middle), S (right) cultured with different cytokine cocktails IL-2/4/7 (left panel) or IL-2/7/15 (right panel). FIG. 8B, Bar graphs showing IFNγ (+) expression in CD4+ and CD8+ T cell subsets of COVID-19 reactive T cells stimulated with the peptide libraries derived from the different structural proteins M (blue), N (red), S (green) cultured with IL-2/4/7 (left panels) or IL-2/7/15 (right panels). FIG. 8C, Quantification of IFNγ (+) COVID-19 reactive T cells subsets (CD4+ or CD8+) directed against N-terminus (S1, bars on the left in the pairs) or the C-terminus (S2, bars on the right in the pairs) of the S protein in both IL-2/4/7 (left panels) and IL-2/7/15 (right panels) stimulation conditions (n=8 samples per group). Bars represent median values with interquartile range. p-values are indicated at the top of each graph.

FIGS. 9A-9D. Pattern of antigenic responses after expansion of COVID-19 reactive T cells from COVID-19 recovered donors compared to baseline. FIG. 9A, Bar graph showing the percentage of CD4+ and CD8+ subsets of COVID-19 reactive T cells at baseline. FIG. 9B, Graphical analysis showing the percentage of IFNγ (+) COVID-19 reactive T cells from recovered donors at baseline in the CD4+ compartment (left panel) or CD8+ compartment (right panel) when stimulated with the peptide libraries derived from the different structural proteins M (left), N (middle), S (right). FIG. 9C, FIG. 9D Pie charts illustrating the percent distribution of IFNγ (+) CD4 and CD8 T cells reactive to M, N or S1, S2 peptide libraries at baseline (FIG. 9C) or following expansion with IL-2/4/7 (upper panel) or IL-2/7/15 (lower panel) cytokine cocktails (FIG. 9D).

FIGS. 10A-10B show that COVID-19 CTLs can be expanded from the PB of healthy donors but at lower frequencies compared to recovered donors. FIG. 10A, Graphical representation of the log 10 fold expansion of COVID-19 reactive T cells derived from healthy donors cultured with different cytokine cocktails, IL-2/4/7 (left), IL-2/7/15 (right). FIG. 10B, Comparison of COVID-19 reactive T cell expansion between recovered donors (RD, left) and healthy donors (HD, right) cultured under the different cytokine stimulation conditions IL-2/4/7 (left panel) and IL-2/7/15 (right panel).

FIGS. 11A-11G show that expanded COVID-19 CTLs can be genetically modified to be rendered become steroid resistant. A,B NR3C1 KO efficiency shown by PCR gel electrophoresis (FIG. 11A) and by western blot (FIG. 11B) in COVID-19 CTLs expanded with IL-2/4/7 or IL-2/7/15, after electroporation with Cas9 complexed with crRNA 1 and crRNA 2 targeting exon 2 of the NR3C1 gene. COVID-19 CTLs electroporated with Cas9 alone were used as controls. β-actin was used as loading control for western blot. FIG. 11C, Representative FACS plots showing the percentage of apoptotic cells (annexin V+) and live or dead cells (live/dead stain) in control Cas9 vs NR3C1 KO COVID-19 CTLs after culture with or without dexamethasone (Dex; 200 µM) for 72 hours. Inset values indicate the percentage of annexin V and alive/dead cells from each group. FIG. 11D, Bar graph summarizing the percentage of live cells between control Cas9 and NR3C1 KO COVID-19 CTLs treated with or without 200 µM dexamethasone for 72 hours (n = 3). Bars represent median values with interquartile range. p-values are indicated above the graphs, and from left to right the bars represent Cas9, Cas9+Dex200 µM, NR3C1 KO, and NR3C1 KO + Dex 200 µM. FIG. 11E, Representative FACs plots showing the distribution of CD4+ and CD8+ T cells (upper panel) and phenotype based on CD62L and CD45RA expression (lower panel) in Cas9 alone or NR3C1 KO COVID-19 CTLs with or without 200 µM dexamethasone. FIG. 11F, Percentage of CD4+ and CD8+ T cells within COVID-19 CTLs treated with control Cas9 (left), NR3C1 KO (middle), or NR3C1 KO plus dexamethasone (Dex; 200 µM; right). FIG. 11G, Frequency of COVID-19 CTLs producing IFNγ, TNFα, or IL-2 control Cas9 (left), NR3C1 KO (middle), or NR3C1 KO plus dexamethasone (Dex; 200 µM; right) in response to 6 hours of stimulation with viral PepMix (n = 3). The functional analysis of the Cas9+Dex group was not performed due to the absence of viable cells resulting from the lymphocytotoxic effect of steroids. The bars represent mean values with SD. NS, not significant.

FIG. 12 demonstrates that a cytokine profile from COVID-19 reactive T cells supports a functional profile without CRS. Multiplex cytokine analysis showing the concentration of different cytokines in pg/ml detected in supernatants from COVID-19 reactive T cells stimulated with the different peptide libraries (S1, S2, M and N, either separately or in combination) at baseline (green triangles) or after expansion with IL-2/4/7 (blue circles) or IL-2/7/15 (red squares), (n=4 samples per group).

FIGS. 13A-13B show that expanded COVID-19 CTLs are directed against structural proteins, including both the C and N terminals of the S protein. FIG. 13A, Percentage of IFNγ(+) COVID-19 reactive CD3+ T cells stimulated with the peptide libraries derived from the different structural proteins M (left), N (middle), S (right) cultured with different cytokine cocktails IL2/4/21 (left panel) or IL2/7/21 (right panel). FIG. 13B, Quantification of IFNγ (+) COVID-19 reactive T cells subsets (CD4+ or CD8+) directed against N-terminus (S1, left) or the C-terminus (S2, right) of the S protein in both IL-2/4/21 (left panels) and IL-2/7/21 (right panels) stimulation conditions (n=7 samples per group). Bars represent median values with interquartile range. p-values are indicated at the top of each graph. Corresponding pie charts showing the percent distribution of M, N, S1 and S2 reactive IFNγ (+) T cells are depicted under each bar graph. A pie chart was not generated for the CD8+ T cells cultured with IL-2/4/21 due to the very low number of cells.

FIG. 14 demonstrates that an absolute number of COVID-19 specific T cells cultured with IL-2/4/7 correlates with antibody titer of Spike protein IgG. Scatter plots showing correlation between absolute number of COVID-19 specific T cells (on Y axis) and antibody titers on the X axis (Spike IgG) of COVID-19 specific T cells expanded with IL-2/4/7 (left panel) or IL-2/7/15 (right panel).

FIGS. 15A-15C demonstrate that COVID-19 CTLs can be expanded from the PB of healthy donors but at lower frequencies compared to COVID-19-recovered donors. FIG. 15A, FIG. 15B Graphical analysis of IFNy (+) COVID-19 reactive CD3+ T cells from healthy donors stimulated with the peptide libraries derived from the different structural proteins M, N, S at (FIG. 15A) baseline or (FIG. 15B) cultured with IL-2/4/7 (left panel) or IL-2/7/15 (right panel). Corresponding pie charts show the distribution of M, N, or S reactive IFNy (+) T cells for each group (n=5 samples per group). Bars represent median values with interquartile range. p-values are indicated at the top of each graph. FIG. 15C, Percentage of IFNγ (+) COVID-19 reactive CD4 and CD8 T cells generated from PB of healthy donors stimulated with the peptide libraries derived from the different structural proteins M, N, S after expansion with IL-2/4/7 (left panels) or IL-2/7/15 (right panels). Distribution of M, N or S reactive IFNγ (+) T cells for each group is shown in pie charts below corresponding graphs (n=5 samples per group). Bars represent median values with interquartile range. p-values are indicated at the top of each graph.

FIGS. 16A-16D show that CD4 CTLs produce cytokines in response to exposure to COVID19 variants. SARS-CoV-2 CTLs were generated that target membrane, nucleocapsid and spike proteins, as described in the Examples. Intracellular staining was performed to assess TNFα, IFNγ, IL-2 and Granzyme B production of COVID-19 specific T cells that were exposed to spike peptides from the original strain (represented by S1 and S2 pools) or from certain emerging variants (B117, B1351, B11248, and B11249). Negative controls were PBMC without stimulation with pepmix. Bar graphs represent the percentage of TNFα (FIG. 16A), IFNγ (FIG. 16B), IL-2 (FIG. 16C) and granzyme B (FIG. 16D) produced by the CD4+ fraction of the COVID-19 specific T cells.

FIGS. 17A-17D demonstrate that CD8 CTLs produce cytokines in response to exposure to COVID19 variants. Sars-Cov2 CTLs were generated by first incubating PBMC withlug of membrane, nucleocapsid and spike pepmixes for 2 hours, then expanded in complete Click’s media supplemented with IL-2 (20 IU/ml), IL-4 (60 ng/ml) and IL-7 (10 ng/ml) for 14 days to generate COVID-19 specific T cells. Intracellular staining was performed to assess TNFα, IFNγ, IL-2 and Granzyme B production of COVID-19 specific T cells that were exposed to spike peptides (1ug) from the original strain or from the emerging variants (B117, B1351, B11248, and B11249) for 6 hours. Bar graphs represent the percentage of TNFα (FIG. 17A), IFNγ (FIG. 17B), IL-2 (FIG. 17C), and granzyme B (FIG. 17D) produced by the CD8+ fraction of the COVID-19 specific T cells.

### DETAILED DESCRIPTION

This application incorporates by reference herein in its entirety a patent application filed on the same day and entitled “NATURAL KILLER CELL IMMUNOTHERAPY FOR THE TREATMENT OR PREVENTION OF SARS-CoV-2 INFECTION.”

### I. Examples of Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. In specific embodiments, aspects of the disclosure may “consist essentially of” or “consist of” one or more sequences of the disclosure, for example. Some embodiments of the invention may consist of or consist essentially of one or more elements, method steps, and/or methods of the disclosure. It is contemplated that any method or composition described herein can be implemented with respect to any other method or composition described herein. The scope of the present application is not intended to be limited to the particular embodiments of the process, machine, manufacture, composition of matter, means, methods and steps described in the specification. As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring T cells, artificially produced T cells, or artificially modified T cells (e.g., fusion cells or genetically modified cells).

The term “T cell” refers to T lymphocytes, and includes, but is not limited to, γδ⁺ T cells, NK T cells, CD4⁺ T cells and CD8+ T cells. CD4⁺ T cells include T_{H0}, T_{H1} and T_{H2} cells, as well as regulatory T cells (T_{reg}). There are at least three types of regulatory T cells: CD4+CD25+T_{reg}, CD25 T_{H3} T_{reg}, and CD25 T_{R1} T_{reg}. “Cytotoxic T cell” refers to a T cell that can kill another cell. The majority of cytotoxic T cells are CD8+ MHC class I-restricted T cells, however some cytotoxic T cells are CD4+. In particular embodiments, the T cell of the present disclosure is CD4+ or CD8+.

A “leukocyte” refers to cells in the blood, also termed “white cells,” that are involved in defending a subject against infective organisms and foreign substances. Leukocytes are produced in the bone marrow. There are five main types, subdivided between two main groups: polymorphomnuclear leukocytes (neutrophils, eosinophils, basophils) and mononuclear leukocytes (monocytes and lymphocytes). Generally, when a subject has an infection, the production of leukocytes increases. A “lymphocyte: is a type of white blood cell involved in the immune defenses of the body. There are two main types of lymphocytes: B-cells and T-cells.

A “monocyte” is a large white blood cell in the blood that ingests microbes or other cells and foreign particles and proteins. When a monocyte passes out of the bloodstream and enters tissues, it develops into a macrophage.

As used herein, the term “antigen” is a molecule capable of being bound by an antibody or T-cell receptor. An antigen may generally be used to induce a humoral immune response and/or a cellular immune response leading to the production of B and/or T lymphocytes.

An “epitope” is the site on an antigen recognized by an antibody as determined by the specificity of the amino acid sequence. Two antibodies are said to bind to the same epitope if each competitively inhibits (blocks) binding of the other to the antigen as measured in a competitive binding assay. Alternatively, two antibodies have the same epitope if most amino acid mutations in the antigen that reduce or eliminate binding of one antibody reduce or eliminate binding of the other. Two antibodies are said to have overlapping epitopes if each partially inhibits binding of the other to the antigen, and/or if some amino acid mutations that reduce or eliminate binding of one antibody reduce or eliminate binding of the other.

The term “specifically binds” means to selectively bind with a single binding affinity for a particular antigen/epitope with which it immunoreacts. Examples include antigens and T cells that selectively immunoreact with a target antigen. In a particular example of specific binding, a T cell receptor on a target antigen-specific T cell specifically recognizes and reacts with a target antigen presented on an APC, such as an MHC complex, wherein the binding is a non-random binding reaction between the T cell receptor and a target antigenic determinant. In a specific example, the desired binding specificity of a target antigen-specific T cell is determined from the reference point of the ability of the T cell receptor on the target antigen-specific T cell to bind to an APC presenting the target antigen, but not an unrelated antigen, and therefore distinguish between two different antigens.

The term “antigen presenting cell (APC)” refers to a cell that carries on its surface major histocompatibility complex class I or class II molecules capable of presenting an antigen in the context of the MHC molecule to T cells. APCs include, but are not limited to, monocytes, macrophages, dendritic cells, B cells, and Langerhans cells.

The term “haplotyping or tissue typing” refers to a method used to identify the haplotype or tissue types of a subject, for example by determining which HLA locus (or loci) is expressed on the lymphocytes of a particular subject. The HLA genes are located in the major histocompatibility complex (MHC), a region on the short arm of chromosome 6, and are involved in cell-cell interaction, immune response, organ transplantation, development of cancer, and susceptibility to disease. There are six genetic loci important in transplantation, designated HLA-A, HLA-B, HLA-C, and HLA-DR, HLA-DP and HLA-DQ. At each locus, there can be any of several different alleles. A widely used method for haplotyping uses the polymerase chain reaction (PCR) to compare the DNA of the subject, with known segments of the genes encoding MHC antigens. The variability of these regions of the genes determines the tissue type or haplotype of the subject. Serologic methods are also used to detect serologically defined antigens on the surfaces of cells. HLA-A, --B, and -C determinants can be measured by known serologic techniques. Briefly, lymphocytes from the subject (isolated from fresh peripheral blood) are incubated with antisera that recognize all known HLA antigens. The cells are spread in a tray with microscopic wells containing various kinds of antisera. The cells are incubated for 30 minutes, followed by an additional 60-minute complement incubation. If the lymphocytes have on their surfaces antigens recognized by the antibodies in the antiserum, the lymphocytes are lysed. A dye can be added to show changes in the permeability of the cell membrane and cell death. The pattern of cells destroyed by lysis indicates the degree of histologic incompatibility. If, for example, the lymphocytes from a person being tested for HLA-A3 are destroyed in a well containing antisera for HLA-A3, the test is positive for this antigen group.

An “immune response” refers to a change in immunity, for example, a response of a cell of the immune system, such as a B cell, T cell, or monocyte, to a stimulus. In one example, the response is specific for a particular antigen (an “antigen-specific response”), such as a target antigen which has been selected for therapeutic purposes as a target of the immune response. In one example, an immune response is a T cell response, such as a CD4⁺ response or a CD8⁺ response. In another example, the response is a B cell response, and results in the production of specific antibodies. In a particular example, an increased or enhanced immune response is an increase in the ability of a subject to fight off a disease, such as a viral infection. Immune synapse: The region of association between an APC and an antigen-specific T cell. In a specific example, it is the complex formed between an antigen/WIC complex on an APC and the T cell receptor on the antigen-specific T cell.

The term “culturing” refers to the in vitro maintenance, differentiation, and/or propagation of cells in suitable media. By “enriched” is meant a composition comprising cells present in a greater percentage of total cells than is found in the tissues where they are present in an organism.

An “isolated” biological component (such as a portion of hematological material, such as blood components) refers to a component that has been substantially separated or purified away from other biological components of the organism in which the component naturally occurs. An isolated cell is one which has been substantially separated or purified away from other biological components of the organism in which the cell naturally occurs. For example, an isolated antigen-specific T cell population is a population of T cells that recognize a target antigen and which are substantially separated or purified away from other blood cells, such as other T cells.

The term “therapeutically effective amount” refers to an amount sufficient to produce a desired therapeutic result, for example an amount of purified target antigen-specific T cells sufficient to increase an immune response against the target antigen in a subject to whom the cells are administered. In particular examples, it is an amount effective to increase an immune response in a subject by at least 10%, for example at least 20%, at least 30%, at least 40%, at least 50%, or even at least 75%.

The term “subject” or “patient” or “individual” refer to either a human or non-human, such as primates, mammals, and vertebrates. In particular embodiments, the subject is a human. The subject is of any age, gender, or race.

The term “treatment” refers to a therapeutic intervention that ameliorates a sign or symptom of a disease or pathological condition, such a sign or symptom related to viral infection or prevention. In particular examples, treatment includes preventing a viral infection, for example by inhibiting the full development of a disease or condition associated with the virus. Prevention of a disease does not require a total absence of disease. For example, a decrease of at least 50% can be sufficient. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” or “effective” as used throughout this application refers to anything that promotes or enhances the well-being of the subject with respect to the medical treatment of this condition. This includes, but is not limited to, a reduction in the frequency or severity of the signs or symptoms of a viral infection and associated disease or medical condition.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer’s dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

A “peptide library,” “mixture of peptides” or “peptide mixture” are used interchangeably herein to refer to a plurality of peptides derived from coronavirus proteins including structural proteins spike (S), membrane (M), envelope (E) and nucleocapsid (N), as well as several proteins that are uncharacterized or non-structural proteins (all as examples only). The peptides may be overlapping at one or more amino acids of the protein sequence.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell, including T cells of any kind. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a viral antigen, for example. In some embodiments, CARs comprise an intracellular activation domain, a transmembrane domain, and an extracellular domain comprising a virus associated antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3zeta, FcR, CD27, CD28, CD137, DAP10, and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

Detailed herein are methods and compositions for coronavirus infection or prevention, in specific embodiments. The current coronavirus pandemic is unparalleled in world history with few therapeutic options currently available. SARS, MERS and COVID-19 manifest as severe atypical pneumonia associated with high morbidity and mortality in humans. The present disclosure provides novel methods and compositions at least for the generation of SARS-CoV-2-specific T cells for the treatment of patients with SARS-CoV-2 infection or at risk thereof.

### II. Coronavirus

The disclosure concerns methods and compositions for treatment or prevention of at least any virus referred to herein.

Coronaviridae is a family of enveloped, positive-sense, single-stranded RNA viruses. Coronavirus is the common name for Coronaviridae and Orthocoronavirinae (also referred to as Coronavirinae). The family Coronaviridae is organized in 2 sub-families, 5 genera, 23 sub-genera and about 40 species. They are enveloped viruses having a positive-sense single-stranded RNA genome and a nucleocapsid having helical symmetry. The genome size of coronaviruses ranges from about 26-32 kilobases.

The present disclosure encompasses treatment or prevention of infection of any virus in the Coronaviridae family. In certain embodiments, the disclosure encompasses treatment or prevention of infection of any virus in the subfamily Coronavirinae and including the four genera, Alpha-, Beta-, Gamma-, and Deltacoronavirus. In specific embodiments, the disclosure encompasses treatment or prevention of infection of any virus in the genus of Betacoronavirus, including the subgenus Sarbecovirus and including the species of severe acute respiratory syndrome-related coronavirus. In specific embodiments, the disclosure encompasses treatment or prevention of infection of any virus in the species of severe acute respiratory syndrome-related coronavirus, including the strains severe acute respiratory syndrome coronavirus (SARS-CoV) and severe acute respiratory syndrome coronavirus 2 (SARS-CoV-2, the virus that causes COVID-19). The disclosure encompasses treatment or prevention of infection any isolate, strain, type (including Type A, Type B and Type C; Forster et al., 2020, PNAS, https://doi.org/10.1073/pnas.2004999117), cluster, or sub-cluster of the species of severe acute respiratory syndrome-related coronavirus, including at least SARS-CoV-2. In specific embodiments, the virus being treated with methods and compositions of the disclosure is not SARS-CoV and is not MERS-CoV. In specific embodiments, the virus being treated with methods and compositions of the disclosure is SARS-CoV or is MERS-CoV. In specific embodiments, the virus has a genome length between about 29000 to about 30000, between about 29100 and 29900, between about 29200 and 29900, between about 29300 and 29900, between about 29400 and 29900, between about 29500 and 29900, between about 29600 and 29900, between about 29700 and 29900, between about 29800 and 29900, or between about 29780 and 29900 base pairs in length.

Examples of specific SARS-CoV-2 viruses include the following listed in the NCBI GenBank® Database, and these GenBank® Accession sequences are incorporated by reference herein in their entirety: (a) LC534419 and LC534418 and LC528233 and LC529905 (examples of different strains from Japan); (b) MT281577 and MT226610 and NC_045512 and MN996531 and MN908947 (examples of different strains from China); (c) MT281530 (Iran); (d) MT126808 (Brazil); (e) MT020781 (Finland); (f) MT093571 (Sweden); (g) MT263074 (Peru); (h) MT292582 and MT292581 and MT292580 and MT292579 (examples of different strains from Spain); (i) examples from the United States, such as MT276331 (TX); MT276330 (FL); MT276328 (OR) MT276327 (GA); MT276325 (WA); MT276324 (CA); MT276323 (RI); MT188341 (MN); and (j) MT276598 (Israel). In particular embodiments, the disclosure encompasses treatment or prevention of infection of any of these or similar viruses, including viruses whose genome has at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. In particular embodiments, the disclosure encompasses treatment or prevention of infection of any of these or similar viruses, including viruses whose genome has its entire sequence that is greater than 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. As one specific example, the present disclosure includes methods of treatment or prevention of infection of a virus having a genome sequence of SEQ ID NO:1 (represented by GenBank® Accession No. NC_045512; origin Wuhan, China) and any virus having a genome sequence with at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to SEQ ID NO:1. Infection with any strain of SARS-CoV-2 may be treated or prevented, including at least B.1.526, B.1.526.1, B.1.525, B.1.1.7, B.1.351, B.1.427, B.1.429, B.1.617, P.1, and P.2.

### III. Methods of Preparation of Cells

In particular embodiments, coronavirus-specific cells (hereafter referred to as SARS-CoV-2-specific T cells or COVID-19-specific T cells, and merely as examples) are prepared by one or more particular methods. In certain embodiments, the coronavirus-specific cells are produced upon stimulation of mononuclear cells with one or more peptide libraries spanning one or more viral antigens of as SARS-CoV-2. In particular cases, as SARS-CoV-2-specific T cells are prepared upon stimulating mononuclear cells (e.g., enriched from healthy donor or patient-derived blood or buffy coat) with one or more peptide libraries spanning one or more SARS-CoV-2 antigens. In specific embodiments, mononuclear cells enriched from healthy donor or patient-derived blood or buffy coat are stimulated with multiple different peptide libraries spanning multiple SARS-CoV-2 antigens.

The present disclosure encompasses adoptive immunotherapy using SARS-CoV-2-specific T cells generated ex vivo. In particular embodiments, delivery (such as by infusion) of SARS-CoV-2-specific T cells results in rapid viral clearance and improved outcomes in patients with SARS-CoV-2-related infections. In specific embodiments, delivery of HLA-matched or partially HLA-matched SARS-CoV-2-specific T cells generated from healthy donors results in rapid viral clearance and improved outcomes in patients with SARS-CoV-2-related infections. In some cases, the T cells are modified to lack expression of the glucocorticoid receptor and/or are modified to express an engineered antigen receptor.

The SARS-CoV-2-specific T cells in particular cases are prepared upon stimulating mononuclear cells with one or more peptide libraries spanning one or more SARS-CoV-2 antigens. Although peptides from any one or more of the proteins expressed from the SARS-CoV-2 virus may be utilized to produce the SARS-CoV-2-specific T cells, in specific cases, the peptide mix libraries are generated using one or more of the following 12 proteins: AP3A, NCAP, NS6, NS7A, NS7B, NS8, ORF10, ORF9B, Spike Glycoprotein, VEMP, VME1 and Y14 that encompassed the the proteins spike (S; for example, see GenBank® Accession No. YP_009724390); membrane (M; for example, see GenBank® Accession No. YP_009724393); envelope (E; for example, see GenBank® Accession No. YP_009724392); nucleocapsid (N; for example, see GenBank® Accession No. YP_009724397); Nsp2 (for example, see GenBank® Accession No. YP_009742609 or YP_009725298); Nsp3 (for example, see GenBank® Accession No. YP_009742610); Nsp4 (for example, see GenBank® Accession No. YP_009742611 or YP_009725300); Nsp6 (for example, see GenBank® Accession No. YP_009742613 or YP_009725302); Nsp7 (for example, see GenBank® Accession No. YP_009742614 or YP_009725303); Nsp8 (for example, see GenBank® Accession No. YP_009742615 or YP_009725304); Nsp9 (for example, see GenBank® Accession No. YP_009742616 or YP_009725305); Nsp10 (for example, see GenBank® Accession No. YP_009742617 or YP_009725306), Nsp11 (for example, see GenBank® Accession No. YP_009725312); 3C-like proteinase (for example, see GenBank® Accession No. YP_009742612 or YP_009725301); leader protein (for example, see GenBank® Accession No. YP_009742608 or YP_009725297); ORF7b (for example, see GenBank® Accession No. YP_009725318); 2′-O-ribose methyltransferase (for example, see GenBank® Accession No. YP_009725311); endoRNAse (for example, see GenBank® Accession No. YP_009725310); 3′-to-5′ exonuclease (for example, see GenBank® Accession No. YP_009725309); helicase (for example, see GenBank® Accession No. YP_009725308); RNA-dependent RNA polymerase (for example, see GenBank® Accession No. YP_009725307); orf1a polyprotein (for example, see GenBank® Accession No. YP_009725295); ORF10 protein (for example, see GenBank® Accession No. YP_009725255); ORF8 protein (for example, see GenBank® Accession No. YP_009724396); ORF7a protein (for example, see GenBank® Accession No. YP_009724395); ORF6 protein (for example, see GenBank® Accession No. YP_009724394); ORF3a (for example, see GenBank® Accession No. YP_009724391); and orflab polyprotein (for example, see GenBank® Accession No. YP_009724389). In some embodiments, the pepmixes are obtained commercially (for example, AP3A, NCAP, NS6, NS7A, NS7B, NS8, ORF10, ORF9B, Spike Glycoprotein, VEMP, VME1 and Y14 pepmixes from JPT Peptide Technologies (Berlin, Germany)).

In embodiments wherein the T cells are directed against MERS instead of SARS-CoV-2, the T cells may target any protein or proteins expressed from the MERS virus, and in specific cases the proteins include (as examples) one or more of the spike (S; for example, see GenBank® Accession No. QBM11748), nucleocapsid (N; for example, see GenBank® Accession No. QBM11755), membrane (M; for example, see GenBank® Accession No. QBM11754), envelope (E; for example, see GenBank® Accession No. QBM11753), or any proteins of ORF1ab, ORF1a, ORF3, ORF4a, ORF4b, ORF5, or ORF8b, for example.

In embodiments wherein the T cells are directed against SARS-CoV instead of SARS-CoV-2, the T cells may target any protein or proteins expressed from the SARS-CoV virus, and in specific cases the proteins include (as examples) one or more of the spike (S; for example, see GenBank® Accession No. AAZ67052), nucleocapsid (N; for example, see GenBank® Accession No. ABD75315), membrane (M; for example, see GenBank® Accession No. ABD75325), envelope (E; for example, see GenBank® Accession No. ABD75324), or any proteins of ORF1a, ORF1b, ORF6, 3a, 3b, 6, 7a, 7b, 8a, 8b, or 9b, for example.

### A. Starting Population of T Cells

The starting population of T cells may be isolated from one or more subjects, particularly mammalian subjects and including human subjects. The starting population of T cells can be isolated and expanded from one or more donor samples, such as an allogeneic sample, or from the subject who will receive the cells (i.e., autologous). The starting population of T cells can be obtained from a subject of interest, such as a subject who is healthy, a subject who lacks SARS-CoV-2 infection, a subject who has antibodies to SARS-CoV-, a subject who has tested positive for SARS-CoV-2, a subject who has tested negative for SARS-CoV-2, a subject who has a relative or comes into close contact with one or more individuals that have SARS-CoV-2 infection, one or more subjects that were SARS-CoV-2-positive followed by being SARS-CoV-2-negative, one or more subjects that have antibodies to SARS-CoV-2, one or more subjects that lack antibodies to SARS-CoV-2, a subject suspected of having a particular disease or condition, a subject suspected of having a predisposition to a particular disease or condition, a subject who is undergoing therapy for a particular disease or condition or a combination thereof. Any test for the presence or absence of SARS-CoV-2 may be utilized. In specific embodiments, the test is a molecular test (such as nucleic acid amplification test (NAAT), RT-PCR test, or LAMP test tests) that detect the virus’s genetic material, or antigen tests that detect specific proteins from the virus, such as on the surface of the virus. The starting population of T cells can be collected from any location in which they reside in the subject including, but not limited to, blood, cord blood, spleen, thymus, lymph nodes, bone marrow, hematopietic stem cells, induced pluripotent stem cells (iPSCs), or a combination thereof. The isolated starting population of T cells may be used directly following procurement, or they can be stored for a period of time, such as by freezing. Such frozen, stored cells may or may not be further modified following thawing.

The starting population of T cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures, as examples. Tissues/organs from which the immune cells are enriched, isolated, and/or purified may be isolated from both living and non-living subjects, wherein the non-living subjects are organ donors. In particular embodiments, the starting population of T cells are isolated from blood, such as peripheral blood or cord blood. In some aspects, starting populations of T cells isolated from cord blood have enhanced immunomodulation capacity, such as measured by CD4-positive or CD8-positive T cell suppression. In specific aspects, the starting population of T cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects). In other embodiments, the T cells are derived from hematopietic stem cells, such as induced pluripotent stem cells (iPSCs).

When the population of immune cells is obtained from a donor distinct from the subject, the donor is preferably allogeneic, provided the cells obtained are subject-compatible in that they can be introduced into the subject. Allogeneic donor cells may or may not be human-leukocyte-antigen (HLA)-compatible.

In some aspects, the cells are human cells. The cells typically are primary cells, such as those isolated directly from a subject and/or isolated from a subject and frozen cells. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (T_{reg}) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, and/or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for one or more markers expressed or expressed to a relatively higher degree on one or more naive, memory, and/or effector T cell subpopulations.

Generally, the starting cell populations are isolated from blood drawn from a subject, for example using apheresis (e.g., leukapheresis) or venous puncture. In one example, blood is obtained from a donor subject, such as an HLA-matched donor or the same subject who is to receive the antigen-specific T cells (recipient subject). In one example, an HLA-matched donor is one that matches at least ⅙, such as 2/6, 3/6, 4/6 or particularly ⅚ or 6/6, of the HLA loci (such as the A, B, and DR loci). In particular examples, the HLA-matched donor is a first degree relative. Monocytes can be isolated from blood obtained from the subject using methods known in the art. In one example, monocytes are obtained by elutriation of monocytes. In another example, monocytes are obtained from peripheral blood mononuclear cells (PBMCs) using a kit to deplete nonmonocytic cells (for example from Miltenyi Biotec, Auburn, Calif.) or by positive selection using anti-CD14 magnetic beads as recommended by the manufacturer (Miltenyi Biotec). In another example, PBMCs are prepared by centrifugation over a Ficoll-Paque (Pharmacia, Uppsala, Sweden) density gradient and the monocytes separated from lymphocytes by counterflow centrifugation (for example using the J6-MC elutriator system; Beckman Instruments, Palo Alto, Calif.) or centrifugation on a continuous Percoll (Pharmacia, Piscataway, N.J.) density gradient.

Similarly, lymphocytes can be isolated from blood obtained from the subject using methods known in the art. In one example, lymphocytes are collected by elutriation of the lymphocytes. B cells can also be depleted. In another example, PBMCs are prepared by centrifugation over a Ficoll-Paque density gradient and the lymphocytes separated from monocytes as described above.

In some examples, a monocyte/lymphocyte population (a leukocyte pack or peripheral blood leukocytes (PBL)) is isolated from a subject. PBLs can be obtained by incubation of citrated blood in a medium that lyses erythrocytes, and removal of the lysed cells, thereby generating a PBL population. In one example, blood is incubated in NH₄Cl buffer (0.15 M NH₄Cl, 10 mM NaHCO₃ [pH 7.4]) for 5 minutes at 4° C. (this can be repeated three times), followed by a wash in Ca²⁺--Mg²⁺-free phosphate-buffered saline (PBS-A) supplemented with 0.035% (wt/vol) EDTA and centrifugation to remove the lysed erythrocytes. However, this method is exemplary, and other methods known to those of skill in the art can also be utilized. The resultant monocyte, lymphocyte, or monocyte/lymphocyte product can be cryopreserved prior to use, using standard methods (for example using a combination of Pentastarch and DMSO). In some examples, cells are cryopreserved in aliquots of 5 to 200×10⁶ cells/vial, such as 6-10×10⁶ monocytes/vial, such as 50-200×10⁶ lymphocytes/vial, such as 10-50×10⁶ PBL/vial. To qualify for cryopreservation, the cell culture ideally contains predominately monocyte, lymphocyte, or monocyte/lymphocyte cells by flow cytometry. Sterility of the population need not be determined at this stage of the target antigen-specific T cells generation procedure; such a determination can occur after the final co-culture of cells. Methods for obtaining other APC populations, such as dendritic and B lymphoblastoid cells, are known in the art. For example, the Blood Dendritic Cell Isolation Kit II (Miltenyi Biotec Inc., Auburn, Calif.) can be used to obtain dendritic cells from blood according to the manufacturer’s instructions or by culture from blood cells using the method of Wong et al. (Cytotherapy, 4: 65-76, 2002, herein incorporated by reference). B lymphoblastoid cells can be cultured from peripheral blood, for example using the method of Tosato (Current Protocols in Immunology, Ed Coligan et al, Wiley, 2007, Chapter 7:Unit 7.22, herein incorporated by reference).

### B. Expansion of SARS-CoV-2-Specific T Cells

The methods of the present disclosure may comprise exposing the starting population of T cells, such as the buffy coat of isolated peripheral blood mononuclear cells (PBMCs), to a peptide library (i.e., mixture) and one or more cytokines for a period of time sufficient to expand SARS-CoV-2-specific T cells. The peptide library may comprise overlapping peptides spanning one or more antigens selected from the group consisting of AP3A, NCAP, NS6, NS7A, NS7B, NS8, ORF10, ORF9B, Spike Glycoprotein, VEMP, VME1 and Y14, (and these prior 12 listings refer to specific pepmixes from JPT Peptide Technologies), spike (S, and also referred to as Surface protein), membrane (M); envelope (E); nucleocapsid (N); Nsp2; Nsp3; Nsp4; Nsp6; Nsp7; Nsp8; Nsp9; Nsp10; Nsp11; 3C-like proteinase; leader protein; ORF7b; 2′-O-ribose methyltransferase; endoRNAse; 3′-to-5′ exonuclease; helicase; RNA-dependent RNA polymerase; orf1a polyprotein; ORF10 protein; ORF8 protein; ORF7a protein; ORF6 protein; ORF3a; and orflab polyprotein.

The peptide library used in the methods described herein are libraries (and may also be called pepmixes) of overlapping peptides that span all or a portion of one or more SARS-CoV-2 protein sequences. The peptide mixture may include 1, 2, 3, 4, 5, 6, 7, 8, 9, 10, or more epitopes. In specific embodiments, the peptides are at least 10 amino acids long (for example, 10-30 amino acids, 12-18 amino acids, or 15-25 amino acids long). In some examples, the peptide mixture includes overlapping 15 amino acid peptides (15mers) that are arranged such that portions of the fragments and certain sequence of amino acids from the parent sequence occur in more than one peptide fragment of the mixture. The peptides may overlap with one another by 1, 2, 3, 4, 5, 6, 7, 8, 9, 10, 11, 12, 13, 14, 15, 16, 17, 18, 19, 20, or more amino acids, depending on their length. The peptides may overlap with one another by 10-15, 10-14, 10-13, 10-12, 10-11, 11-15, 11-14, 11-13, 11-12, 12-15, 12-14, 12-13, 13-15, 13-14, or 14-15 amino acids (for example, overlap by 10, 11, 12, 13, 14, or 15 amino acids). In one specific example, the peptides in the peptide mixture overlap by 11 amino acids. The peptide library may comprise peptides of 8 to 30, such as 8, 9, 10, 11, 12, 13, 14, 15, 16, 17, 18, 19, 20, 21, 22, 23, 24, 25, 26, 27, 28, 29, or 30 contiguous amino acids of a full length antigen, although longer peptides may be used. With knowledge of a target antigen sequence, immunogenic peptide sequences predicted to bind to a major histocompatibility complex molecule can be determined using publicly available programs. For example, an HLA binding motif program on the Internet (Bioinformatics and Molecular Analysis Section-BIMAS) can be used to predict epitopes of SARS-CoV-2 proteins, using routine methods.

In specific cases, the peptide mixture comprises pools of peptides of 15 amino acids in length with 11 amino acid overlap. A pool of peptides may comprise 50-200 peptides for each immunodominant protein, such as at least 50, 60, 70, 80, or 90 peptides per immunodominant protein. Exemplary peptide libraries for use in the present methods are commercially available as PEPMIX™ (JPT).

In one specific embodiment, 5 ng to 10 µg of peptide or each peptide library are employed per ml of culture, such as 5, 50, 100, 150, 200, 250, 300, 350, 400, 450, 500, 1500, 2000, 3000, 4000, 5000, 6000, 7000 or 8000 ng/ml, particularly 5000 ng of each peptide pool is used to stimulate cells (e.g., about 1×10⁶ to about 75×10⁶ cells, particularly 5 µg of each peptide pool per 50×10⁶ cells).

The culture may comprise two or more cytokines such as IL-2, IL-4, IL-7, IL-12, IL-18, IL-15, and/or IL-21. In particular aspects, the culture comprises IL-7, IL-15, and IL-2. In some examples, the one or more cytokines are added to the culture medium at a concentration of about 2 ng/ml to about 100 ng/ml (for example, about 2 ng/ml to about 50 ng/ml, about 5 ng/ml to about 20 ng/ml, or about 10 ng/ml to about 20 ng/ml). In other examples, the one or more cytokines are added to the culture medium at a concentration of about 5-100 U/ml (such as about 10 U/ml to about 50 U/ml, about 20 U/ml to about 100 U/ml, or about 10 U/ml to about 20 U/ml). In particular aspects, IL-7 is present in the culture at a concentration of about 1 to 25 ng/mL, such as about 5, 10, or 15 ng/mL, particularly about 10 ng/mL. IL-2 may be present at a concentration of about 1 to 50 iU/mL, such as about 5, 10, 15, 20, 25, or 30 iU/mL, particularly about 20 iU/mL. Further, IL-15 may be present at a concentration of about 1 to 25 ng/mL, such as about 5, 10, or 15 ng/mL, particularly about 10 ng/mL. Additional cytokines may include, but are not limited to, IL-1, IL-4, and/or IL-6.

The length of time for expansion of the SARS-CoV-2-specific T cells may be any suitable length, such as about 7-21 days, including 7, 8, 9, 10, 11, 12, 13, or 14 days up to 21 days or longer. In particular aspects, the T cells are cultured in the presence of the peptide library and cytokines for about 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, or 13-14 days.

In one exemplary method, 50×10⁶ PBMCs are suspended at 1.times.10.sup.6 cells/mL in T cell expansion media (e.g., RPMI 1640 (45%), Clicks-EHAA media (45%), and human AB serum (10%), supplemented with GlutaMax at 2 mM). The cells are plated at a concentration of about 2×10⁶ cells/mL per well in a 24-well plate for the first 4 days of expansion, followed by transfer to a tissue culture flask (e.g., 75 cm², 100 cm₂, or 150 cm² flask) for the remainder of the expansion of the SARS-CoV-2-specific T cells.

In particular embodiments, the method for expanding SARS-CoV-2-specific T cells does not comprise the use of a second population of cells which have been previously exposed to, stimulated with, or primed with SARS-CoV-2 protein(s), such as the peptide library. For example, the second population of cells may comprise antigen presenting cells, dendritic cells, monocytes, PBMCs, or B cell lymphocytes that have been stimulated with one or more of the peptide libraries. Instead, embodiments of the present disclosure concern direct stimulation and expansion of SARS-CoV-2-specific T cells without the need for a pre-stimulation step.

The rapid expansion method may provide an increase in the number of SARS-CoV-2-specific T-cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-fold, or greater) over a period of about 10 to about 14 days.

T cell expansion may be evaluated by counting viable CD3⁺ cells (i.e. the target population of cells is CD3⁺, in at least specific cases). Viable cells can be tested by cell staining with Trypan blue (and light microscopy) or 7-amino-actinomycin D, vital dye emitting at 670 nm (or ViaProbe a commercial ready-to-use solution of 7AAD) and flow cytometry, employing a technique known to those skilled in the art. Where the stain penetrates into the cells, the cells are considered not viable. Cells which do not take up dye are considered viable.

Purity of the population of SARS-CoV-2-specific T cells can be determined using routine methods. In one example, purity is determined using markers present on the surface of SARS-CoV-2-specific T cells. Antigen-specific T cells are positive for the CD3 marker, along with the CD4 or CD8 marker, and IFN-gamma (which is specific for activated T cells). For example, fluorescence activated cell sorting (FACS) can be used to identify (and sort if desired) populations of cells that are positive for CD3, CD4/CD8, and IFN-gamma by using differently colored anti-CD3, anti-CD4, anti-CD8 and anti-IFN-gamma. Briefly, stimulated SARS-CoV-2-specific T cells are incubated in the presence of anti-CD3, anti-CD4, anti-CD8 and anti-IFN-.gamma. (each having a different fluorophore attached), for a time sufficient for the antibody to bind to the cells. After removing unbound antibody, cells are analyzed by FACS using routine methods.

The expanded SARS-CoV-2-specific T cells may be characterized to demonstrate specificity for SARS-CoV-2. The characterization may comprise determining the percentage of CD4⁺ and/or CD8+ T cells in the total population of CD3⁺ T cells. The population may comprise at least 80% CD4⁺ or CD8⁺ T cells, such as at least 81, 82, 83, 84, 85, 86, 87, 88, 89, 90, 91, 92, 93, 93, 95, 96, 97, 98, 99, or 100% CD4⁺ or CD8⁺ T cells. In particular aspects, the population of SARS-CoV-2-specific T cells produced by the present methods comprises at least 10% IFNgamma-producing cells, such as at least 11, 12, 13, 14, 15, 16, 17, 18, 19, 20, 21, 22, 23, 24, 25, or higher percentage of IFNgamma-producing cells. In some aspects, the population of SARS-CoV-2-specific T cells produced by the present methods comprises at least 5% IL-2-producing cells, such as at least 6, 7, 8, 9, 10, 11, 12, 13, 14, 15, or higher percentage of IL-2-producing cells. Thus, the population of SARS-CoV-2-specific T cells produced by the present methods may comprise at least 2% cells positive for both IFNgamma and IL2, particularly at least 3, 4, 5, 6, 7, 8, 9, 10, 11, 12, 13, 14, 15, or higher percentage of IFNgamma⁺IL2⁺ T cells.

The cytotoxicity of the SARS-CoV-2-specific T cells may also be determined. Methods for determining cytotoxicity are known in the art, for example a ⁵¹Cr-release assay (for example see Walker et al., 1987; Qin et al., 2002; both herein incorporated by reference).

### C. Genetically Engineered SARS-CoV-2-Specific T Cells

The SARS-CoV-2-specific T cells of the disclosure may be genetically engineered with one or more modifications, including to enhance activity of the cells. In some cases, the cells will be enhanced for activity to target the SARS-CoV-2, for efficacy at killing the virus, and/or for enhancing persistence of the cells under exposure to one or more hazardous conditions, such as being exposed to one or more drugs that would be cytolytic to the cells.

In one embodiment, the cells are engineered to express one or more antigen receptors that can target one or more viral antigens, such as engineered T cell receptors (TCRs) and/or chimeric antigen receptors (CARs). For example, the host cells (e.g, autologous or allogeneic T cells) may be modified to express a TCR or CAR having antigenic specificity for a SARS-CoV-2antigen. Multiple CARs and/or TCRs, such as to different SARS-CoV-2 antigens, may be added to the SARS-CoV-2-specific T cells.

Suitable methods of modification are known in the art. See, for instance, Sambrook and Ausubel, supra. For example, the cells may be transduced to express a T cell receptor (TCR) having antigenic specificity for a viral antigen using transduction techniques described in Heemskerk et al., 2008 and Johnson et al., 2009.

Electroporation of RNA coding for the full length TCR alpha and beta (or gamma and delta) chains can be used as alternative to overcome long-term problems with autoreactivity caused by pairing of retrovirally transduced and endogenous TCR chains. Even if such alternative pairing takes place in the transient transfection strategy, the possibly generated autoreactive T cells will lose this autoreactivity after some time, because the introduced TCR alpha and beta chain are only transiently expressed. When the introduced TCR alpha and beta chain expression is diminished, only normal autologous T cells are left. This is not the case when full length TCR chains are introduced by stable retroviral transduction, which will never lose the introduced TCR chains, causing a constantly present autoreactivity in the patient.

In some embodiments, the cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

In some embodiments, the CAR comprises an extracellular antigen-recognition domain that specifically binds to a SARS-CoV-2 antigen. In some embodiments, the antigen is a protein expressed on the surface of the virus. In some embodiments, the CAR is a TCR-like CAR and the antigen is a processed peptide antigen, such as a peptide antigen of an intracellular protein, which, like a TCR, is recognized on the cell surface in the context of a major histocompatibility complex molecule.

Exemplary antigen receptors, including CARs and recombinant TCRs, as well as methods for engineering and introducing the receptors into cells, include those described, for example, in International Patent Application Publication Numbers WO200014257, WO2013126726, WO2012/129514, WO2014031687, WO2013/166321, WO2013/071154, WO2013/123061 U.S. Pat. Application Publication No. US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European Patent Application Number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

### 1. Chimeric Antigen Receptors

In some embodiments, the chimeric antigen receptor comprises: a) an intracellular signaling domain, b) a transmembrane domain, and c) an extracellular domain comprising an antigen binding region.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding a SARS-CoV-2 antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the disclosure includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, and 4-1BB (CD137). In addition to a primary signal initiated by CD3zeta, an additional signal provided by a human costimulatory receptor inserted in a human CAR is important for full activation of NK cells and could help improve in vivo persistence and the therapeutic success of the adoptive immunotherapy.

In some embodiments, the CAR is constructed with a specificity for a particular SARS-CoV-2 antigen (or marker or ligand). Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a SARS-CoV-2-specific antigen binding domain.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into T cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into the T cells. Suitable vectors for use in accordance with the method of the present disclosure are non-replicating in the T cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

### 2. T Cell Receptor (TCR)

In some embodiments, the genetically engineered antigen receptors include recombinant TCRs and/or TCRs cloned from naturally occurring T cells. A “T cell receptor” or “TCR” refers to a molecule that contains a variable alpha and beta chains (also known as TCRalpha and TCRbeta, respectively) or a variable gamma and delta chains (also known as TCRgamma and TCRdelta, respectively) and that is capable of specifically binding to an antigen peptide bound to a MHC receptor. In some embodiments, the TCR is in the alpha/beta form.

Typically, TCRs that exist in alpha/beta and gamma/delta forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the alpha/beta form or gamma/delta form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable alpha chain and variable beta chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the .beta.-chain can contain a further hypervariability (HV4) region.

In some embodiments, the TCR chains contain a constant domain. For example, like immunoglobulins, the extracellular portion of TCR chains (e.g., a-chain, (3-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or Cₐ, typically amino acids 117 to 259 based on Kabat, beta-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the .alpha. and .beta. chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (gamma, delta, and epsilon) in mammals and the zeta-chain. For example, in mammals the complex can contain a CD3gamma chain, a CD3delta chain, two CD3epsilon chains, and a homodimer of CD3zeta chains. The CD3gamma, CD3delta, and CD3epsilon chains are highly related cell surface proteins of the immunoglobulin superfamily containing a single immunoglobulin domain. The transmembrane regions of the CD3gamma, CD3delta, and CD3epsilon chains are negatively charged, which is a characteristic that allows these chains to associate with the positively charged T cell receptor chains. The intracellular tails of the CD3gamma, CD3delta, and CD3epsilon chains each contain a single conserved motif known as an immunoreceptor tyrosine-based activation motif or ITAM, whereas each CD3zeta chain has three. Generally, ITAMs are involved in the signaling capacity of the TCR complex. These accessory molecules have negatively charged transmembrane regions and play a role in propagating the signal from the TCR into the cell. The CD3- and zeta-chains, together with the TCR, form what is known as the T cell receptor complex.

In some embodiments, the TCR may be a heterodimer of two chains alpha and beta (or optionally gamma and delta) or it may be a single chain TCR construct. In some embodiments, the TCR is a heterodimer containing two separate chains (alpha and beta chains or gamma and delta chains) that are linked, such as by a disulfide bond or disulfide bonds. In some embodiments, a TCR for a target antigen (e.g., a viral antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, a high-affinity T cell clone can be isolated from a patient, and the TCR isolated. In some embodiments, the T-cells can be a cultured T-cell hybridoma or clone. In some embodiments, the TCR clone for a target antigen has been generated in transgenic mice engineered with human immune system genes (e.g., the human leukocyte antigen system, or HLA). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

### 3. Suicide Genes

The SARS-CoV-2-specific T cells of the present disclosure may comprise one or more suicide genes. The term “suicide gene” as used herein is defined as a gene which may be used to selectively target cells for killing. For example, as suicide gene may, upon administration of a prodrug, effect transition of a gene product to a compound which kills its host cell. Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

The E. coli purine nucleoside phosphorylase, a so-called suicide gene which converts the prodrug 6-methylpurine deoxyriboside to toxic purine 6-methylpurine. Other examples of suicide genes used with prodrug therapy are the E. coli cytosine deaminase gene and the HSV thymidine kinase gene.

Exemplary suicide genes include membrane bound TNFalpha, CD20, CD52, EGFRv3, or inducible caspase 9. In one embodiment, a truncated version of EGFR variant III (EGFRv3) may be used as a suicide antigen which can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-.alpha., .gamma.-lyase (MET), and Thymidine phosphorylase (TP).

### 4. Modification of Gene Expression

In some embodiments, the SARS-CoV-2-specific T cells of the present disclosure may be modified to have altered expression of certain genes such as glucocorticoid receptor, TGFbeta receptor (e.g., TGFbeta-RII), and/or CISH. In a specific embodiment, any T cells of the disclosure are modified to have altered expression of one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, TDAG8, Cbl-b, B2M, HLA class I, and CD7. In one embodiment, the SARS-CoV-2-specific T cells may be modified to express a dominant negative TGFbeta. receptor II (TGFbeta.RIIDN) which can function as a cytokine sink to deplete endogenous TGFbeta.

In some embodiments, the altered gene expression is carried out by effecting a disruption in the gene, such as a knock-out, insertion, missense or frameshift mutation, such as biallelic frameshift mutation, deletion of all or part of the gene, e.g., one or more exon or portion therefore, and/or knock-in. For example, the altered gene expression can be effected by sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

Exemplary gRNA sequences for CRISPR-Cas mediated knockdown of NR3CS (glucocorticoid receptor) include Ex3 NR3C1 sG1 5-TGC TGT TGA GGA GCT GGA-3 (SEQ ID NO:2) and Ex3 NR3C1 sG2 5-AGC ACA CCA GGC AGA GTT-3 (SEQ ID NO:3). Exemplary gRNA sequences for TGF-beta receptor 2 include EX3 TGFBR2 sG1 5-CGG CTG AGG AGC GGA AGA-3 (SEQ ID NO:4) and EX3 TGFBR2 sG2 5-TGG-AGG-TGA-GCA-ATC-CCC-3 (SEQ ID NO:5). The T7 promoter, target sequence, and overlap sequence may have the sequence TTAATACGACTCACTATAGG (SEQ ID NO:6)+target sequence+gttttagagctagaaatagc (SEQ ID NO:7).

In particular embodiments, the SARS-CoV-2-specific T cells are modified to be able to tolerate exposure to one or more compounds that would otherwise be harmful to the T cells. As an example only, patients having COVID+ pneumonia often develop acute respiratory distress syndrome (ARDS) and are given high-dose steroids to reduce the inflammation in the lungs. Steroids would be extremely cytolytic to the SARS-CoV-2-specific T cells. In specific embodiments, one can inactivate the glucocorticoid receptor (GR) in the SARS-CoV-2-specific T cells, using RNA-guided endonucleases CRISPR (clustered regularly interspaced short palindromic repeats) and CRISPR-associated (Cas) 9 strategy that targets exon 2 of the GR gene, as one illustrative example. As one example, the technique involves the expansion from donor blood of SARS-CoV-2-specific T cells as described herein, followed by CRISPR knockout of a desired gene, such as the NR3C1 gene encoding the GR (e.g., exon 2 of the NR3C1 gene. In other embodiments, cells are modified to have a gene knocked out and then are modified for specificity to SARS-CoV-2.

### IV. Methods of Treatment or Prevention With SARS-CoV-2-Specific T Cells

The present disclosure provides methods of treatment or prevention of coronavirus infection, including SARS-CoV-2 infection, in an individual in need thereof. In specific aspects, methods of treatment and prevention for SARS-CoV-2 are provided for an individual in need thereof. The individual may or may not have acute respiratory distress syndrome or pneumonia.

Individuals in need of methods of treatment or prevention disclosed herein may be of any animal susceptible to coronavirus (e.g., SARS-CoV-2) infection, including mammals, and particularly humans. The individual may be of any race or gender or age. The individual may already have SARS-CoV-2infection or may be at risk for having SARS-CoV-2infection. An individual at risk for SARS-CoV-2 infection may be middle-aged or elderly (e.g., greater than about 50, 55, 60, 65, 70, 75, 80 85, 90, or 95 years of age); have an underling medical condition (heart disease, lung disease (including asthma, COPD, emphysema), are immune suppressed, HIV-positive, have kidney disease, have liver disease, are obese, have diabetes, etc.), live in a nursing home or long-term care facility, or a combination thereof. The individual may or may not have been tested for SARS-CoV-2 infection and upon testing the individual may or may not have tested positive. The individual may or may not be subject to methods and compositions of the disclosure as a part of routine health preventative measures. The individual may be subject to methods and compositions of the disclosure in advance of a condition and/or event, such as prior to exposure to groups of people, such as prior to or during travel (airplane, train, boat, etc.), prior to attendance of an entertainment event, prior to entering a school of any kind, as part of entering the military, as a requirement for a job, as a part of presence at a medical facility (such as hospitalization, nursing home facility, rehabilitation facility), and so forth. An individual negative for SARS-CoV-2infection may be subject to methods and compositions of the disclosure for the purpose of protecting another individual or other individuals.

In particular embodiments, an effective amount of one or more of the compositions encompassed herein are provided to an individual that has SARS-CoV-2 infection, that is suspected of having SARS-CoV-2infection, or that is at risk for having SARS-CoV-2 infection. In certain embodiments, the one or more cell therapy compositions of the disclosure inhibit viral replication, infectivity, and/or induces host innate immunity from SARS-CoV-2. In particular embodiments, methods are encompassed herein in which administration of the SARS-CoV-2-specific T cells elicits an immune response in an individual for SARS-CoV-2 infection or enhancing an immune response in an individual for SARS-CoV-2 infection.

Specific embodiments include methods of producing an immune response to SARS-CoV-2 in a subject, comprising administering to the subject an effective amount of SARS-CoV-2-specific T cells as an immunogenic composition or vaccine composition. In some embodiments, there are methods for inducing a sustained immune response in an individual having SARS-CoV-2 infection by delivering to the individual an effective amount of SARS-CoV-2-specific T cells.

In some embodiments, methods and compositions of the disclosure utilize an additional viral therapy or preventative, including one or more additional coronavirus therapies or preventatives. In such cases, the additional viral therapy or preventative may be provided to the individual prior to the SARS-CoV-2-specific T cells, at the same time as the SARS-CoV-2-specific T cells of the disclosure, and/or after the SARS-CoV-2-specific T cells of the disclosure. The additional viral therapy or preventative may or may not be in the same formulation as the SARS-CoV-2-specific T cells of the disclosure. When the SARS-CoV-2-specific T cells are provided to an individual at a different time as an additional therapy or preventative, any suitable duration of time between their administrations may be utilized, including from 1-60 minutes, from 1-24 hours, from 1-7 days, from 1-4 weeks, from 1-12 months, any subrange therein, and so forth. In specific embodiments, the additional therapy or preventative is selected from the group consisting of Azithromycin, AC-55541, Apicidin, AZ3451, AZ8838, Bafilomycin A1, CCT 365623, Daunorubicin, E-52862, Entacapone, GB110, H-89, Haloperidol, Indomethacin, JQ1, Loratadine, Merimepodib, Metformin, Midostaurin, Migalastat, Mycophenolic acid, PB28, PD-144418, Ponatinib, Ribavirin, RS-PPCC, Ruxolitinib, RVX-208, S-verapamil, Silmitasertib , TMCB, UCPH-101, Valproic Acid, XL413, ZINC1775962367, ZINC4326719, ZINC4511851, ZINC95559591, 4E2RCat, ABBV-744, Camostat, Captopril, CB5083, Chloramphenicol, Chloroquine (and/or Hydroxychloroquine), CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/M1N128), FK-506 (Tacrolimus), Ternatin 4 (DA3), Tigecycline, Tomivosertib (eFT-508), Verdinexor, WDB002, Zotatifin (eFT226), antivirals, anti-retrovirals, broad spectrum antibiotics, tociluzumab, other biologics, and a combination thereof.

### V. Combination Therapy With NK Cells

In particular embodiments, the SARS-CoV-2-specific T cells are utilized with one or more other cell immunotherapies. In specific cases, the SARS-CoV-2-specific T cells are given to an individual at the same time as, before, and/or after the individual is administered viral-specific cells, including SARS-CoV-2-specific NK cells. The SARS-CoV-2-specific NK cells may be produced by a particular method.

In specific cases, the SARS-CoV-2-specific NK cells are genetically modified by the hand of man to (a) one or more engineered antigen receptors that target one or more proteins from the SARS-CoV-2 virus; (b). one or more engineered antigen receptors that target a receptor used by SARS-CoV-2 to enter a host cell; and/or (c) one or more engineered antigen receptors that target a ligand on the surface of a SARS-CoV-2-infected cell. The engineered antigen receptor may be a chimeric antigen receptor (CAR), an engineered T cell receptor (TCR), or both, and they may target a protein from the SARS-CoV-2 virus, such as the spike protein, membrane protein, envelope protein, nucleocapsid protein, Nsp2, Nsp3, Nsp4, Nsp6, Nsp7, Nsp8, Nsp9, Nsp10, Nsp11, 3C-like proteinase, leader protein, ORF7b, 2′-O-ribose methyltransferase, endoRNAse, 3′-to-5′ exonuclease, helicase, RNA-dependent RNA polymerase, orf1a polyprotein, ORF10 protein, ORF8 protein, ORF7 a protein, ORF6 protein, ORF3a, or orflab polyprotein. In some embodiments, the engineered antigen receptor targets angiotensin-converting enzyme-2 (ACE2) on a host cell or targets a ligand of Natural killer group 2D receptor (NKG2DR), such as MHC class I chain-related protein (MIC)A, MICB, UL16 binding protein 1 (ULBP), or Poliovirus Receptor (PVR).

In some embodiments, the SARS-CoV-2-specific NK cells encompassed by the disclosure are expanded by a particular method. In one example, the NK cells are expanded in an ex vivo method in which a starting population of mononuclear cells (MNCs) from cord blood is stimulated in the presence of antigen presenting cells (APCs) and IL-2, and in some cases there is re-stimulation of cells with APCs. In specific embodimens, the method is performed in a bioreactor, such as one having a gas-permeable membrane. In some cases, any cells are depleted for cells positive for CD3. In specific cases, there is no removal or addition of media components in the initial stimulation step. The APCs may or may not be gamma-irradiated, and they may or may not be engineered to express membrane-bound IL-21, and/or one or more of IL-15, IL-7, IL-18, and IL-2. In specific embodiments, the NK cells are expanded by stimulation with IL-2 and/or IL-7, IL-12, IL-15, IL-18, IL-21, or others.

In particular embodiments, the SARS-CoV-2-specific NK cells are produced following stimulation of cord blood MNCs in the presence of APCs that are universal APCs. In specific cases, the APCs are engineered to express CD48, CS1 (CD319) membrane-bound interleukin-21 (mbIL-21), and/or 41BB ligand (41BBL). In specific cases, the universal APCs have essentially no expression of endogenous HLA class I, II, or CD1d molecules.

In some cases, the SARS-CoV-2-specific NK cells have been expanded in the presence of an effective amount of universal antigen presenting cells (UAPCs), including in any suitable ratio. The NK cells may be cultured with the UAPCs at a ratio of 10:1 to 1:10; 9:1 to 1:9; 8:1 to 1:8; 7:1 to 1:7; 6:1 to 1:6; 5:1 to 1:5; 4:1 to 1:4; 3:1 to 1:3; 2:1 to 1:2; or 1:1, including at a ratio of 1:2, for example. In some cases, the NK cells were expanded in the presence of IL-2, such as at a concentration of 10-500, 10-400, 10-300, 10-200, 10-100, 10-50, 100-500, 100-400, 100-300, 100-200, 200-500, 200-400, 200-300, 300-500, 300-400, or 400-500 U/mL.

### EXAMPLES

The following examples are included to demonstrate particular embodiments of the disclosure. It should be appreciated by those of skill in the art that the techniques disclosed in the examples that follow represent techniques discovered by the inventor(s) to function well in the practice of the methods and compositions of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the disclosure.

### Example 1

### Rapid Generation of Off-The-Shelf, Third Party Covid19-Specific T Cells For Sars-Cov-2-Infected Patients

In one specific embodiment, off-the-shelf, third party SARS-CoV-2-specific T cells are produced by methods encompassed herein. In a specific example, a rapid VST expansion protocol is utilized to produce SARS-CoV-2-specific T cells. In one specific case, a culturing/expansion protocol utilizing about 10-14 days of culture generates SARS-CoV-2-specific T cells.

In one embodiment, peripheral blood mononuclear cells (such as from healthy controls) are cultured with different peptide libraries (including, e.g., 12 different peptide libraries) in which case 15 mers overlapping by 11 amino acids are utilized. The peptide libraries may span the entire sequence, or partial sequence, of one or more SARS-CoV-2 antigens, including AP3A, NCAP, NS6, NS7A, NS7B, NS8, ORF10, ORF9B, Spike Glycoprotein, VEMP, VME1 and Y14 that comprise the structural proteins spike (S), membrane (M), envelope (E) and nucleocapsid (N), as well as several proteins that are uncharacterized (JPT Peptide Technologies, Berlin, Germany) using GMP-compliant manufacturing methodologies. Effective immunogenic antigens and a useful cytokine cocktail are utilized (such as by comparing different combinations of IL-2, IL-4, IL-7, IL-12, IL-18, IL-15, and/or IL-21) for 10-14 days (as one example of duration of culturing) that yield the highest number of Th1-polarized, polyfunctional SARS-CoV-2-specific T-cells, including that are capable of selectively killing viral antigen-expressing target cells with no activity against non-infected autologous or allogeneic targets. At the end of culture, the cells are harvested and may be utilized directly or cryopreserved until use.

In one embodiment, one can rapidly generate a robust SARS-CoV-2 viral-specific therapy (VST) bank and characterize third-party, off-the shelf, most closely HLA-matched COVID-19+ CTLs in patients with intermediate or high-risk COVID-19 infections. In specific cases, patients receive 2 × 10⁵ CD3+ COVID-19+ T cells/Kg as a single infusion. Repeated VST infusions may be utilized to achieve complete remission, in some cases.

### Example 2

### An Example of Production of Sars-Cov-2-Specific T Cells

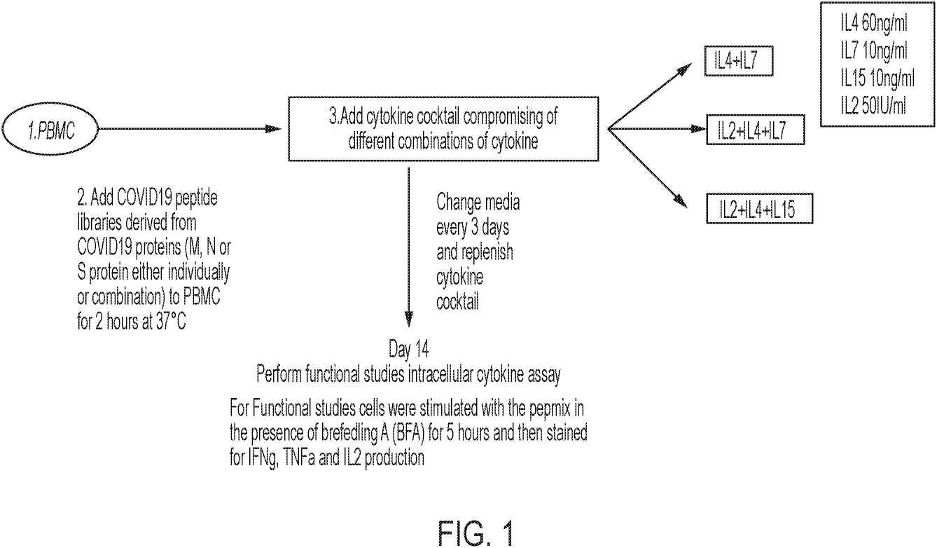

FIG. 1 illustrates one example of a production scheme for producing SARS-CoV-2-specific T cells. A starting population of PMBCs are directly stimulated with pepmixes spanning one or more SARS-CoV-2 (COVID-19) proteins. In this specific case, the proteins are M, N, S, or a combination thereof. At a particular time, a variety of cytokine cocktails are provided to the culture, and the media is changed and the cytokines are replenished at certain time points. On day 14, one or more particular functional studies are performed on the cells. As one example, the cells may be stained for IFNg, TNFa, and/or IL-2 production.

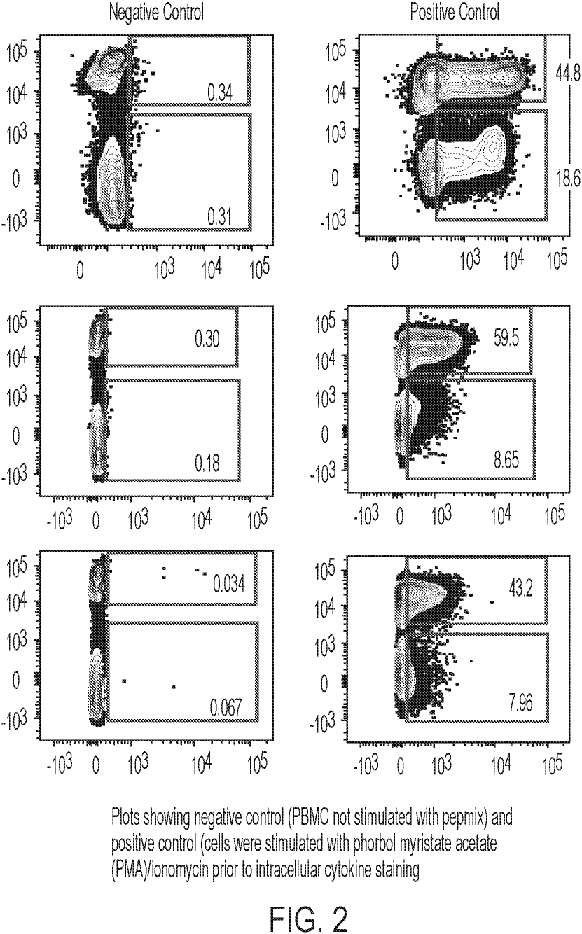

FIG. 2 demonstrates the ‘backgound’ intracellular interferon-gamma staining by T-cells in the absence of stimulation with pepmix (negative control- left panel) and in response to stimulation with PMA and inomycin (positive control).

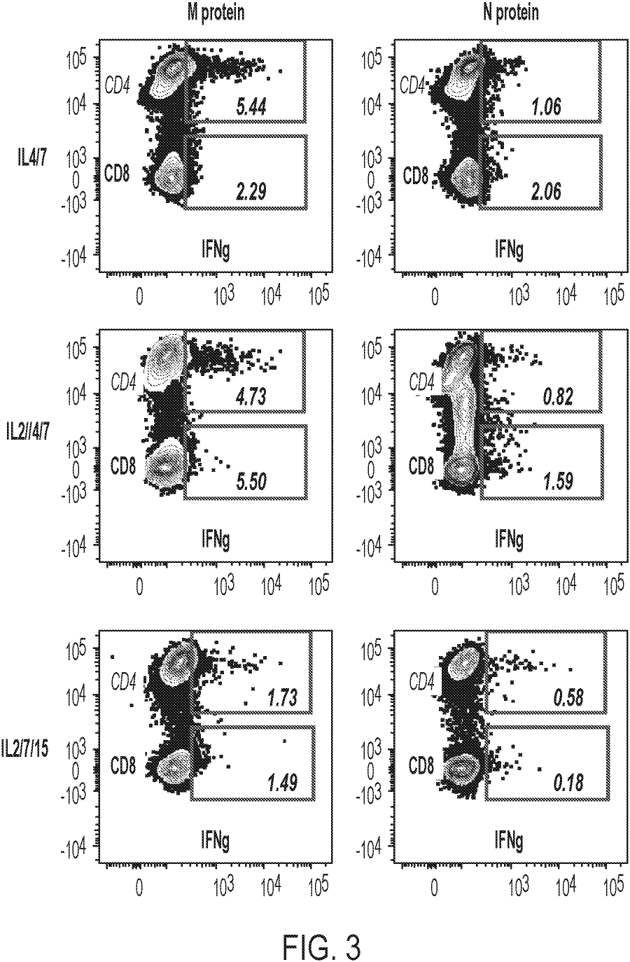

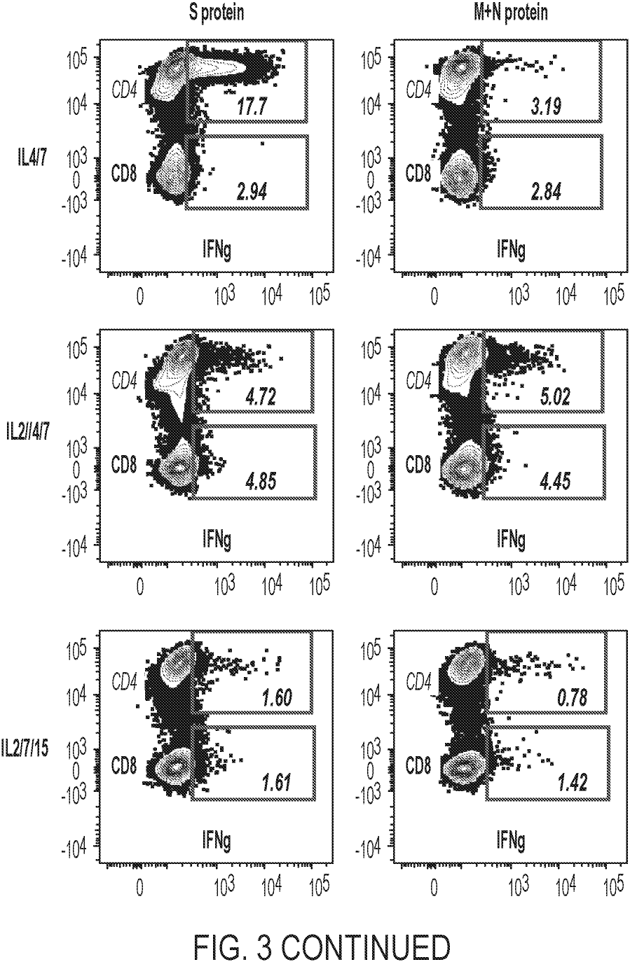

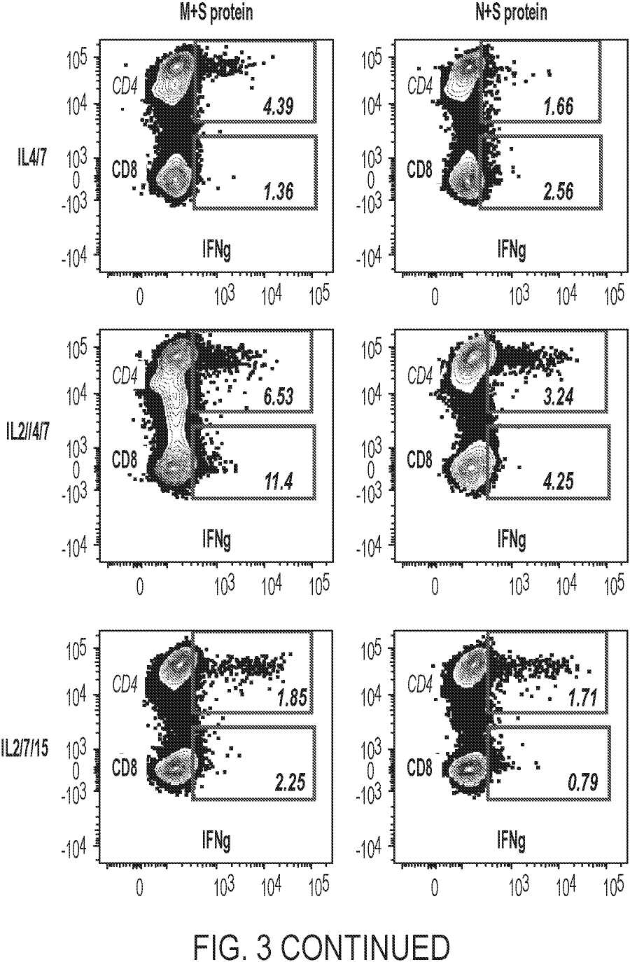

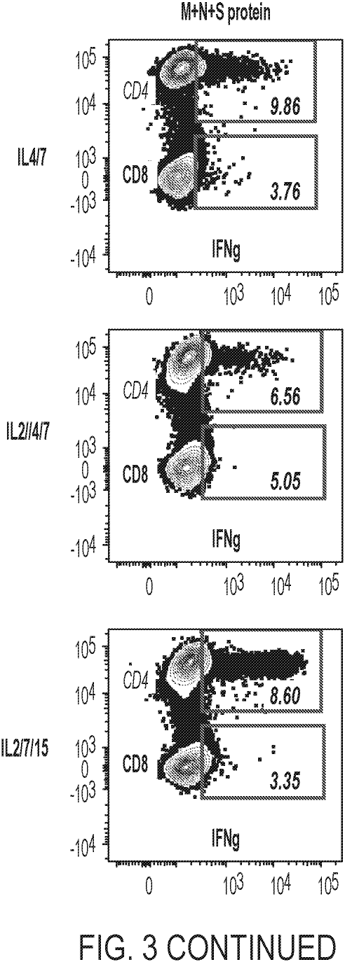

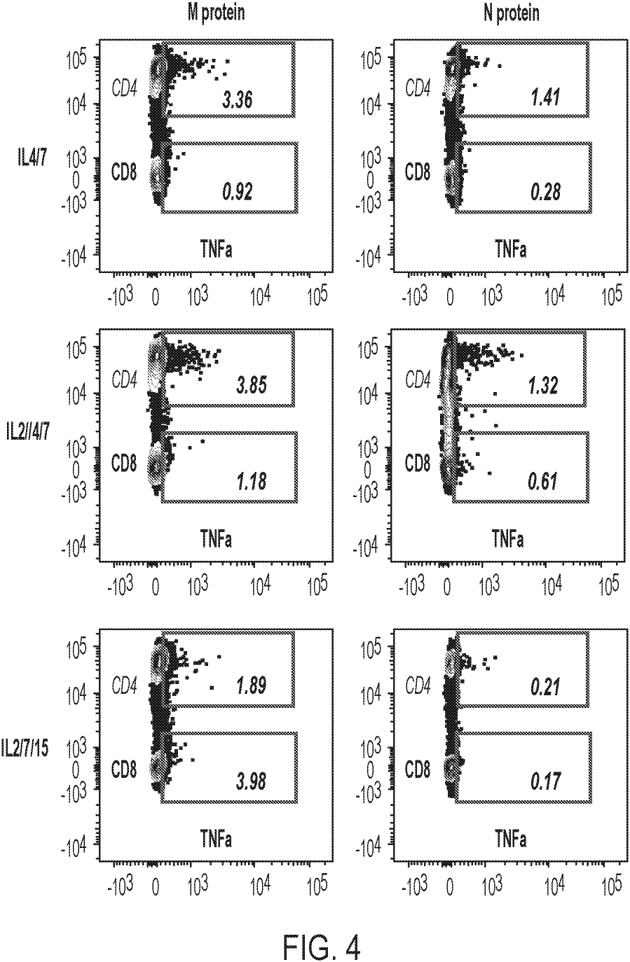

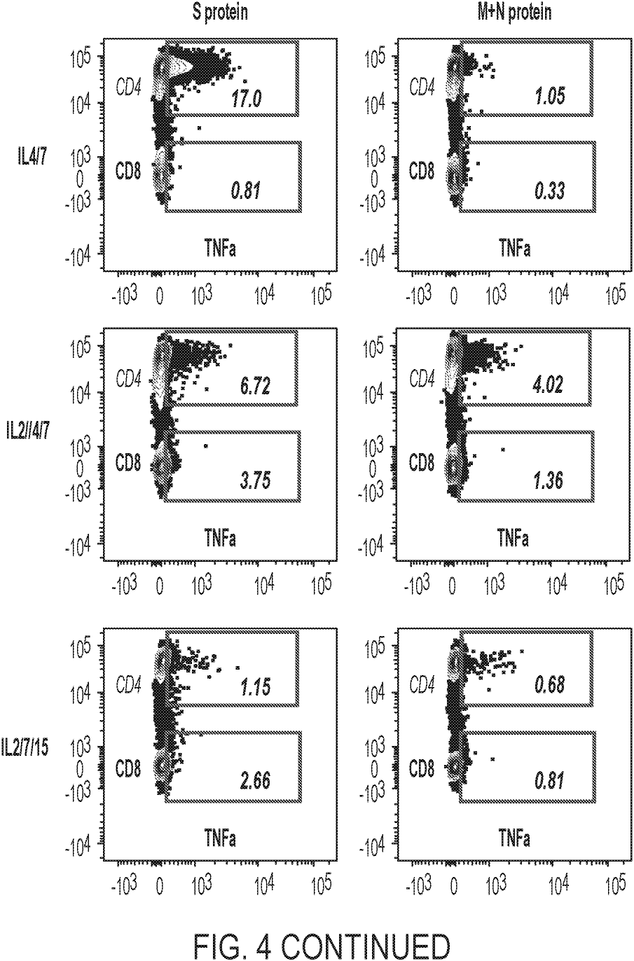

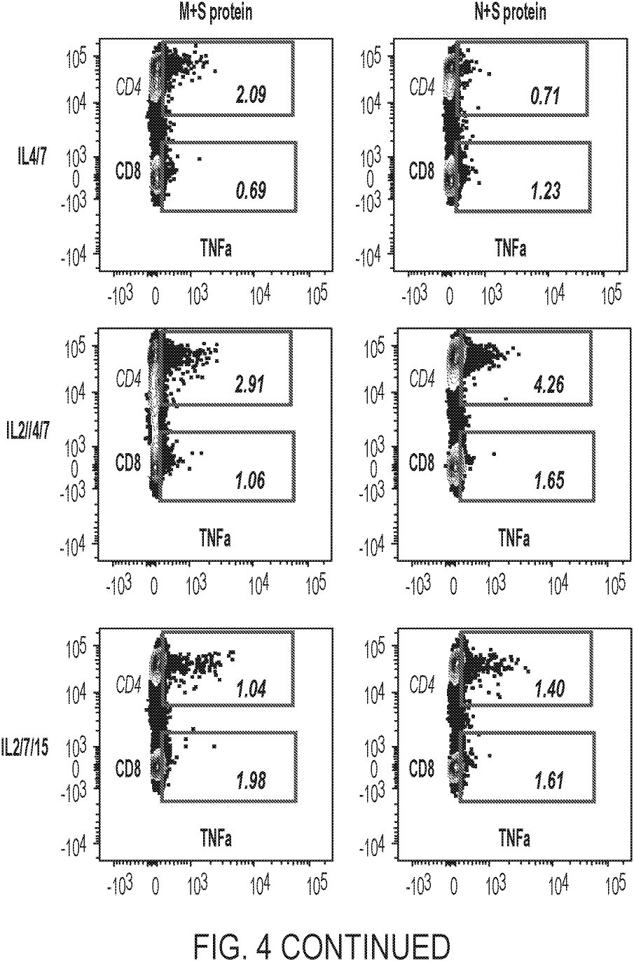

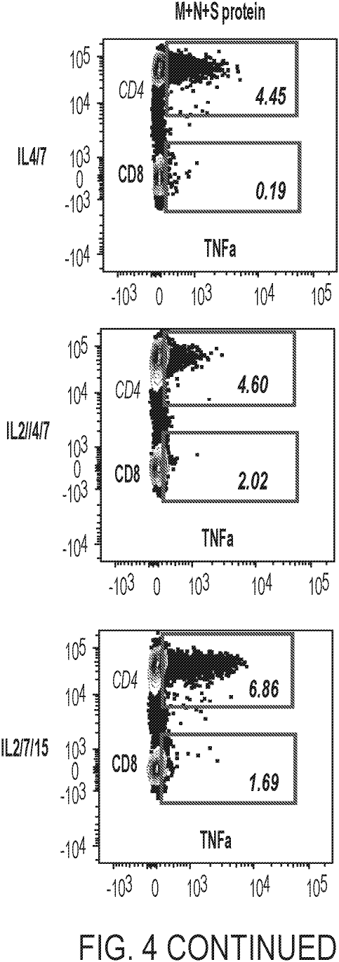

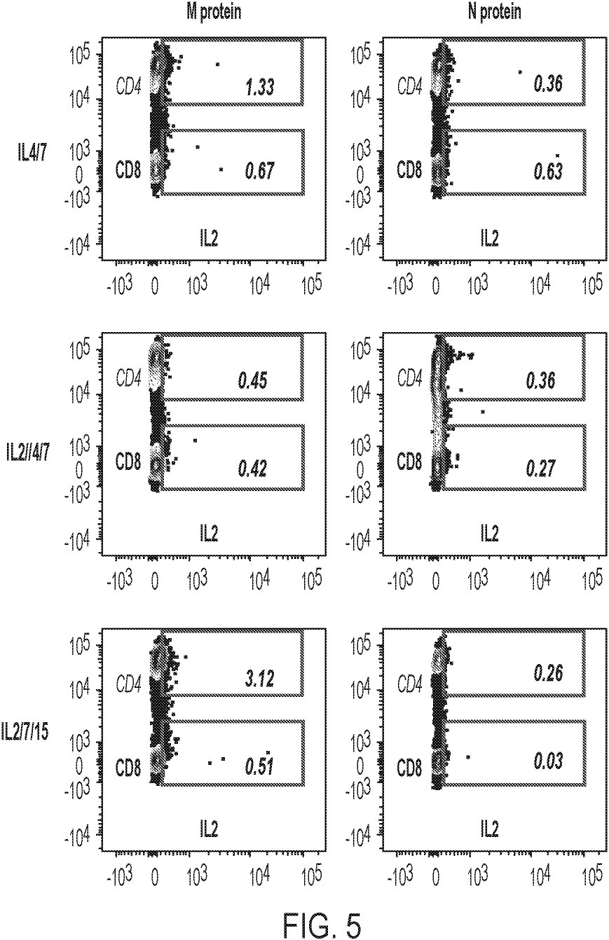

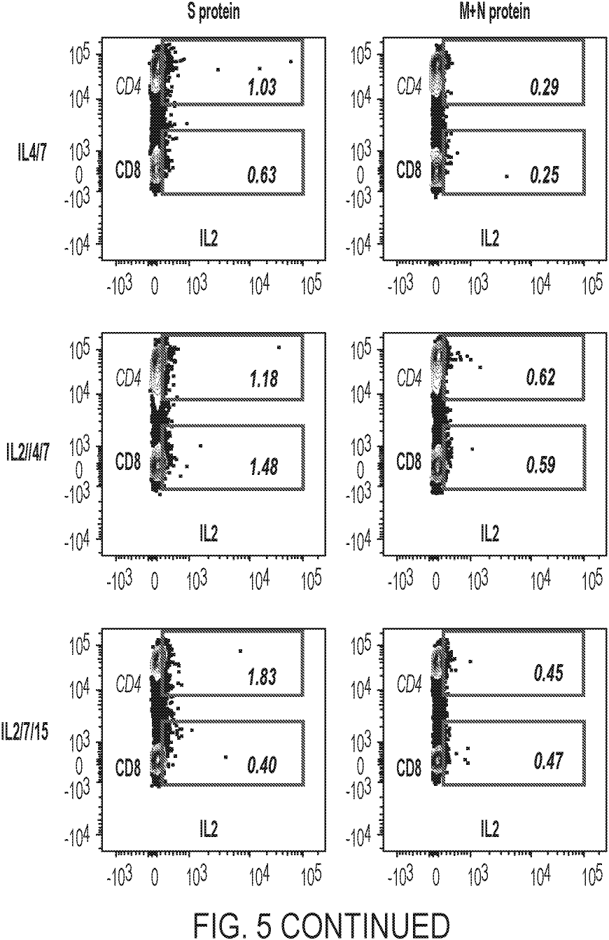

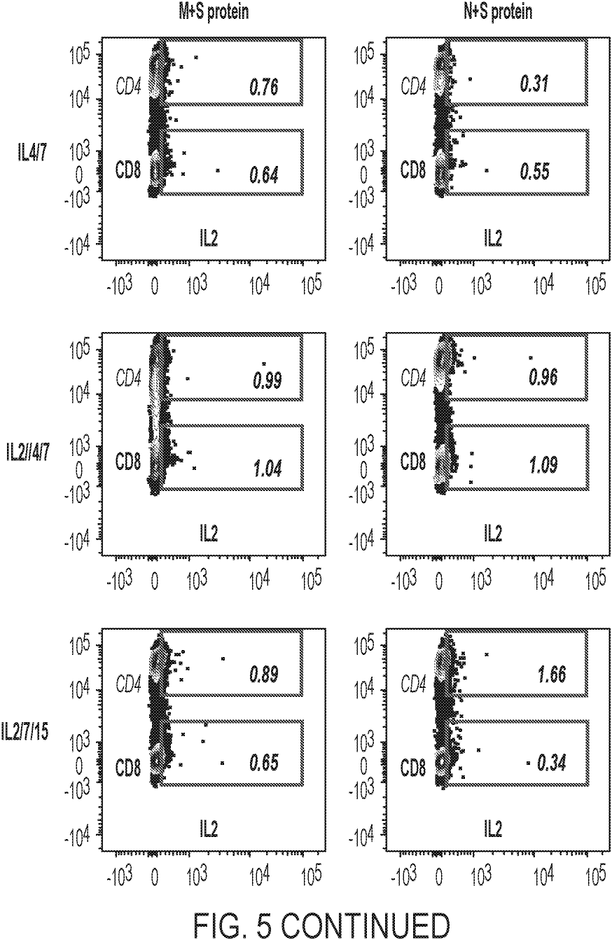

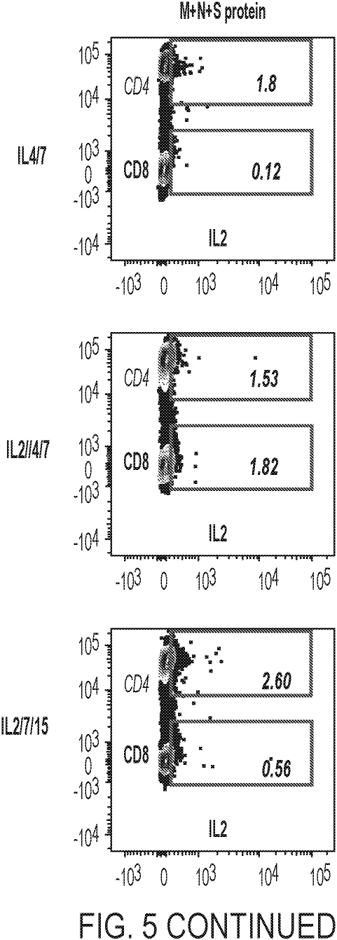

FIGS. 3-6 provide functional studies of the cells produced by the procedure described in FIG. 1 by ascertaining production of certain intracellular cytokines. For example, FIG. 3 demonstrates for a particular donor (Donor 1) the interferon (IFN) gamma response to ex vivo stimulation with COVID19 pepmixes. Briefly, PBMCs from Donor 1 were stimulated with pepmixes derived from the M, N and S proteins of COVID19, either individually or in combination, and cultured in the different noted cytokine cocktails. After 14 days of culture, over 17% of CD4+ T cells are directed against S protein and over 5% against M protein (FIG. 3). The TNF-alpha response for Donor 1 is shown in FIG. 4. As shown therein, 17% of CD4+ T cells are directed against the S protein and nearly 4% against the M protein. The IL-2 response for Donor 1 is provided in FIG. 5, where nearly 2% of CD4+ T cells are directed against the S protein and over 3% against the M protein.

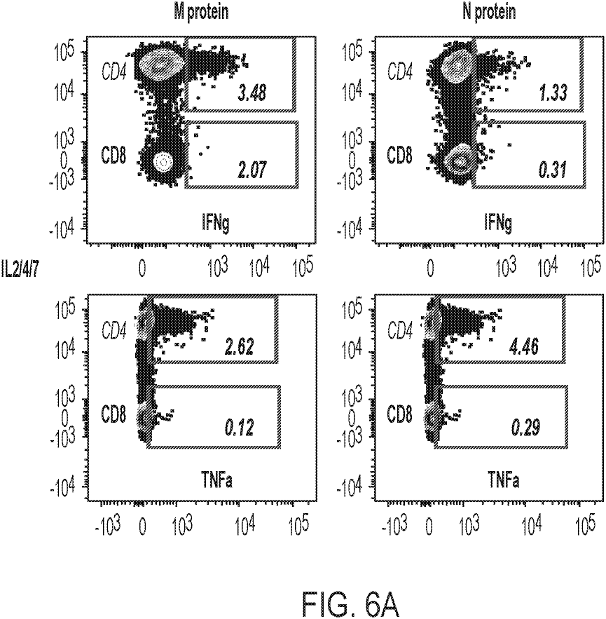

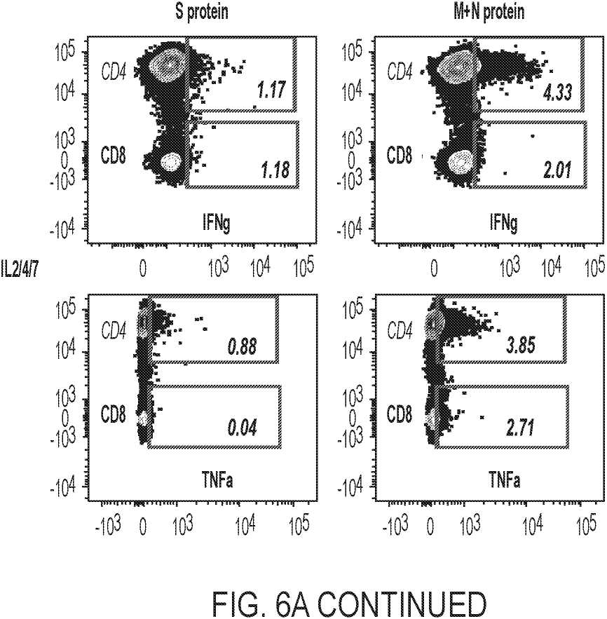

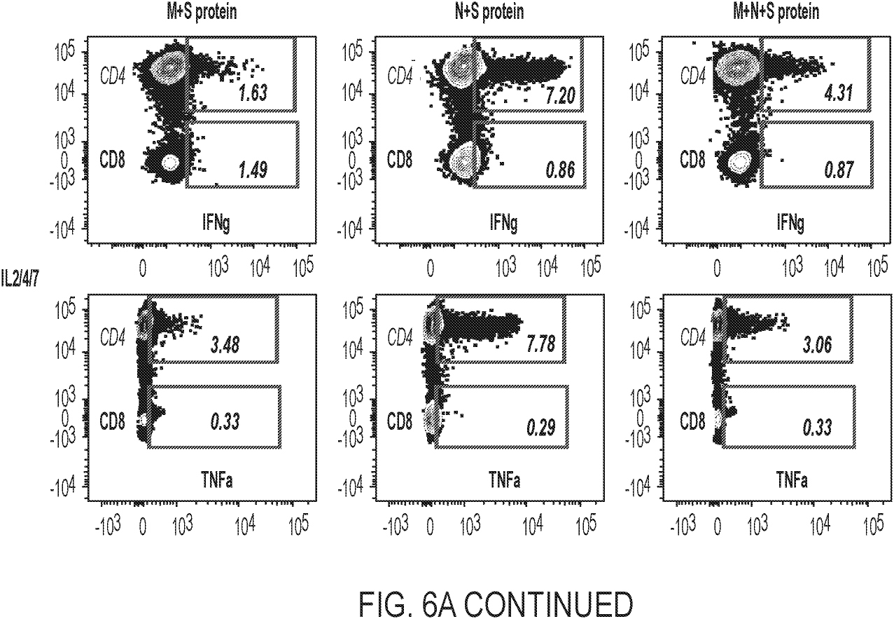

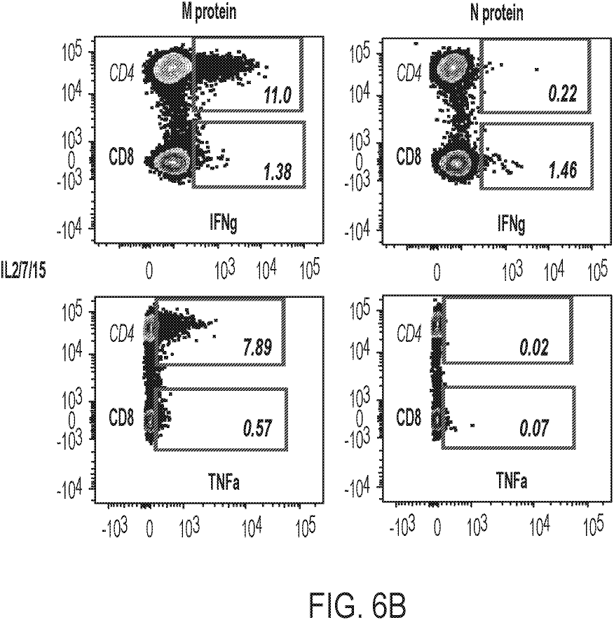

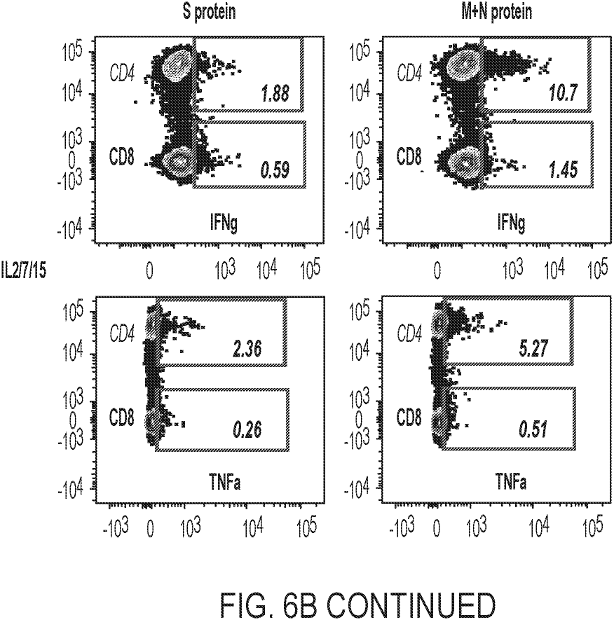

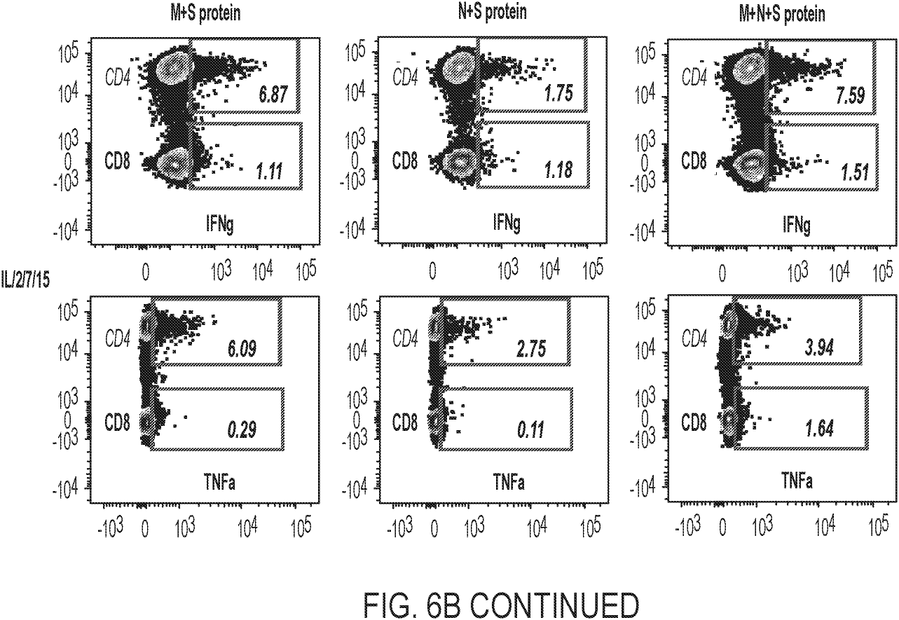

FIGS. 6A and 6B show the data for another donor (Donor 2). The IFN gamma and TNF-alpha responses are shown to ex vivo stimulation with COVID19 pepmixes. PBMCs were stimulated with pepmixes derived from the M, N and S proteins of COVID19, either individually or in combination, and cultured in different cytokine cocktails. After 14 days of culture, nearly 8% of CD4+ T cells are directed against the M protein and nearly 5% against the N protein. FIG. 6A shows expansion of COVID19 specific T cells in the presence of IL-2, IL-4, and IL-7. FIG. 6B shows expansion of COVID19 specific T cells in the presence of IL-2, IL-7, and IL-15.

### Example 3

### Generation of Glucocorticoid Resistant Sars-Cov-2 T-Cells For Adoptive Cell Therapy

### Overview

Adoptive cell therapy (ACT) with viral-specific T cells has been successfully used to treat a number of life-threatening viral infections, supporting the application of this approach to treat COVID-19. Thus, the inventors expanded SARS-CoV-2 T-cells from the peripheral blood of convalescent COVID-19 patients and non-exposed healthy donors (HDs) using different culture conditions and observed that the choice of cytokines modulates the expansion, phenotype and hierarchy of antigenic recognition by SARS-CoV-2 T-cells. Culture in the presence of IL-2/4/7 but not other cytokine-driven conditions resulted in >1000 fold expansion in SARS-CoV-2 T-cells with a retained hierarchy of response when compared to baseline (pre-expansion) samples. Single cell analysis confirmed that SARS-CoV-2 T cells are polyfunctional, with an activated Th1 phenotype and no evidence of exhaustion. The expanded CTLs were directed against the structural proteins of SARS-CoV-2, including the receptor-binding domain of the spike protein. Of note, SARS-CoV-2 T-cells could not be efficiently expanded from the peripheral blood of non-exposed HDs. Since corticosteroids are used for the management of severe COVID-19 and in order to facilitate clinical translation, the inventors developed an efficient strategy to inactivate the glucocorticoid receptor gene (NR3Cl) in SARS-CoV-2 CTLs using CRISPR-Cas9 gene editing.

### Methods

### COVID-19 Recovered Donors and Healthy Donors

Buffy coat units from 10 COVID-19 recovered donors (CoV-RD) and 20 mL of peripheral blood from 5 healthy donors were collected under local Institutional Review Board approved protocols (Lab02-0630 and PA13-0647) and following informed consent. All donors were 18 years or older and were recruited without consideration of disease severity, race, ethnicity or gender. All CoV-RD had recovered from proven symptomatic COVID-19 confirmed by a positive test for SARS-CoV-2. At the time of blood collection, all were asymptomatic with a negative PCR test, confirming full recovery.

Blood from Cov-RD was collected in heparin-coated blood bags and stored at room temperature prior to processing for peripheral blood mononuclear cell (PBMC) isolation. PBMCs were isolated by density-gradient sedimentation using Ficoll-Paque (Lymphoprep, Oslo, Norway). Isolated PBMCs were either used fresh for ex vivo expansion of SARS-CoV-2 specific T cells (SARS-CoV-2 CTLs) or cryopreserved in freeze media containing 10% DMSO (GIBCO), supplemented with 10% heat inactivated Human Serum AB (vendor information) and stored in liquid nitrogen until used for phenotypic and functional assays.

### Functional Assessment of SARS-CoV-2 Reactive T Cells

For intracellular assessment of cytokine production, cells were stimulated ex vivo with 15mer pepMixes overlapping by 11 amino acids derived from SARS-CoV-2 spike (S) (peptide pool 1 or 2), membrane (M), nucleocapsid (N), envelope (E), or the non-structural proteins (AP3A, Y14, NS6, NS7a, NS7B, NS8,ORF9B and ORF10) (JPT, Germany) [1 µg /ml per peptide] for 4 hours. Stimulation with an equimolar amount of DMSO was performed as negative control and with PMA-Ionomycin (1.25 ng/µl and 0.05 ng/µl, respectively) as positive control. Brefeldin A (BD Biosciences, San Diego, CA) was added into the culture for 4 hours. Cells were stained with an antibody cocktail containing CD3 APC Cy7 (Biolegend, Clone HIT3A), CD4 APC (E Biosciences, Clone SK3), CD8 PerCP Cy5.5 (Biolegend, Clone SK1), CD95 BV785 (Biolegend, Clone DX2) CD45RO BV650 (BD Biosciences, Clone UCHL1), CD45RA PECy7 (Biolegend, Clone HI100), CD27 BV711 (Biolegend, Clone O323), CCR7 FITC (BD Biosciences, Clone 150503) and CD62L BV605 (Biolegend, Clone DREG56) for 30 minutes on ice, then fixed and permeabilized using the BD fixation/permeabilization kit (BD Biosciences, San Diego, CA) according to manufacturer’s protocol. Cells were subsequently stained with antibodies against IL-2 PE (BD Biosciences, Clone MQ1-17H12), IFNγBV450 (BD Biosciences, Clone B27), and TNFα AF700 (Biolegend, Clone MAB11) for 30 mins. Following a final wash, cells were re-suspended in FACS buffer and data were acquired on a BD LSRFortessa (BD Biosciences). Data analysis was performed using Flowjo (Tree Star, Ashland, OR). The gates applied for the identification of IFNγ, IL-2, and TNFα on the total population of CD4+ and CD8+ T-cells were defined according to the negative control for each individual. Similar functional assays were performed for NR3C1 knockout (KO) CTLs.

### SARS-CoV-2 Antibody Assay

IgM and IgG responses against nucleocapsid, S1 receptor-binding domain (RBD), S1S2, S2, S1, OC43, HKU1, NL63 Nucleoprotein, and 229E Spike derived from SARS-CoV-2 and other human coronaviruses were performed at Genalyte (Houston, TX) CLIA-certified laboratory using plasma from convalescent patients.

### Cytokine and Chemokine Measurement

Cells were stimulated ex vivo with 15 mer pepMixes from S, M and N for 24 hours at 37° C. and 5% of CO2. Supernatants were collected and assayed with the Milliplex® MAP Human Cytokine/Chemokine panel (EMD Millipore Corporation, Burlington, MA) following the manufacturer’s instructions.

### Generation of SARS-CoV-2 Specific T-Cells

Isolated PBMC from CoV-RD and HD were pulsed with a SARS-CoV-2 pepMix (JPT, Germany) comprising the entire length of the structural (S, M, N, E) and non-structural (AP3A, Y14, NS6, NS7a, NS7B, NS8, ORF9B and ORF10) proteins at a concentration of 1 µg/ml per peptide. Cells were cultured in complete media with 5% human AB serum and supplemented with four different cytokine cocktails: IL-2 (50 IU/ml), IL-4 (60 ng/ml) and IL-7 (10 ng/ml) vs. IL-2 (50 IU/ml), IL-7 (10 ng/ml) and IL-15 (10 ng/ml) vs. IL-2 (50 IU/ml), IL-4 (60 ng/ml) and IL-21 (30 ng/ml) vs. IL-2 (50 IU/ml), IL-7 (10 ng/ml) and IL-21 (30 ng/ml) every 3 days. After 14 days of expansion, the frequencies of SARS-CoV-2 specific T-cells were determined by intracellular cytokine staining.

### Mass Cytometry

A panel of 40 metal-tagged antibodies was used for the in-depth characterization of SARS-CoV-2 reactive T-cells (Table 3). All unlabeled antibodies were purchased in carrier-free form (Fluidigm) and conjugated in-house with the corresponding metal tag using Maxpar X8 polymer per the manufacturer’s instructions (Fluidigm) and as previously described(Muftuoglu et al., 2018). Briefly, thawed PBMCs were rested overnight at 37° C. / 5% CO2 and stained with a freshly prepared antibody mix against cell surface markers for 30 minutes at room temperature on a shaker (100 rpm). For the last 3 minutes of incubation, cells were incubated with 2.5 µM cisplatin (Pt198, Fluidigm) for viability assessment, washed twice with cell staining buffer and fixed/permeabilized using BD Cytofix/Cytoperm solution for 30 minutes in dark at 4° C. Cells were washed twice with perm/wash buffer, stained with antibodies directed against intracellular markers and after an additional wash step, stored overnight in 500 µl of 1.6% paraformaldehyde (EMD Biosciences)/PBS with 125 nM iridium nucleic acid intercalator (Fluidigm). Samples were supplemented with EQ calibration beads (Fluidigm) and acquired at 300 events/second on a Helios instrument (Fluidigm) using the Helios 6.5.358 acquisition software (Fluidigm).

Mass cytometry data were normalized based on EQ™ four element signal shift over time using Fluidigm normalization software 2. Initial data processing was performed using Flowjo version 10.2. Calibration beads were gated out and singlets were chosen based on iridium 193 staining and event length. Dead cells were excluded by the Pt198 channel and manual gating was performed to select the CD45+CD3+ population which was subsequently exported for downstream analyses. A total of 156,384 cells were evenly sampled from 16 samples derived from 8 patients to perform automated clustering analysis. The data were processed using the R package cytofkit (v1.11.3). Expression values for each marker were arcsine transformed with a cofactor of 5. Data dimensionality reduction was performed using the R package Rtsne (v0.15) for t-Distributed Neighbor Embedding (tSNE) analysis. The R package Rphenograph (v0.99.1) was used to cluster all cells into 32 clusters. Both the R package Rstne (v0.15) and the R package Rphenograph (v0.99.1) were implemented in the R package cytofkit (v1.11.3). The t-SNE plots were generated using the R package ggplot2 (v3.3.2). Normalized mean values of marker expressions in each cluster were plotted as heatmap using the function “pheatmap” from R package pheatmap (v1.0.12). Min-max normalization was used to scale each marker’s mean expressions range to [0,1]. The normalized mean values of marker expressions were plotted as box plots using the function “ggpaired” from R package ggpubr (v0.4.0). The mean comparison p-values of Wilcoxon signed-rank test were added to the plots using the function “stat_compare_means” from R package ggpubr (v0.4.0).

### CRISPR-Cas9 Gene Editing of the Glucocorticoid Receptor

Knockout (KO) of NR3C1 (the glucocorticoid receptor gene) was performed on day 7 of T cell expansion using ribonucleoprotein (RNP) complex. The inventors used two crRNAs targeting exon 2 of the human NR3C1 gene: crRNA #1 TGAGAAGCGACAGCCAGTGA (SEQ ID NO:8), crRNA#2 GGCCAGACTGGCACCAACGG (SEQ ID NO:9) as previously described.(Basar et al., 2020) Briefly, Cas9 protein (IDT) and gRNA (crRNA + tracrRNA combination) were complexed and electroporated into 1 million SARS-CoV-2 specific T cells using the Neon transfection system (Thermo Fisher Scientific).

### Annexin V Apoptosis Assay

Annexin V apoptosis assay was performed to evaluate the effect of dexamethasone on the viability of CTLs from Cas9 control and NR3C1 KO groups. CTLs from both groups were treated with 200 µM dexamethasone (Sigma) for 72 hours. Cells were then collected, washed with annexin V buffer, and stained with annexin V (V500; BD Biosciences) and live/dead viability dye (efluor 660; Invitrogen) in addition to CD3 APC Cy7 (Biolegend, Clone HIT3A), CD4 APC (E Biosciences, Clone SK3), and CD8 PerCP Cy5.5 (Biolegend, Clone SK1). The proportion of apoptotic (positive for annexin V) and dead CTLs (positive for live/dead stain) was determined by flow cytometry.

### PCR Gel Electrophoresis

DNA was extracted and purified (QIAamp DNA Blood Mini Kit; Qiagen Inc) from SARS-CoV-2 specific T cells (control and NR3C1 KO conditions). We used the Platinum SuperFi Green PCR Master Mix from Invitrogen for polymerase chain reaction (PCR) amplification using the following PCR primers spanning the Cas9-single-guide RNA cleavage site of exon 2 of the GR gene: exon 2 forward primer, GGACTCCAAAGAATCATTAACTCCTGG (SEQ ID NO:10); exon 2 reverse primer, AATTACCCCAGGGGTGCAGA (SEQ ID NO:11). DNA bands were separated by agarose gel electrophoresis prepared with SYBR-safe DNA gel stain in 0.5x Tris/Borate/EDTA. Gel images were obtained using GeneSys software in a G:BOX gel documentation system (Syngene).

### Western Blot

To detect GR protein expression, CTLs were lysed in lysis buffer (IP Lysis Buffer; Pierce Biotechnology Inc) supplemented with protease inhibitors (Complete Mini, EDTA-free Cocktail tablets; Roche Holding) and incubated for 30 minutes on ice. Protein concentration was determined by the bicinchoninic acid (BCA) assay (Pierce Biotechnology Inc). The following primary antibodies were used: GR (clone D6H2L) XP rabbit monoclonal antibody and β-actin antibody (clone 8H10D10); both antibodies were obtained from Cell Signaling Technology. Blots were imaged using a G:BOX gel documentation system and GeneSys software (Syngene).

### Results

### Expansion of SARS-CoV-2 Reactive T Cells From COVID-19-Recovered Donors

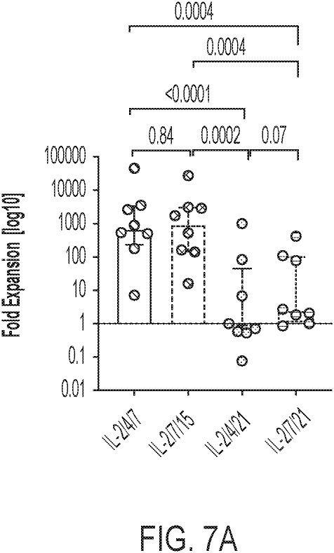

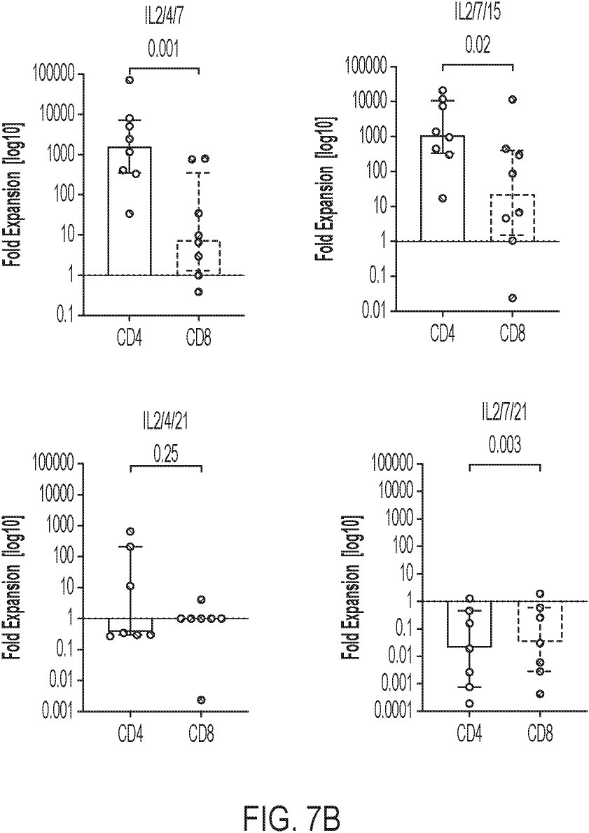

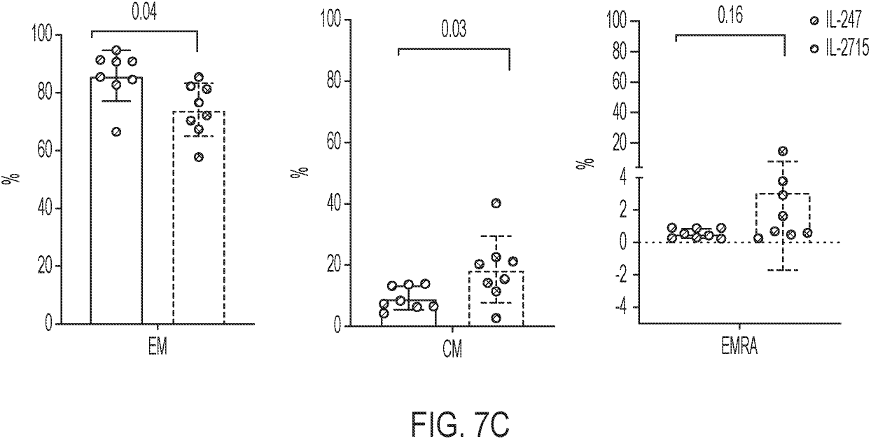

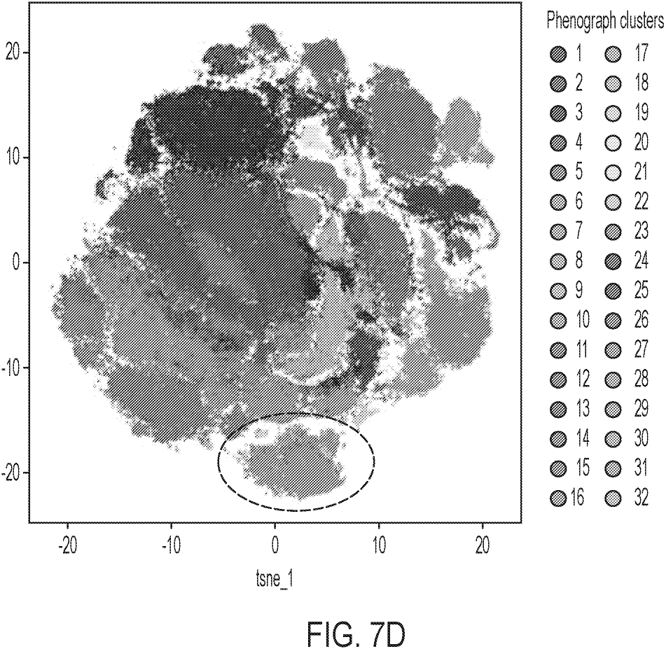

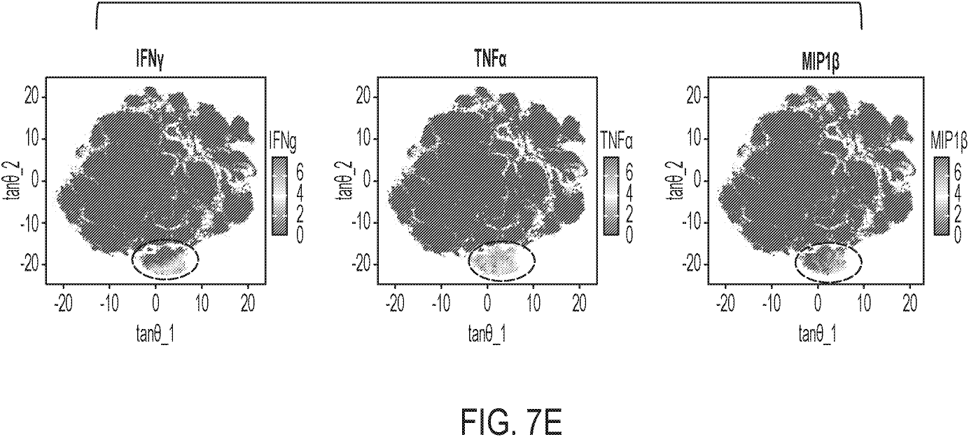

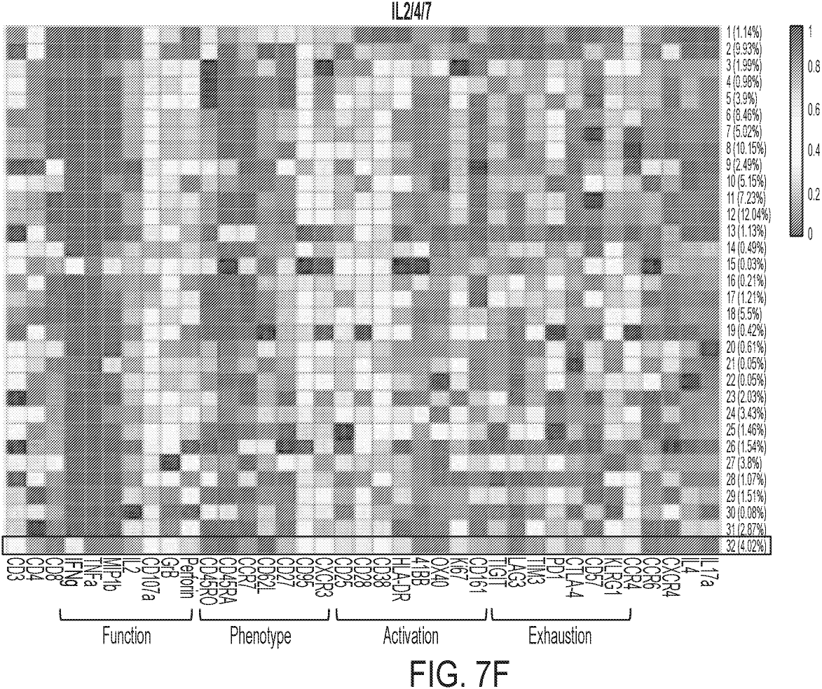

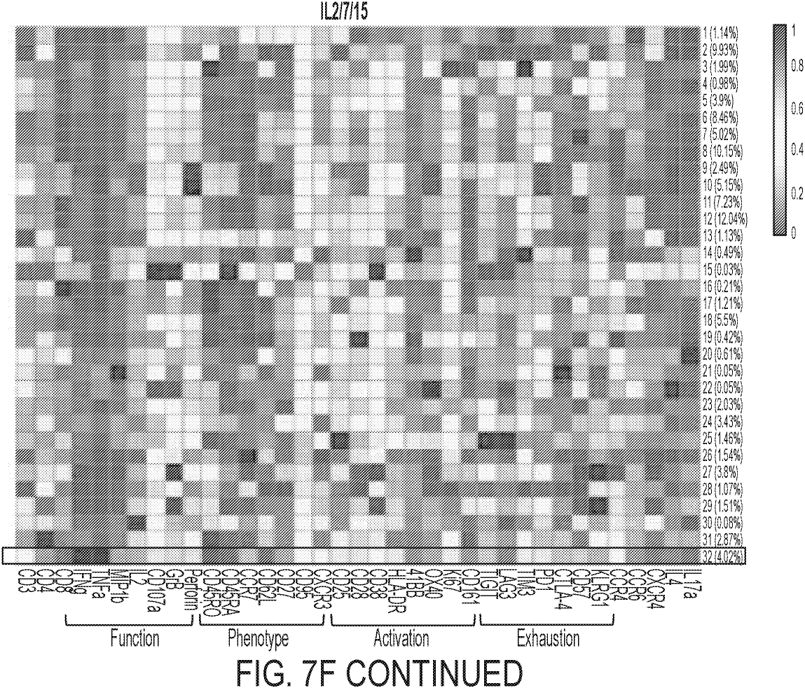

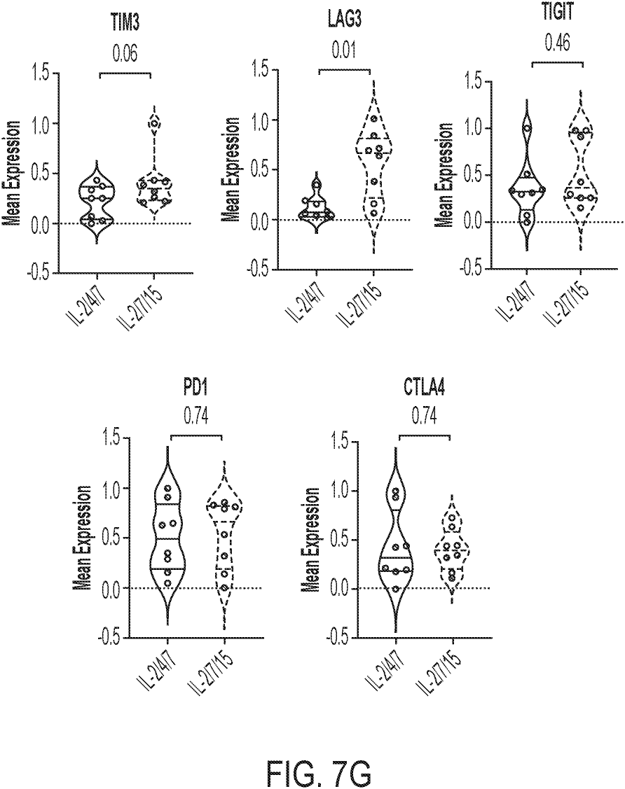

The feasibility of generating VSTs from the peripheral blood (PB) of healthy donors for ACT had been reported.(Muftuoglu et al., 2018) Here, the inventors utilized this approach to derive and expand SARS-CoV-2 specific T-cells. Briefly, PBMCs from 10 CoV-RD were cultured with 11 different peptide libraries (15 mers overlapping by 11 amino acids) spanning the entire sequence of the SARS-CoV-2 antigens, including both the structural (S, M, N, E) and non-structural proteins (AP3A, Y14, NS6, NS7a, NS7B, NS8, ORF9B and ORF10) in the presence of either IL-2/4/7, IL-2/7/15, IL-2/4/21 or IL-2/7/21 for 14 days. At the end of the culture period, SARS-CoV-2 reactive T-cells were enumerated based on their ability to produce IFNy in response to ex vivo stimulation with the viral antigens. When cultured in the presence of IL-2/4/7 or IL-2/7/15, expansion was successful in 8/10 cases, with a median fold expansion of 719.14 (range 7.16 - 45572.50) and 1138.41 (range 15.97 - 27716.61), respectively. However, expansion using IL-2/4/21 or IL-2/7/21 was suboptimal, with a median fold expansion of only 0.71 (range 0.08 - 996.18) and 2.72 (range 0.85 - 415.98), respectively (FIG. 7A, Tables 1, 4 and 5). IL-2/4/7 and IL-2/7/15 culture conditions supported expansion of both CD4+ and CD8+ SARS-CoV-2 specific T-cells with a predominance of CD4+ T-cells, while expansion with IL-2/4/21 and IL-2/7/21 failed to result in significant expansion of either SARS-CoV-2 CD4+ or CD8+ T cells (FIG. 7B).

### SARS-CoV-2 Reactive T Cells Generated From CoV-RD Are Polyfunctional

The functional phenotype of the ex vivo expanded SARS-CoV-2 CTLs was next interrogated. Since IL-2/4/7 and IL-2/7/15 resulted in the best cell expansion, the inventors focused analysis on SARS-CoV-2 CTLs generated using these two conditions. Both culture conditions supported expansion of effector memory (EM) and central memory (CM) T cells although the use of IL-2/7/15 resulted in expansion of a larger proportion of CM T cells (FIG. 7C).

Previous studies in patients with severe COVID-19 have reported the presence of T cells with an exhausted phenotype and reduced polyfunctionality.(Chen et al., 2020; Zheng et al., 2020a, 2020b) Thus, the inventors performed a comprehensive single cell analysis of expanded SARS-CoV-2 CTLs from 8 recovered donors using mass cytometry. Phenotypic interrogation of SARS-CoV-2 reactive T-cells expanded with IL-2/4/7 or IL-2/7/15 (identified based on their ability to produce IFNγ in response to ex vivo stimulation with a mixture of S, M and N peptide libraries) revealed that SARS-CoV-2 specific CTLs are polyfunctional based on their ability to secrete multiple cytokines and chemokines simultaneously, including IFNγ, TNFα and MIP1β (cluster 32; FIGS. 7D and 7E). Moreover, ex vivo expanded SARS-CoV-2 CTLs did not express high levels of inhibitory/checkpoint molecules thus, arguing against an exhausted phenotype (cluster 32; FIG. 7F). Indeed, analysis of functional markers revealed a cytotoxic Th1 phenotype, characterized by expression of IFNγ, TNFα, CD107a and granzyme B (GrB), indicating direct antiviral killing capacity (FIG. 7F). Interestingly, they did not produce significant amounts of IL-2 in response to antigenic stimulation. Single cell phenotypic comparison of SARS-CoV-2 CTLs expanded using the two different culture conditions did not reveal major differences in the expression patterns of activation and functional markers between these two groups (FIG. 7F). However, cells expanded in the presence of IL-2/4/7 expressed lower levels of some exhaustion markers such as TIM3 and LAG3 compared to cells expanded with IL-2/7/15 (FIG. 7G). Taken together, these data support the notion that polyfunctional, non-exhausted T cells capable of reacting against SARS-CoV-2 antigens can be expanded from the PB of CoV-RDs.

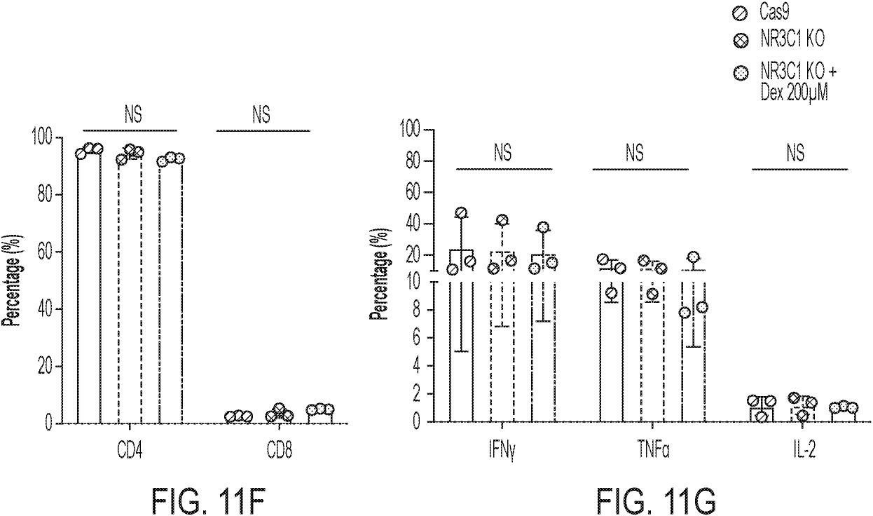

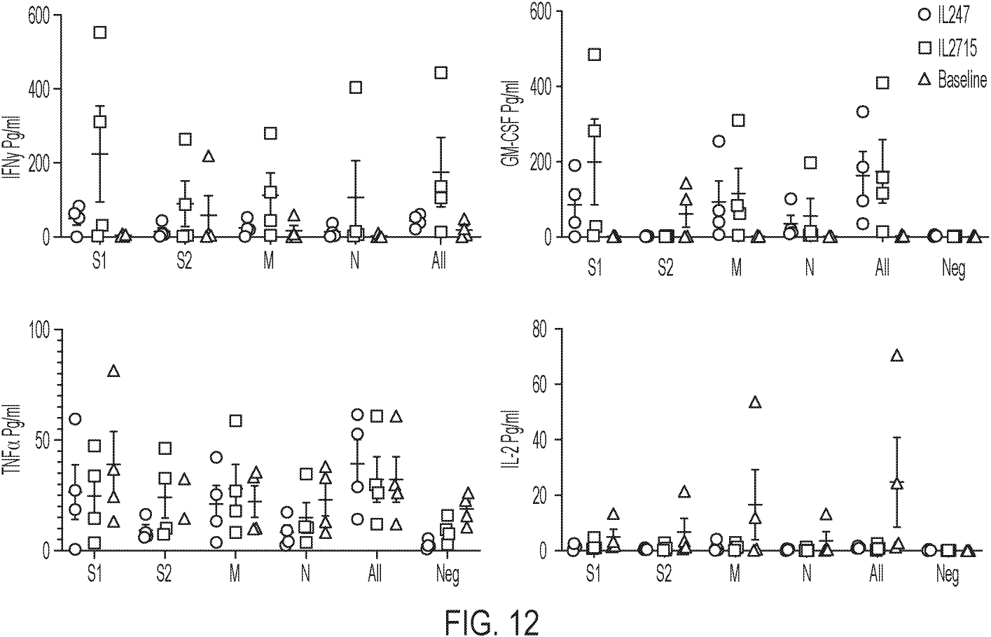

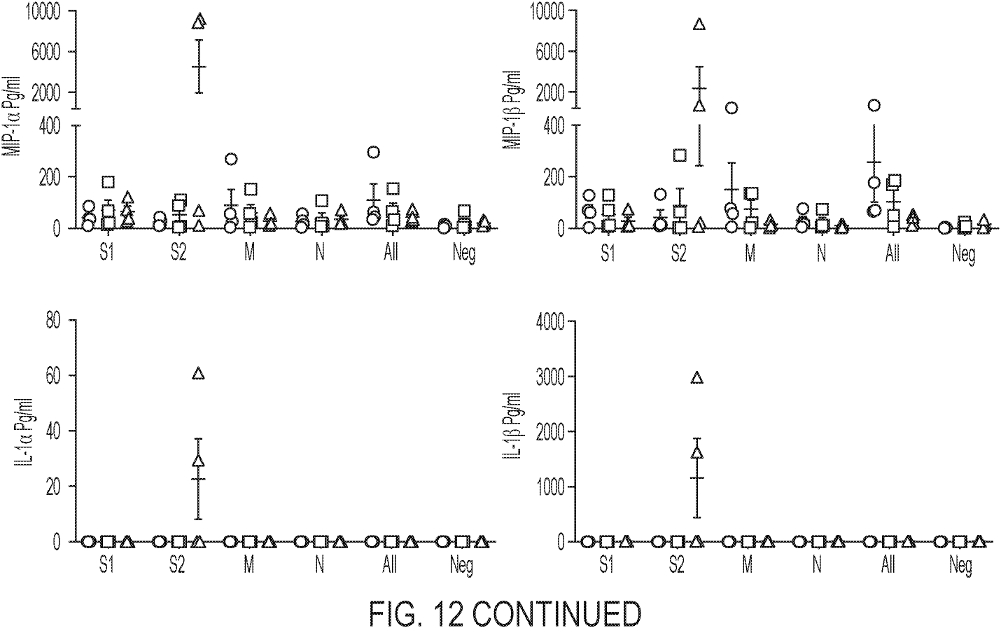

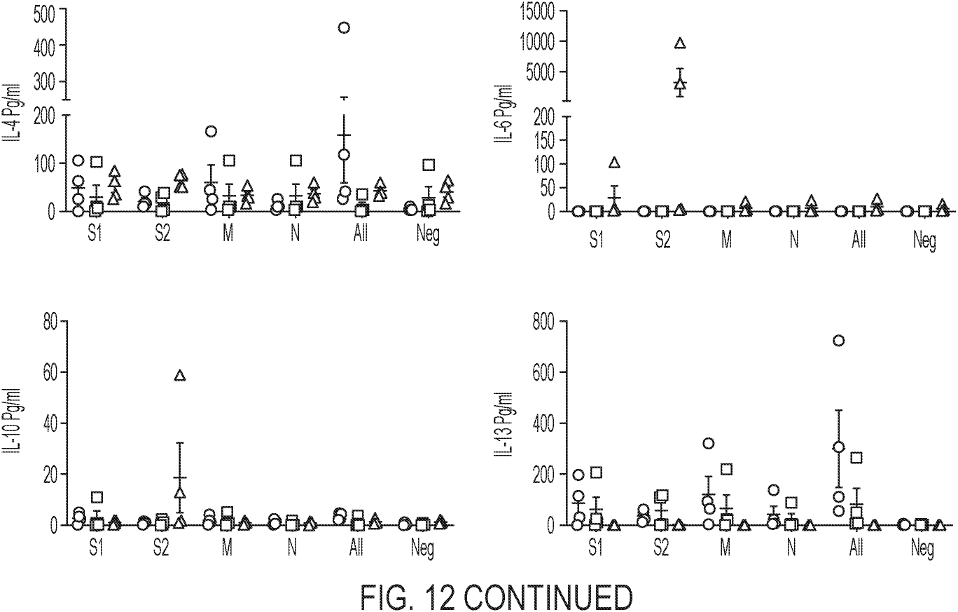

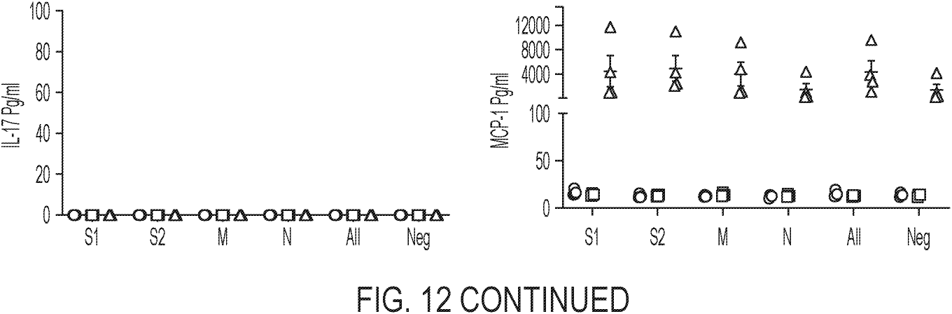

A multiplex analysis was also performed to measure cytokines in supernatants collected from cultures of SARS-CoV-2 CTLs with SARS-CoV-2 antigens (n= 4 for each of the culture conditions- IL-2/4/7 and IL-2/715). As expected, the expanded SARS-CoV-2 CTLs released effector cytokines such as IFNγ, TNFα, MIP1β in response to antigenic stimulation; however, they did not produce cytokines such as IL-6, IL-1α, or IL-10 that could contribute to a higher risk of toxicity or cytokine release syndrome (CRS) (FIG. 12).

### Expanded SARS-CoV-2 CTLs From CoV-RD are Directed Against Structural Proteins, Including Both the C and N Terminals of the S Protein

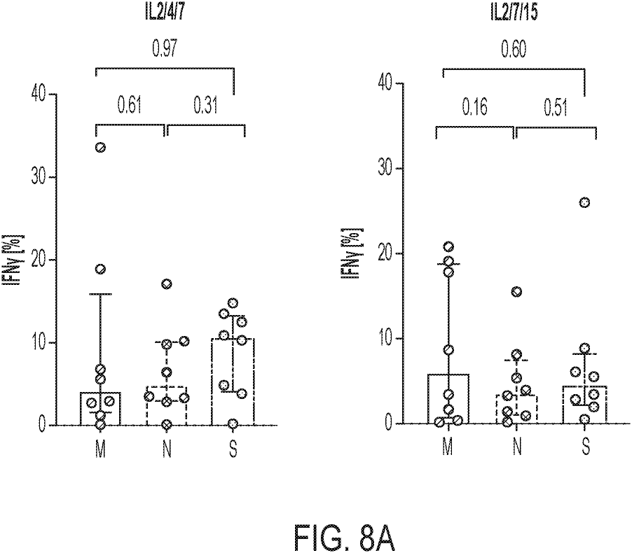

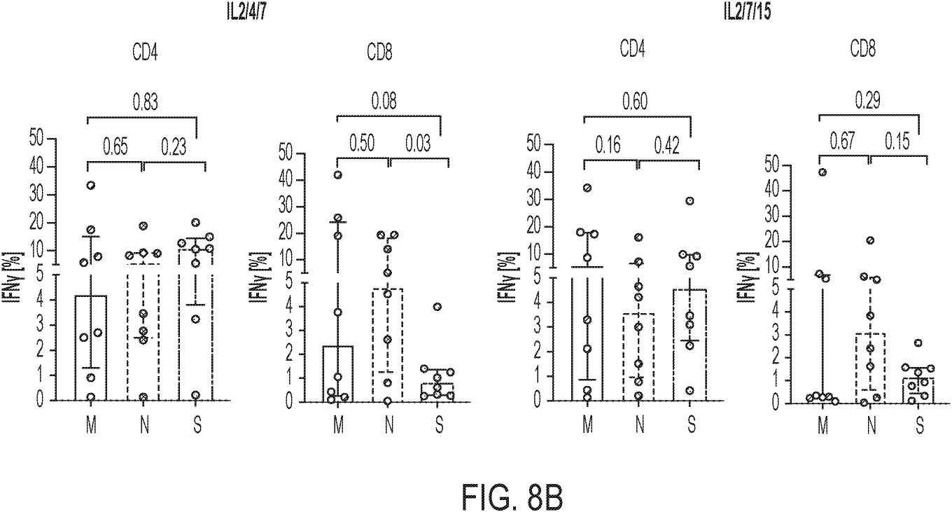

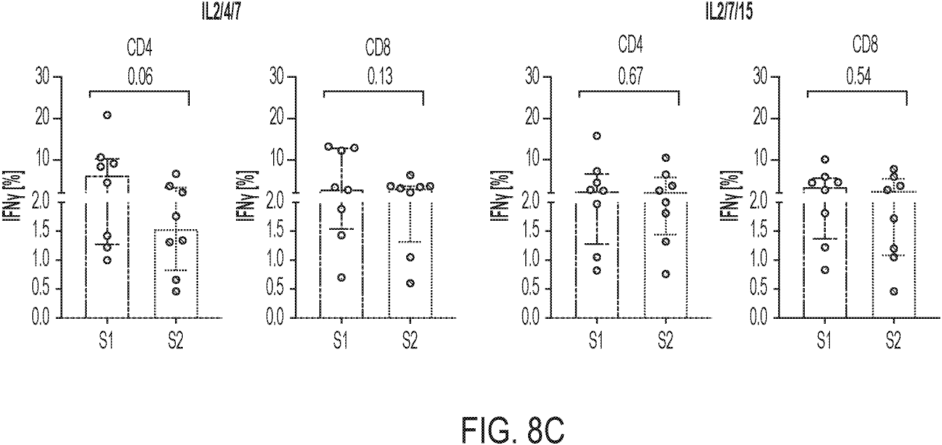

In order to identify the dominant antigen(s) driving expansion of SARS-CoV-2 CTLs, the expanded cells were stimulated ex vivo with peptide libraries derived from either M, N, S or E (structural proteins) or AP3A, Y14, NS6 NS7a, NS7B, NS8, ORF9B or ORF10 (non-structural proteins). Analysis of IFNy production showed that for the lines expanded with IL-2/4/7, the overall T-cell response was mostly directed against S (median 10.60%, range 0.21 -14.8%), with the remaining cells responding to M (median 4.27%, range 0.11- 33.6%) or N (median 4.98%, range 0.12 -17.10%) (FIG. 8A). For the lines expanded with IL-2/7/15, the CD3+ T-cell response favored M (median 6.05%, range 0.15 - 20.80%) followed by S (median 4.47%, range 0.48 - 26.00%) and N (median 3.60 %, range 0.17-15.50%) (FIG. 8A). Interestingly, the inventors also found a correlation between the baseline spike IgG levels In sum, for both IL-2/4/7 and IL-2/7/15 culture settings, seven lines were directed against M + N + S, one line was directed against S + N, and no line reacted to M and N in the absence of S. There was no significant expansion of CTLs in response to the non-structural proteins or the structural E protein.

When the CD4+ and CD8+ T cell responses were considered separately, the response of CD4+ T cells to individual SARS-CoV-2 antigens followed a pattern similar to that observed in the overall CD3+ T cell population. Interestingly, however, CD8+ T cell responses were mostly directed against the N protein, irrespective of the cytokine cocktail used for SARS-CoV-2 CTL expansion (FIG. 8B, Table 1).

Peptides derived from the C-terminus of the S protein have higher homology with the S glycoprotein of human endemic “common cold” coronaviruses; in contrast, the N-terminus of the S protein includes peptides from the receptor-binding domain (the target of neutralizing antibodies) that are more specific to SARS-CoV-2 (Braun et al., 2020; Walls et al., 2020). The expanded CD4+ and CD8+ SARS-CoV-2 CTLs were capable of reacting to both N- and C-terminal epitopes (Pools 1 and 2 of the S protein respectively), indicating their specificity for the receptor binding domain (RBD) of SARS-CoV-2 (FIG. 8C, Tables 2, 6 and 7).

### Culture With IL-2/4/7 Results in Preferential Expansion of T Cells Against the N-Terminus of the S Protein

Since the choice of cytokines used to expand SARS-CoV-2 T-cells modulates the hierarchy of antigenic response, the inventors compared the T cell response against S (S1 and S2), M and N proteins of SARS-CoV-2 in PB samples at baseline (prior to expansion) with that observed in paired expanded CTLs from Cov-RDs.

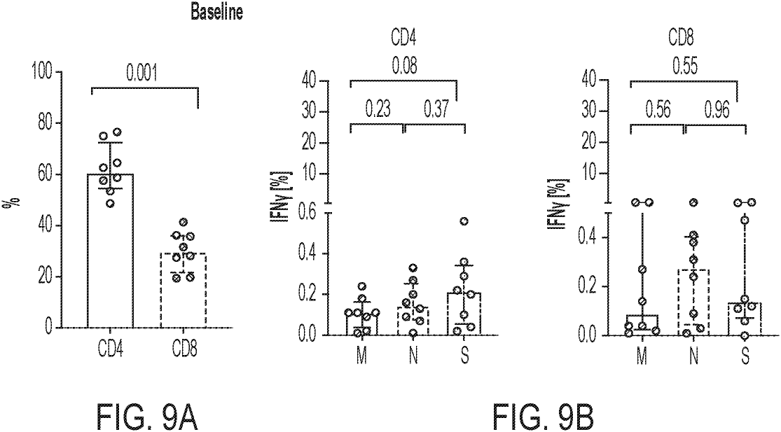

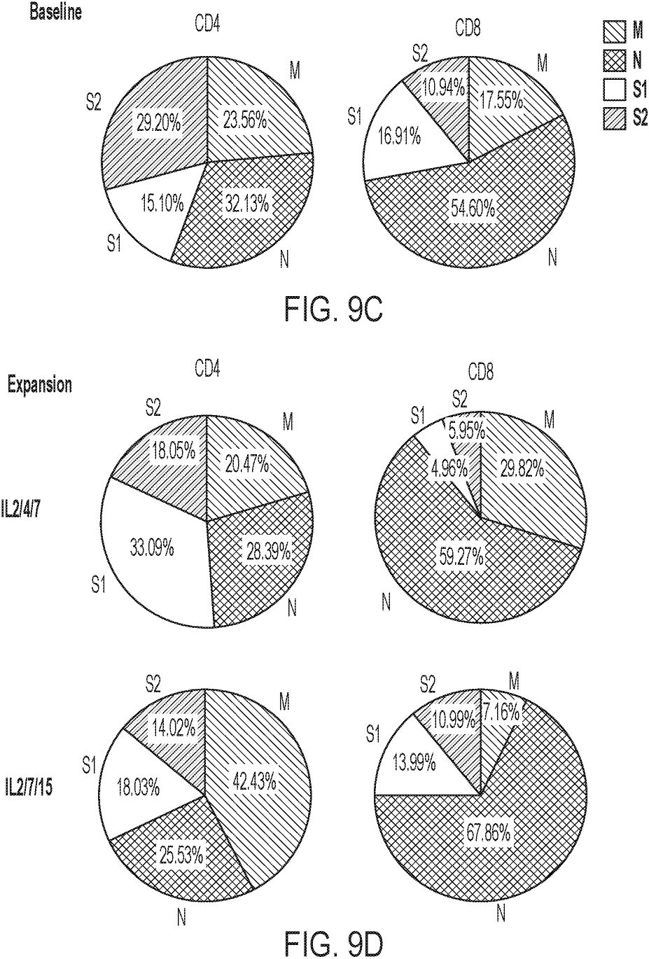

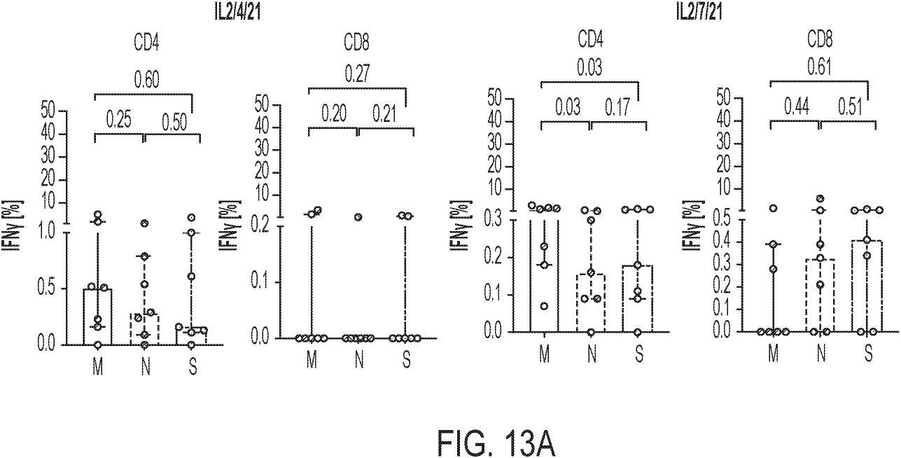

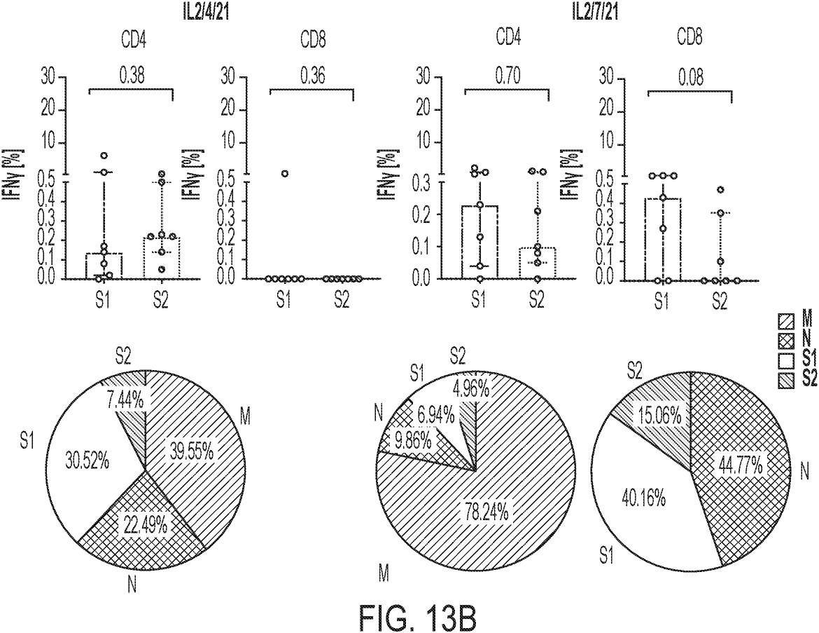

At baseline (pre-expansion), the responses were mostly CD4 dominant (FIG. 9A), and directed against the S protein (median 0.21%, range 0.02%-0.56%), followed by N (median 0.15%, range 0.01-0.33%) and M (median 0.11%, range 0.01-0.24%) (FIG. 9B), indicating an immunogenic dominance for S protein HLA class II epitopes. There were not measurable responses against non-structural proteins (data not shown). Following expansion, culture with IL-2/4/7 maintained the hierarchy of CD4+ T cell response toward S protein, (FIGS. 8B, 9C, and 9D), with a greater proportion of T cells directed against S1, while culture with IL-2/7/15 favored a response toward M protein (FIGS. 8B, 9C, and 9D). Expansion with IL-2/4/21 and IL-2/7/21 yielded very low numbers of CTLs, most of which were CD4+. Interestingly, the pattern of antigenic response for cells cultured using these two conditions differed from that observed for IL-2/4/7 and was similar to IL-2/7/15, in that the majority of CD4+ CTLs reacted instead to M and N proteins (FIG. 13A). For CTLs that did show a response to S protein, further analysis detected no particular pattern of reactivity to either terminus of the protein (FIG. 13B).

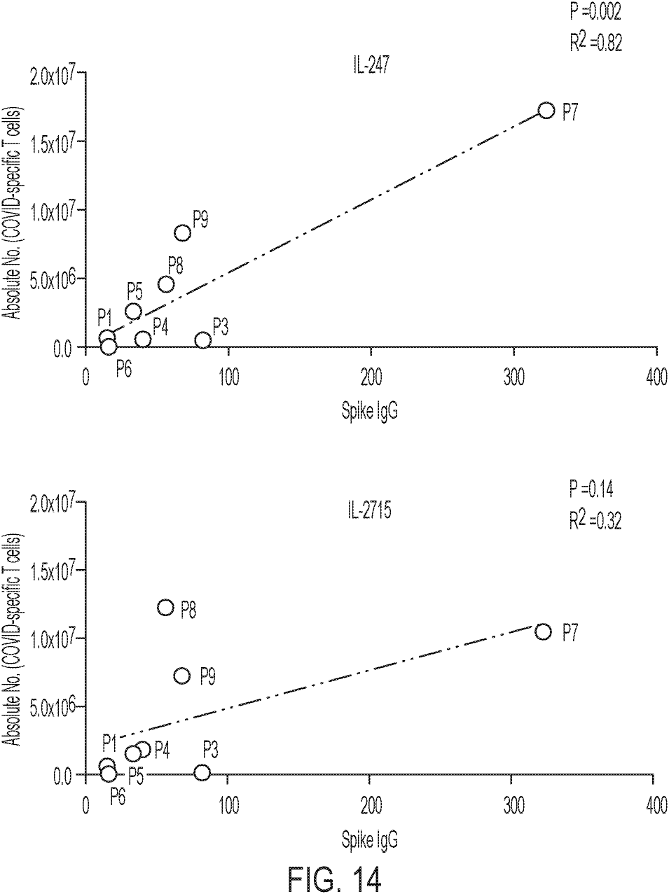

Furthermore, there was a significant correlation (p=0.002, R²=0.82) between the spike protein IgG antibody titer measured in plasma from the recovered donors and the absolute number of SARS-CoV-2 specific T cells following expansion with IL-2/4/7, but not with the IL-2/7/15 cytokine cocktail (FIG. 14).

These data indicate that the antigenic skewing can be driven both by the immunodominance of the protein and by the culture conditions and support the use of IL-2/4/7 for the expansion of SARS-CoV-2 T-cells for clinical use.

### SARS-CoV-2 T-Cells Can be Expanded From the PB of Healthy Donors but at Lower Frequencies Than for Cov-RD Donors

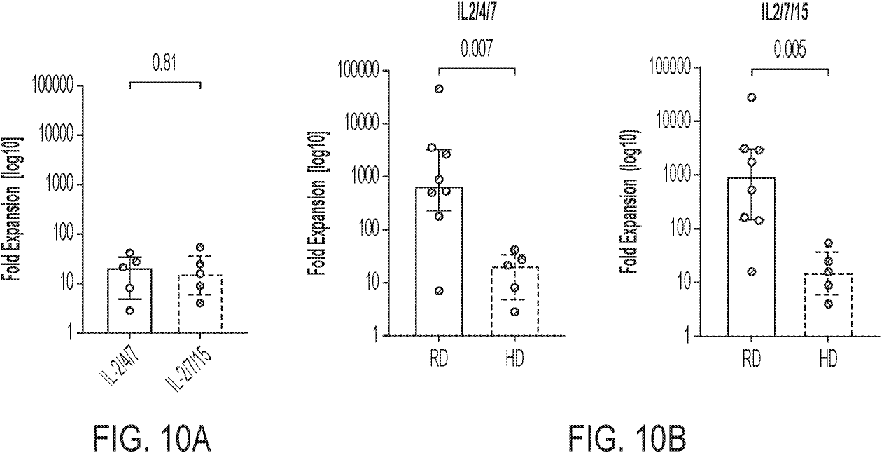

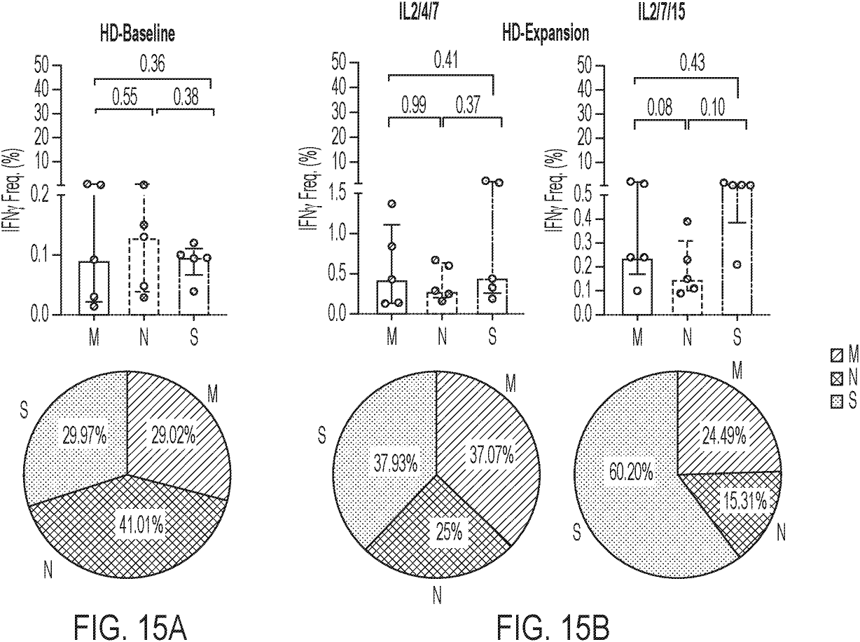

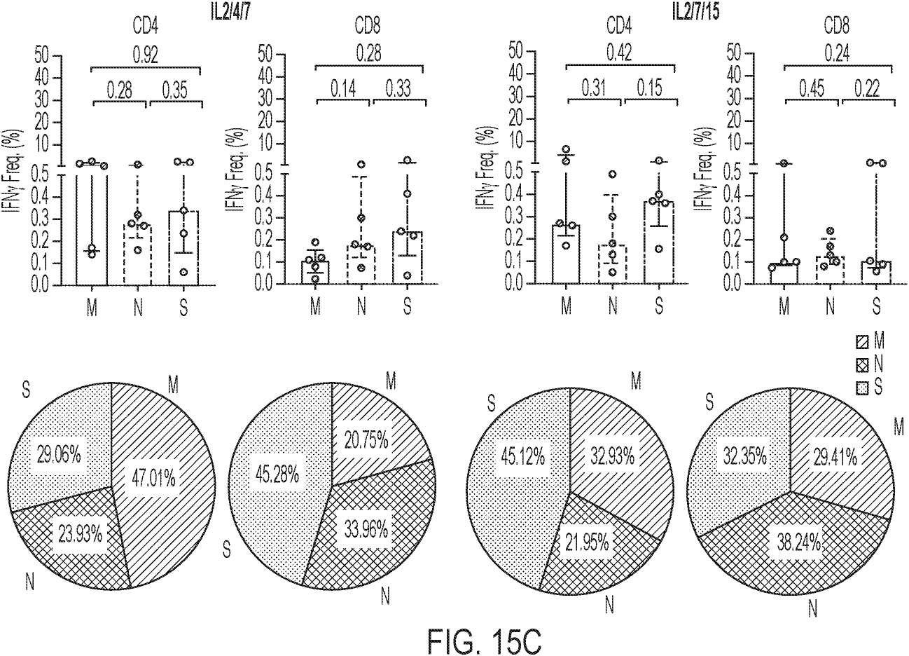

Recent reports indicate the presence of SARS-CoV-2 T cells in the PB of healthy donors (HD) not exposed to COVID-19(Braun et al., 2020; Pia, 2020). Thus, it was considered if SARS-CoV-2 T-cells can be expanded from the PB of HD and whether they have a similar pattern of SARS-CoV-2 recognition as those generated from Cov-RDs. PBMC from 5 HDs were expanded using the same protocol as for Cov-RDs. Only a modest expansion of CTLs recognizing SARS-CoV-2 antigens over a 14-day culture period was achieved, with a median 20.37-fold increase (range, 2.85 - 41.84) for IL-2/4/7 culture condition and 21.49-fold increase (range, 4.00 - 53.95) for IL-2/7/15 culture condition (FIG. 10A). The frequencies of SARS-CoV-2 CTLs from the PB of healthy donors after 14 days of culture were significantly lower than those achieved with PB from Cov-RDs (FIG. 10B). At baseline (pre-expansion), assessment of IFNy production and CTL frequencies suggested that in healthy donors responses were directed mostly against the N protein (median 0.13%, range 0.03%-0.37%), followed by S (median 0.10%, range 0.04-0.12%) and M (median 0.09%, range 0.01-0.75%) (FIG. 15A). Culture in the presence of S protein could skew the response towards S, albeit at much lower frequencies than that observed with Cov-RDs (FIG. 15B, Tables 8 and 9). The inventors did not find any particular pattern of antigenic response in the CD4 and CD8 compartments (FIG. 15B, Tables 8 and 9). Expansion was not successful in IL-2/4/21 or IL-2/7/21 stimulation conditions.

### Expanded COVID-19 Specific T Cells Can Be Genetically Modified to Render Them Steroid-Resistant

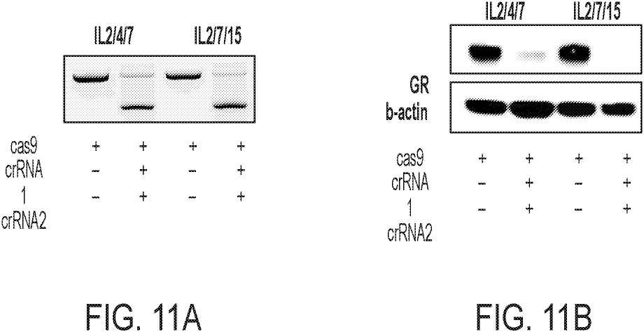

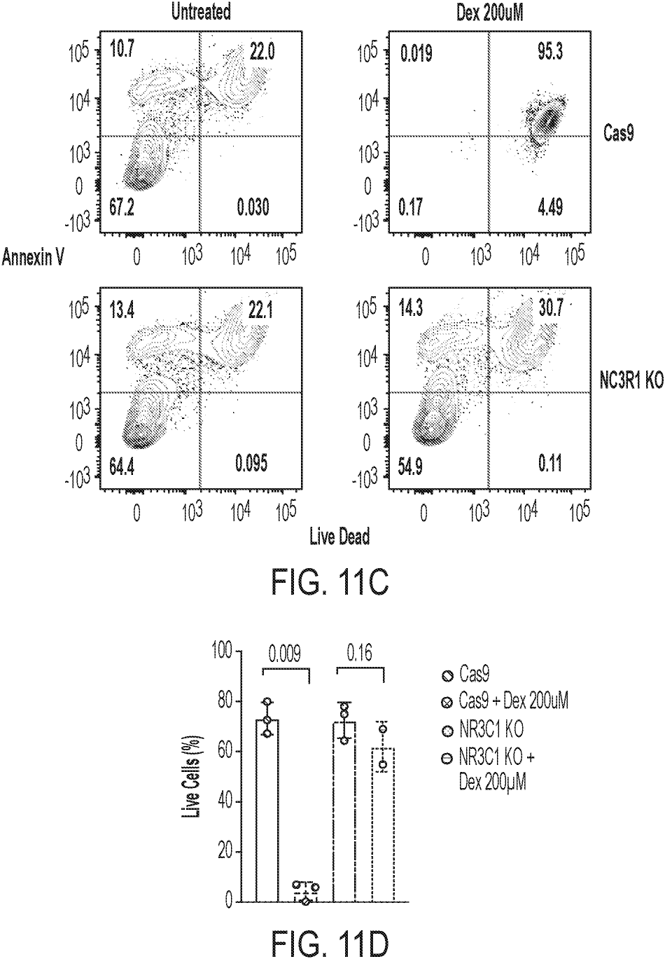

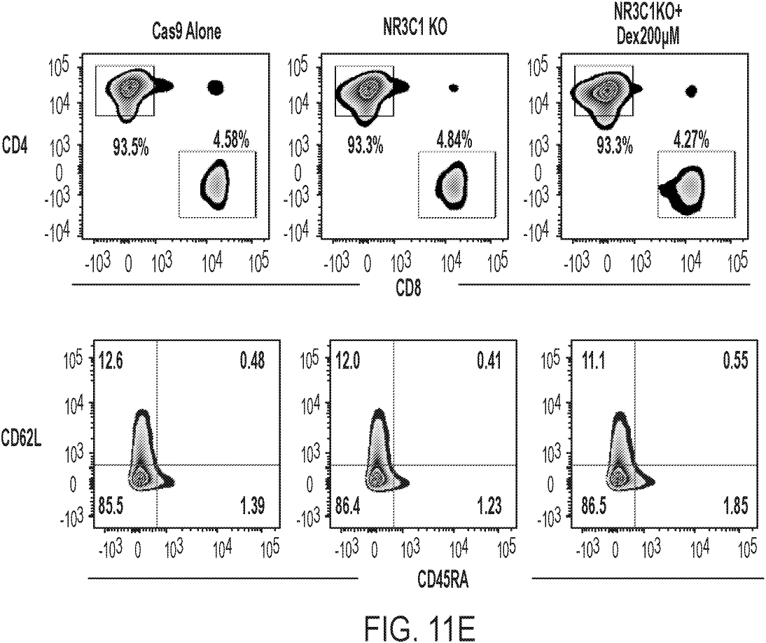

Corticosteroids are used in the treatment of patients with COVID-19-related ARDS to reduce mortality associated with this condition. SARS-CoV-2 specific T-cell therapy is not an option in such patients as corticosteroids induce apoptosis of adoptively transferred T cells, thus, significantly limiting the efficacy of this approach. To address this challenge, CRISPR CAS 9 gene editing was used to knockout the glucocorticoid receptor (NR3C1) in SARS-CoV-2 CTLs and confirmed high efficiency of deletion (>90%) as determined by PCR and western blot analysis (FIGS. 11A and 11B). Annexin V apoptosis assay confirmed that the viability of NR3C1 KO CTLs treated with dexamethasone was similar to that of control CTLs (defined as CTLs electroporated with Cas9 alone) (FIGS. 11C and 11D). Moreover, NR3C1 KO SARS-CoV-2 CTLs maintained similar phenotype and distribution of CD4+ and CD8+ T cell subsets when compared to control SARS-CoV-2 CTLs and retained their effector functions (FIGS. 11E - 11G).

In particular embodiments, the present disclosed ACT for COVID-19 is unlikely to worsen cytokine release syndrome (CRS) as the adoptively infused CTLs will target and kill the SARS-CoV-2 infected myeloid cells, thus breaking the vicious cycle driving the cytokine storm. Furthermore, CRS is not unique to beta-coronavirus infections and has been reported with other viral infections such as CMV, EBV and adenovirus (Humar et al., 1999; McLaughlin et al., 2018; Ramos-Casals et al., 2014) where adoptive cell therapy with virus-specific CTLs has been used to treat hundreds of patients with severe infections effectively and with minimal complications (Bollard and Heslop, 2016; McLaughlin et al., 2018; Muftuoglu et al., 2018; Tzannou et al., 2017).

The presently disclosed methods and compositions allow for cryopreservation and banking of SARS-CoV-2 T-cells, facilitating the rapid identification and selection of viral-specific T-cells for ACT based on the most closely HLA-matched third-party donor as published by the inventors and others for other severe viral infections (Eiz-Vesper et al., 2012; Haque et al., 2007; Leen et al., 2011; Muftuoglu et al., 2018; O’Reilly et al., 2016). An additional advantage to the approach is that the genetic modification of SARS-CoV-2 T-cells to delete the glucocorticoid receptor allows treatment of patients with ARDS on high doses corticosteroids.

### Example 4

### Production of Covid-19 Specific T Cells That Target SARS-CoV-2 Variants

Covid-19 specific T cells were generated that are able to target presently emerging variants of SARS-CoV-2.

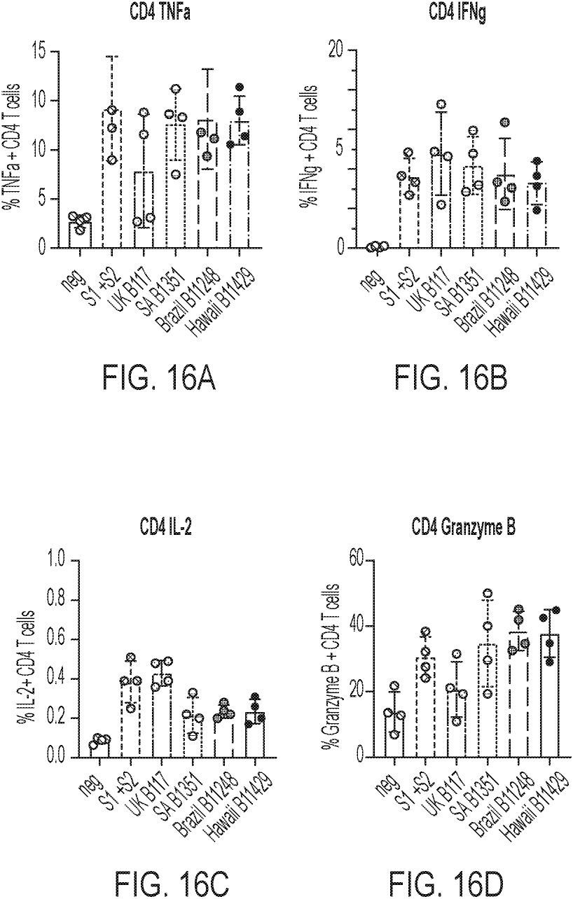

SARS-CoV-2 CTLs were generated by first incubating PBMC with 1 µg of membrane, nucleocapsid and spike pepmixes for 2 hours, and then they were expanded in complete Click’s media supplemented with IL-2 (20 IU/ml), IL-4 (60 ng/ml) and IL-7 (10 ng/ml) for 14 days to generate COVID-19 specific T cells. Intracellular staining was performed to assess TNFα, IFNγ, IL-2 and Granzyme B production from COVID-19 specific T cells that were exposed to spike peptides (1 µg) from the original strain or from emerging variants (B117, B1351, B11248, and B11249) for 6 hours. Bar graphs represent the percentage of TNFα (FIG. 16A), IFNγ (FIG. 16B), IL-2 (FIG. 16C), and granzyme B (FIG. 16D) produced by the CD4+ fraction of the COVID-19 specific T cells. As shown therein, CD4 CTLs produce these cytokines in response to exposure to COVID19 variants.

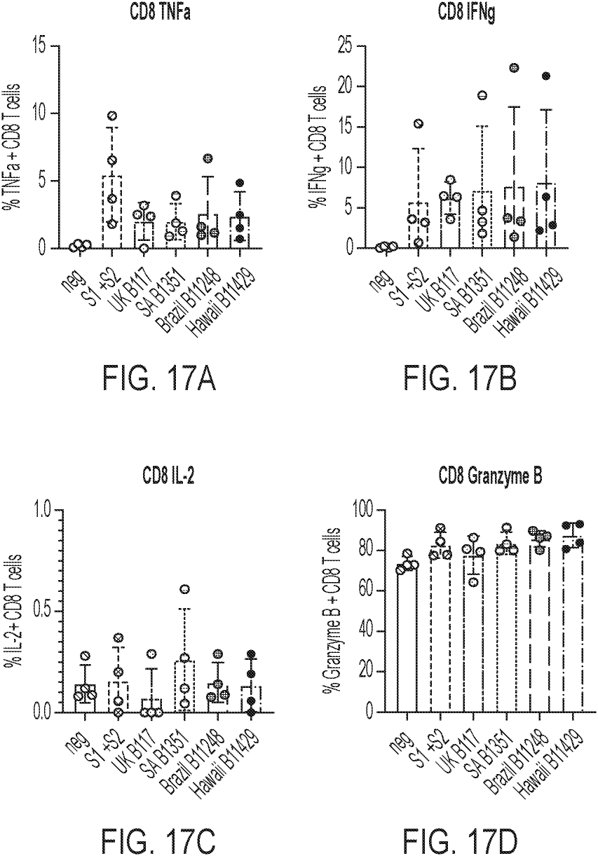

In addition, SARS-CoV-2 CTLs were generated by first incubating PBMC with1µg of membrane, nucleocapsid and spike pepmixes for 2 hours, followed by expansion in complete Click’s media supplemented with IL-2 (20 IU/ml), IL-4 (60 ng/ml) and IL-7 (10 ng/ml) for 14 days to generate COVID-19 specific T cells. Intracellular staining was performed to assess TNFα, IFNγ, IL-2 and granzyme B production from COVID-19 specific T cells that were exposed to spike peptides (1 µg) from the original strain or from the emerging variants (B117, B1351, B11248, and B11249) for 6 hours. Bar graphs represent the percentage of TNFα (FIG. 17A), IFNγ (FIG. 17B), IL-2 (FIG. 17C), and granzyme B (FIG. 17D) produced by the CD8+ fraction of the COVID-19 specific T cells. CD8 CTLs also produce cytokines in response to exposure to COVID19 variants.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

Basar, R., Daher, M., Uprety, N., Gokdemir, E., Alsuliman, A., Ensley, E., Ozcan, G., Mendt, M., Hernandez Sanabria, M., Kerbauy, L.N., et al. (2020). Large-scale GMP-compliant CRISPR-Cas9-mediated deletion of the glucocorticoid receptor in multivirus-specific T cells. Blood Adv. 4, 3357-3367.

Bollard, C.M., and Heslop, H.E. (2016). T cells for viral infections after allogeneic hematopoietic stem cell transplant. Blood 127, 3331-3340.

Braun, J., Loyal, L., Frentsch, M., Wendisch, D., Georg, P., Kurth, F., Hippenstiel, S., Dingeldey, M., Kruse, B., Fauchere, F., et al. (2020). Presence of SARS-CoV-2 reactive T cells in COVID-19 patients and healthy donors. MedRxiv 2020.04.17.20061440.

Chen, G., Wu, D., Guo, W., Cao, Y., Huang, D., Wang, H., Wang, T., Zhang, X., Chen, H., Yu, H., et al. (2020). Clinical and immunological features of severe and moderate coronavirus disease 2019. J. Clin. Invest. 130, 2620-2629.

Chothia et al., The outline structure of the T-cell alpha beta receptor. EMBO J. 7:3745-3755, 1988.Christensen JP, Doherty PC, Branum KC, Riberdy JM. Profound protection against respiratory challenge with a lethal H7N7 influenza A virus by increasing the magnitude of CD8(+) T-cell memory. Journal of virology 2000; 74(24): 11690-6.

Davila et al. CD19 CAR-targeted T cells induce long-term remission and B Cell Aplasia in an immunocompetent mouse model of B cell acute lymphoblastic leukemia. PLoS ONE 8(4): e61338, 2013.

Eichelberger M, Allan W, Zijlstra M, Jaenisch R, Doherty PC. Clearance of influenza virus respiratory infection in mice lacking class I major histocompatibility complex-restricted CD8+ T cells. J Exp Med 1991; 174(4): 875-80.

Eiz-Vesper, B., Maecker-Kolhoff, B., and Blasczyk, R. (2012). Adoptive T-cell immunotherapy from third-party donors: Characterization of donors and set up of a T-cell donor registry. Front. Immunol. 2012; 3: 410.

Fedorov et al., PD-1- and CTLA-4-Based Inhibitory Chimeric Antigen Receptors (iCARs) Divert Off-Target Immunotherapy Responses, Sci Transl Med. 2013 Dec 11; 5(215): 215ral72.

Haque, T., Wilkie, G.M., Jones, M.M., Higgins, C.D., Urquhart, G., Wingate, P., Burns, D., McAulay, K., Turner, M., Bellamy, C., et al. (2007). Allogeneic cytotoxic T-cell therapy for EBV-positive posttransplantation lymphoproliferative disease: Results of a phase 2 multicenter clinical trial. Blood. vol. 110, no. 4, pp. 1123-31.

Heemskerk et al. Adoptive cell therapy for patients with melanoma, using tumor-infiltrating lymphocytes genetically engineered t secrete inerleukin-2. Hum Gene Ther. 19:496-510, 2008.

Humar, A., St Louis, P., Mazzulli, T., McGeer, A., Lipton, J., Messner, H., and MacDonald, K.S. (1999). Elevated serum cytokines are associated with cytomegalovirus infection and disease in bone marrow transplant recipients. J Infect Dis 179, 484-488.

Johnson et al. Gene therapy with uman and mouse T-cell receptors mediates cancer regression and targets normal tisues expressing conate antigen. Blood 114:535-46, 2009.

Jores et al., PNAS U.S.A. 87:9138-9142, 1990.

Kabat et al., “Sequences of Proteins of Immunological Interest, US Dept. Health and Human Services, Public Health Service National Institutes of Health, 1991, 5^{th} ed.)

Kong SL, Chui P, Lim B, Salto-Tellez M. Elucidating the molecular physiopathology of acute respiratory distress syndrome in severe acute respiratory syndrome patients. Virus Res 2009; 145(2): 260-9.

Leen, A.M., Bollard, C.M., Mendizabal, A., Shpall, E.J., Szabolcs, P., Antin, J.H., Kapoor, N., Pai, S.-Y., Grilley, B., Gee, A.P., et al. (2011). Most Closely HLA-Matched Allogeneic Virus Specific Cytotoxic T-Lymphocytes (CTL) to Treat Persistent Reactivation or Infection With Adenovirus, CMV and EBV After Hemopoietic Stem Cell Transplantation (HSCT). Biol. Blood Marrow Transplant.

Lefranc et al., IMGT unique numbering for immunoglobuling and T cell receptor variable domains and Ig superfamily V-like domains. Dev. Comp. Immunol. 27:55, 2003.

Li, et al., Directed evolution of human T-cell receptors with picomolar affinities by phage display. Nat Biotechnol. 2005 Mar;23(3):349-542005.

Libraty DH, O’Neil KM, Baker LM, Acosta LP, Olveda RM. Human CD4(+) memory T-lymphocyte responses to SARS coronavirus infection. Virology 2007; 368(2): 317-21.

McLaughlin, L.P., Bollard, C.M., and Keller, M.D. (2018). Adoptive T Cell Therapy for Epstein-Barr Virus Complications in Patients With Primary Immunodeficiency Disorders. Front. Immunol. 9, 556.

Muftuoglu, M., Olson, A., Marin, D., Ahmed, S., Mulanovich, V., Tummala, S., Chi, T.L., Ferrajoli, A., Kaur, I., Li, L., et al. (2018). Allogeneic BK Virus-Specific T Cells for Progressive Multifocal Leukoencephalopathy. N Engl J Med 379, 1443-1451.

O’Reilly, R.J., Prockop, S., Hasan, A.N., Koehne, G., and Doubrovina, E. (2016). Virus-specific T-cell banks for “off the shelf” adoptive therapy of refractory infections. Bone Marrow Transplant. 2016 Sep;51(9):1163-72.

Park M, Thwaites RS, Openshaw PJM. COVID-19: Lessons from SARS and MERS. European Journal of Immunology 2020; 50(3): 308-11.

Pia, L. (2020). SARS-CoV-2-reactive T cells in patients and healthy donors. Nat. Rev. Immunol. 20, 353.

Ramos-Casals, M., Brito-Zerón, P., López-Guillermo, A., Khamashta, M.A., and Bosch, X. (2014). Adult haemophagocytic syndrome. Lancet 383, 1503-1516.

Sadelain et al., The basic principles of chimeric antigen receptor design. Cancer Discov. 3(4): 388-398, 2013.

Thevarajan I, Nguyen THO, Koutsakos M, et al. Breadth of concomitant immune responses prior to patient recovery: a case report of non-severe COVID-19. Nature Medicine 2020.

Tosato, G, and Cohen, JI, Current Protocols in Immunology, Ed Coligan et al, Wiley, 2007, Chapter 7:Unit 7.22

Turtle et al., Engineered T cells for anti-cancer therapy. Curr. Opin. Immunol., 24(5): 633-39, 2012.

Tzannou, I., Papadopoulou, A., Naik, S., Leung, K., Martinez, C.A., Ramos, C.A., Carrum, G., Sasa, G., Lulla, P., Watanabe, A., et al. (2017). Off-the-Shelf Virus-Specific T Cells to Treat BK Virus, Human Herpesvirus 6, Cytomegalovirus, Epstein-Barr Virus, and Adenovirus Infections After Allogeneic Hematopoietic Stem-Cell Transplantation. J. Clin. Oncol. Off. J. Am. Soc. Clin. Oncol. 35, 3547-3557.

Varela-Rohena et al., Genetic engineering of T cells for adoptive immunotherapy. Immunol Res. 2008;42(1-3):166-81.

Walls, A.C., Park, Y.-J., Tortorici, M.A., Wall, A., McGuire, A.T., and Veesler, D. (2020). Structure, Function, and Antigenicity of the SARS-CoV-2 Spike Glycoprotein. Cell 181, 281-292.e6.

Wong, EC, et al., Cytotherapy, 4: 65-76, 2002

Wu et al., Adoptive T-cell therapy using autologous tumor-infiltrating lymphocytes for metastatic melanoma: current status and future outlook. Cancer, 18(2): 160-75, 2012.

Zhao J, Zhao J, Perlman S. T cell responses are required for protection from clinical disease and for virus clearance in severe acute respiratory syndrome coronavirus-infected mice. Journal of virology 2010; 84(18): 9318-25.

Zheng, H.-Y., Zhang, M., Yang, C.-X., Zhang, N., Wang, X.-C., Yang, X.-P., Dong, X.-Q., and Zheng, Y.-T. (2020a). Elevated exhaustion levels and reduced functional diversity of T cells in peripheral blood may predict severe progression in COVID-19 patients. Cell. Mol. Immunol. 17, 541-543.

Zheng, M., Gao, Y., Wang, G., Song, G., Liu, S., Sun, D., Xu, Y., and Tian, Z. (2020b). Functional exhaustion of antiviral lymphocytes in COVID-19 patients. Cell. Mol. Immunol. 17, 533-535.
U.S. Pat. No. 6,410,319
U.S. Pat. No. 7,446,190
U.S. Pat. No. 6,451,995
U.S. Pat. No. 7,070,995,
U.S. Pat. No. 7,265,209
U.S. Pat. No. 7,354,762
U.S. Pat. No. 7,446,179
U.S. Pat. No. 7,446,190
U.S. Pat. No. 7,446,191
U.S. Pat. No. 8,252,592
U.S. Pat. No. 8,324,353
U.S. Pat. No. 8,339,645
U.S. Pat. No. 8,398,282
U.S. Pat. No. 8,479,118
U.S. Pat.Application Publication US2002/131960,
U.S. Pat. Application Publication US2013/287748
U.S. Pat. Application Publication US2013/0149337
WO2000/14257
WO2012/129514
WO2013/071154
WO2013/123061
WO2013/126726
WO2013/166321
WO2014/031687
WO2014/055668
European Patent Application Number EP2537416

### TABLES

Percent IFNγ, IL-2 and TNFα production (median, minimum and maximum values) from the CD3+, CD4+ and CD8+ compartments of COVID-19 reactive T cells stimulated with the peptide libraries derived from M, N and S structural proteins after expansion under the different culture conditions with the four different cytokine cocktails IL-2/4/7, IL-2/7/15, IL-2/4/21 and IL-2/7/21.

TABLE 1

Cytokine production of COVID-19 reactive T cells from recovered donors expanded with different cytokine cocktails against M, N and S structural proteins

| | | CD3% | | | CD4% | | | CD8% | | |
|---|---|---|---|---|---|---|---|---|---|---|
| | | M | N | S | M | N | S | M | N | S |
| IL-2/4/7 | IFNg median | 4.27 | 4.98 | 10.60 | 4.24 | 5.88 | 10.65 | 2.42 | 4.81 | 0.82 |
| | IFNg min | 0.11 | 0.12 | 0.21 | 0.14 | 0.14 | 0.22 | 0.10 | 0.050 | 0.25 |
| | IFNg max | 33.60 | 17.10 | 14.60 | 33.40 | 16.90 | 20.10 | 42.00 | 19.40 | 3.99 |
| | IL-2 median | 0.68 | 1.01 | 0.96 | 0.41 | 0.70 | 0.90 | 0.42 | 0.42 | 0.27 |
| | IL-2 min | 0.14 | 0.23 | 0.20 | 0.14 | 0.18 | 0.16 | 0.00 | 0.16 | 0.18 |
| | IL-2 max | 1.41 | 1.85 | 2.31 | 1.44 | 2.09 | 1.82 | 1.20 | 0.71 | 0.79 |
| | TNFa median | 2.80 | 2.35 | 3.99 | 2.64 | 2.09 | 4.59 | 1.45 | 0.97 | 0.61 |
| | TNFa min | 0.19 | 0.22 | 0.25 | 0.05 | 0.04 | 0.07 | 0.10 | 0.00 | 0.09 |
| | TNFa max | 12.90 | 7.50 | 7.86 | 12.90 | 8.41 | 7.90 | 5.65 | 3.70 | 2.42 |
| IL-2/7/15 | IFNg median | 6.05 | 3.60 | 4.47 | 6.00 | 3.61 | 4.55 | 0.33 | 3.13 | 1.15 |
| | IFNg min | 0.15 | 0.17 | 0.46 | 0.13 | 0.21 | 0.42 | 0.09 | 0.04 | 0.12 |
| | IFNg max | 20.80 | 15.50 | 26.00 | 34.20 | 16.10 | 29.40 | 47.30 | 20.50 | 2.65 |
| | IL-2 median | 1.03 | 0.53 | 0.79 | 0.68 | 0.58 | 0.80 | 0.37 | 0.49 | 0.41 |
| | IL-2 min | 0.15 | 0.32 | 0.31 | 0.20 | 0.28 | 0.22 | 0.20 | 0.23 | 0.27 |
| | IL-2 max | 2.55 | 1.56 | 6.59 | 1.64 | 0.91 | 6.52 | 2.24 | 1.16 | 1.75 |
| | TNFa median | 4.18 | 1.70 | 2.37 | 3.90 | 1.46 | 2.74 | 0.69 | 1.29 | 0.76 |
| | TNFa min | 0.24 | 0.38 | 0.78 | 0.10 | 0.48 | 0.33 | 0.00 | 0.10 | 0.24 |
| | TNFa max | 7.06 | 4.20 | 10.10 | 12.60 | 4.50 | 11.20 | 18.50 | 7.27 | 3.61 |
| IL-2/4/21 | IFNg median | 0.30 | 0.20 | 0.36 | 0.51 | 0.29 | 0.16 | 0.00 | 0.00 | 0.00 |
| | IFNg min | 0.03 | 0.14 | 0.06 | 0.00 | 0.00 | 0.00 | 0.00 | 0.00 | 0.00 |
| | IFNg max | 2.46 | 0.64 | 2.53 | 4.99 | 1.08 | 3.63 | 3.37 | 0.22 | 0.97 |
| | IL-2 median | 0.16 | 0.21 | 0.16 | 0.13 | 0.20 | 0.16 | 0.00 | 0.00 | 0.00 |
| | IL-2 min | 0.10 | 0.09 | 0.09 | 0.00 | 0.09 | 0.06 | 0.00 | 0.00 | 0.00 |
| | IL-2 max | 0.40 | 0.64 | 1.02 | 0.52 | 0.33 | 0.81 | 0.24 | 0.48 | 2.17 |
| | TNFa median | 0.40 | 0.15 | 0.14 | 0.34 | 0.20 | 0.14 | 0.00 | 0.00 | 0.00 |
| | TNFa min | 0.10 | 0.09 | 0.00 | 0.06 | 0.00 | 0.00 | 0.00 | 0.00 | 0.00 |
| | TNFa max | 0.93 | 0.47 | 1.66 | 1.95 | 0.50 | 2.36 | 3.70 | 7.69 | 0.00 |
| IL-2/7/21 | IFNg median | 0.74 | 0.25 | 0.29 | 1.27 | 0.16 | 0.18 | 0.00 | 0.33 | 0.41 |
| | IFNg min | 0.14 | 0.00 | 0.11 | 0.07 | 0.00 | 0.00 | 0.00 | 0.00 | 0.00 |
| | IFNg max | 0.97 | 0.38 | 0.50 | 2.93 | 0.61 | 1.36 | 1.52 | 5.82 | 1.11 |
| | IL-2 median | 0.09 | 0.07 | 0.11 | 0.20 | 0.22 | 0.14 | 0.10 | 0.13 | 0.34 |
| | IL-2 min | 0.03 | 0.05 | 0.07 | 0.10 | 0.00 | 0.00 | 0.00 | 0.00 | 0.00 |
| | IL-2 max | 0.73 | 0.66 | 0.31 | 1.27 | 1.10 | 0.57 | 1.75 | 0.60 | 0.83 |
| | TNFa median | 0.19 | 0.19 | 0.20 | 0.45 | 0.33 | 0.59 | 0.39 | 0.53 | 0.10 |
| | TNFa min | 0.13 | 0.07 | 0.14 | 0.17 | 0.19 | 0.18 | 0.00 | 0.00 | 0.00 |
| | TNFa max | 1.70 | 0.31 | 0.26 | 2.55 | 1.01 | 2.58 | 0.94 | 0.72 | 0.93 |

Percent IFNy, IL-2 and TNFα production (median, minimum and maximum values) from the CD3+, CD4+ and CD8+ compartments of COVID-19 reactive T cells stimulated with the peptide libraries derived from S1 and S2 (N and C terminals of the S protein) after expansion under different cytokine stimulation conditions (IL-2/4/7, IL-2/7/15, IL-2/4/21 and IL-2/7/21).

TABLE 2

Cytokine production of COVID-19 reactive T cells from recovered donors expanded with different cytokine cocktails against S1 and S2 (N and C terminals of the S protein)

| | | CD3 % | | CD4 % | | CD8 % | |
|---|---|---|---|---|---|---|---|
| | | S1 | S2 | S1 | S2 | S1 | S2 |
| IL-2/4/7 | IFNg median | 5.81 | 3.88 | 6.40 | 1.55 | 3.02 | 3.26 |
| | IFNg min | 1.33 | 0.71 | 1.00 | 0.46 | 0.10 | 0.80 |
| | IFNg max | 26.50 | 8.54 | 20.80 | 6.60 | 13.20 | 6.29 |
| | IL2 median | 0.53 | 0.51 | 0.41 | 0.38 | 1.78 | 2.87 |
| | IL2 min | 0.14 | 0.24 | 0.06 | 0.07 | 0.00 | 0.15 |
| | IL2 max | 5.03 | 4.48 | 2.55 | 2.72 | 6.15 | 6.47 |
| | TNFa median | 3.24 | 1.41 | 2.56 | 0.87 | 5.51 | 1.80 |
| | TNFa min | 0.45 | 0.72 | 0.33 | 0.51 | 0.00 | 0.00 |
| | TNFa max | 9.47 | 2.63 | 12.80 | 1.60 | 27.30 | 14.70 |
| IL-2/7/15 | IFNg median | 5.94 | 2.99 | 2.61 | 2.27 | 3.59 | 2.22 |
| | IFNg min | 1.73 | 1.61 | 0.82 | 0.76 | 0.83 | 0.46 |
| | IFNg max | 18.00 | 14.60 | 15.80 | 10.50 | 10.10 | 7.77 |
| | IL2 median | 0.80 | 0.90 | 0.22 | 0.27 | 3.03 | 1.87 |
| | IL2 min | 0.12 | 0.05 | 0.05 | 0.03 | 0.14 | 0.40 |
| | IL2 max | 4.37 | 4.97 | 3.02 | 2.34 | 4.13 | 4.48 |
| | TNFa median | 2.20 | 2.02 | 2.62 | 1.36 | 10.27 | 11.38 |
| | TNFa min | 1.57 | 0.95 | 0.84 | 0.38 | 0.36 | 0.00 |
| | TNFa max | 10.50 | 4.14 | 13.90 | 3.05 | 26.20 | 22.60 |
| IL-2/4/21 | IFNg median | 0.17 | 0.14 | 0.14 | 0.22 | 0.00 | 0.00 |
| | IFNg min | 0.03 | 0.08 | 0.00 | 0.05 | 0.00 | 0.00 |
| | IFNg max | 4.15 | 0.26 | 6.21 | 0.60 | 0.67 | 0.00 |
| | IL-2 median | 0.17 | 0.16 | 0.14 | 0.16 | 0.00 | 0.00 |
| | IL-2 min | 0.00 | 0.08 | 0.00 | 0.00 | 0.00 | 0.00 |
| | IL-2 max | 0.40 | 0.57 | 0.68 | 0.44 | 0.66 | 0.61 |
| | TNFa median | 0.23 | 0.14 | 0.29 | 0.18 | 0.00 | 0.00 |
| | TNFa min | 0.07 | 0.05 | 0.08 | 0.00 | 0.00 | 0.00 |
| | TNFa max | 1.78 | 0.72 | 2.69 | 0.30 | 0.00 | 1.59 |
| IL-2/7/21 | IFNg median | 0.20 | 0.12 | 0.23 | 0.10 | 0.43 | 0.00 |
| | IFNg min | 0.05 | 0.05 | 0.00 | 0.00 | 0.00 | 0.00 |
| | IFNg max | 0.89 | 0.63 | 2.22 | 1.24 | 0.63 | 0.47 |
| | IL-2 median | 0.08 | 0.08 | 0.17 | 0.39 | 0.00 | 0.00 |
| | IL-2 min | 0.06 | 0.04 | 0.09 | 0.11 | 0.00 | 0.00 |
| | IL-2 max | 0.11 | 0.21 | 0.37 | 1.50 | 0.22 | 0.87 |
| | TNFa median | 0.20 | 0.09 | 0.35 | 0.21 | 0.00 | 0.00 |
| | TNFa min | 0.08 | 0.06 | 0.20 | 0.07 | 0.00 | 0.00 |
| | TNFa max | 0.37 | 0.31 | 1.18 | 1.50 | 0.63 | 0.83 |

Columns detail metal tags, antibody targets, clone numbers, and the vendor for antibodies used in mass cytometry analysis.

TABLE 3

Mass cytometry panel used for phenotyping of COVID-19 reactive T cells

| | | | |
|---|---|---|---|
| Tag | Antibody | Clone | Source |
| 89Y | CD45 | HI30 | Fluidigm |
| 141Pr | CC R6 | G034E3 | Fluidigm |
| 142Nd | IL4 | MP425D2 | Fluidigm |
| 143Nd | CD4 | OKT4 | Biolegend |
| 144Nd | IL2 | MQ1-17H12 | BD Biosciences |
| 145Nd | CD62L | DREG-56 | Biolegend |
| 146Nd | CD8 | RPA-T8 | Biolegend |
| 147Sm | CD127 | MB15-18C9 | Miltenyl Biotec |
| 148Nd | IL17a | BL168 | Biolegend |
| 149Sm | CD25 | 2A3 | BD Biosciences |
| 150Nd | CD28 | GD28.2 | BD Biosciences |
| 151Eu | CD107a | H4A3 | BD Biosciences |
| 1525m | CD95 | DX2 | BD Biosciences |
| 153Eu | CCR4 | 205410 | R&D Systems |
| 154Sm | TIGIT | MSA43 | Thermo Fisher |
| 155Gd | CD27 | M-T271 | BD Biosciences |
| 156Gd | CXCR4 | 12G5 | Biolegend |
| 188Gd | OX40 | ACT35 | Biolegend |
| 159Tb | Perforin | delta G9 | Miltenyl Biotec |
| 160Gd | CD45RA | Hl100 | Biolegend |
| 161Dy | TIM3 | REA635 | Miltenyl Biotec |
| 162Dy | Kl-67 | Kl-67 | Biolegend |
| 163Dy | CXCR3 | G025H7 | Biolegend |
| 164Dy | CD45RO | UCHL1 | Biolegend |
| 165Ho | CTLA-4 | L3D10 | Biolegend |
| 166Er | MIP1b | W15138A | Biolegend |
| 167Er | CCR7 | G043H7 | Biolegend |
| 168Er | IFNg | B27 | Biolegend |
| 169Tm | TNFa | MMAb1 1 | Biolegend |
| 170Er | HLA-D R | L243 | Biolegend |
| 171Yb | CD161 | 191B8 | Miltenyl |
| 172Yb | KLRG1 | 13F12F2 | Thermo Fisher |
| 173Yb | Granzyme B | GB11 | Fluidigm |
| 174Yb | PD1 | PD1.3.1.3 | Miltenyl Biotec |
| 175Lu | LAG3 | 11C3C65 | Biolegend |
| 176Yb | CD38 | REA572 | Miltenyl Biotec |
| 209Bi | 41BB | 4B4-1 | Biolegend |
| 115In | CD57 | HCD57 | Biolegend |
| Pt194 | CD3 | UCTH1 | Biolegend |
| Pt198 | LD | | |

Percent IFNy, IL-2 and TNFα production from total CD3+, and CD4+ and CD8+ subsets of COVID-19 reactive T cells derived from each recovered donor and stimulated with the peptide libraries derived from M, N and S structural proteins and fold expansion after culture with IL-2/4/7 or IL-2/7/15 cytokine cocktails.

TABLE 4

Cytokine production and fold expansion of COVID-19 reactive T cells derived from the different recovered donors and expanded with IL-2/4/7 or IL-2/7/15 cytokine cocktails

| | | | IL-2/4/7 | | | | | | | | |
|---|---|---|---|---|---|---|---|---|---|---|---|
| | | | CD3 % | | | CD4% | | | CD5% | | |
| | Fold Expansion | | M | N | S | M | N | S | M | N | S |
| Patient 1 | 177.37 | IFNg | 5.59 | 6.44 | 14.80 | 7.90 | 8.29 | 20.10 | 0.43 | 2.62 | 1.40 |
| | | 1L2 | 0.92 | 0.91 | 2.31 | 0.45 | 0.47 | 1.82 | 0.81 | 0.71 | 0.79 |
| | | TNFa | 3.18 | 2.67 | 6.52 | 3.14 | 3.10 | 7.12 | 1.45 | 1.13 | 2.03 |
| Patient 3 | 506.42 | IFNg | 1.22 | 117.10 | 13.50 | 0.91 | 18.90 | 15.00 | 3.77 | 0.81 | 1.25 |
| | | IL2 | 0.14 | 1.85 | 1.07 | 0.14 | 2.09 | 1.21 | 0.09 | 0.34 | 0.34 |
| | | TNFa | 0.67 | 7.50 | 3.99 | 0.57 | 8.41 | 4.59 | 0.66 | 0.00 | 0.11 |
| Patient 4 | 540.69 | IFNg | 2.74 | 10.20 | 3.84 | 2.51 | 9.01 | 3.24 | 0.21 | 5.08 | 0.32 |
| | | IL2 | 0.43 | 1.40 | 0.94 | 0.36 | 1.09 | 0.80 | 0.17 | 0.41 | 0.32 |
| | | TNFa | 1.66 | 8.35 | 3.23 | 1.63 | 5.06 | 3.26 | 1.05 | 3.01 | 2.42 |
| Patient 5 | 897.59 | IFNg | 2.94 | 2.87 | 10.90 | 2.70 | 2.41 | 10.50 | 1.06 | 19.40 | 0.61 |
| | | IL2 | 0.21 | 0.32 | 0.54 | 0.23 | 0.31 | 0.53 | 0.00 | 0.24 | 0.20 |
| | | TNFa | 1.23 | 1.18 | 4.42 | 1.30 | 1.28 | 4.64 | 0.53 | 3.07 | 0.61 |
| Patient 6 | 7.16 | IFNg | 0.11 | 0.12 | 0.21 | 0.14 | 0.14 | 0.22 | 0.10 | 0.05 | 0.25 |
| | | IL2 | 0.21 | 0.23 | 0.20 | 0.16 | 0.18 | 0.16 | 0.23 | 0.16 | 0.22 |
| | | TNFa | 0.19 | 0.22 | 0.25 | 0.05 | 0.04 | 0.07 | 0.10 | 0.12 | 0.32 |
| Patient 7 | 45572.50 | IFNg | 33.60 | 9.81 | 10.30 | 33.40 | 9.31 | 10.80 | 42.00 | 16.40 | 3.99 |
| | | IL2 | 1.41 | 1.10 | 0.97 | 1.44 | 1.11 | 0.99 | 0.61 | 0.43 | 0.22 |
| | | TNFa | 12.90 | 5.36 | 5.43 | 12.90 | 5.20 | 5.53 | 4.36 | 3.70 | 0.45 |
| Patient 8 | 2645.58 | IFNg | 6.81 | 3.33 | 4.87 | 5.77 | 3.46 | 5.48 | 25.90 | 4.53 | 0.27 |
| | | IL2 | 0.82 | 0.52 | 0.67 | 0.58 | 0.35 | 0.40 | 1.20 | 0.57 | 0.68 |
| | | TNFa | 2.80 | 0.87 | 1.71 | 2.64 | 0.88 | 1.81 | 5.65 | 0.97 | 0.68 |
| Patient 9 | 3537.23 | IFNg | 15.90 | 3.51 | 12.50 | 17.50 | 2.77 | 12.70 | 19.00 | 14.00 | 1.02 |
| | | IL2 | 3.80 | 1.16 | 1.34 | 3.51 | 0.93 | 1.32 | 1.13 | 1.12 | 0.18 |
| | | TNFa | 12.50 | 2.35 | 7.86 | 12.20 | 2.09 | 7.90 | 1.83 | 0.56 | 0.09 |

| | | | IL-2/7/15 | | | | | | | | |
|---|---|---|---|---|---|---|---|---|---|---|---|
| | | | CD3 % | | | CD4 % | | | CD8 % | | |
| | Fold Expansion | | M | N | S | M | N | S | M | N | S |
| Patient 1 | 183.62 | IFNg | 1.65 | 3.82 | 1.96 | 2.12 | 4.65 | 2.24 | 0.24 | 1.63 | 2.85 |
| | | IL2 | 1.08 | 1.56 | 1.64 | 0.49 | 0.81 | 0.92 | 0.38 | 0.23 | 1.75 |
| | | TNFa | 4.18 | 4.20 | 3.96 | 4.22 | 4.50 | 3.67 | 0.38 | 1.28 | 3.61 |
| Patient 3 | 141.91 | IFNg | 0.37 | 1.40 | 2.83 | 0.44 | 1.51 | 3.10 | 0.35 | 0.26 | 0.12 |
| | | IL2 | 0.15 | 0.47 | 0.45 | 0.20 | 0.53 | 0.51 | 1.27 | 0.90 | 1.18 |
| | | TNFa | 0.30 | 1.97 | 2.37 | 0.62 | 2.22 | 2.74 | 0.00 | 0.52 | 0.24 |
| Patient 4 | 1746.55 | IFNg | 8.67 | 8.12 | 26.00 | 8.70 | 4.21 | 29.49 | 0.27 | 20.50 | 0.77 |
| | | IL2 | 1.24 | 1.31 | 6.59 | 0.87 | 0.91 | 6.52 | 0.37 | 0.78 | 0.29 |
| | | TNFa | 3.93 | 3.60 | 10.10 | 3.99 | 2.41 | 11.20 | 0.69 | 7.27 | 0.78 |
| Patient 5 | 538.27 | IFNg | 3.43 | 3.28 | 6.08 | 3.29 | 3.00 | 5.64 | 0.30 | 2.41 | 1.53 |
| | | IL2 | 0.35 | 0.33 | 0.45 | 0.34 | 0.30 | 0.43 | 0.20 | 0.38 | 0.41 |
| | | TNFa | 1.40 | 1.40 | 2.31 | 1.52 | 1.46 | 2.47 | 0.91 | 1.25 | 0.32 |
| Patient 6 | 15.97 | IFNg | 0.15 | 0.17 | 0.48 | 0.13 | 0.21 | 0.42 | 0.09 | 0.04 | 0.34 |
| | | IL2 | 0.26 | 0.32 | 0.31 | 0.22 | 0.28 | 0.22 | 0.52 | 0.74 | 0.58 |
| | | TNFa | 0.24 | 0.48 | 0.78 | 0.10 | 0.60 | 0.33 | 0.09 | 0.10 | 0.25 |
| Patient 7 | 27716.81 | IFNg | 20.80 | 5.36 | 5.51 | 34.20 | 7.19 | 9.23 | 7.35 | 3.84 | 1.58 |
| | | IL2 | 0.98 | 0.59 | 0.65 | 1.64 | 0.85 | 0.95 | 0.26 | 0.26 | 0.27 |
| | | TNFa | 7.06 | 2.83 | 3.33 | 12.60 | 4.19 | 5.80 | 1.27 | 1.47 | 0.33 |
| Patient 8 | 2912.07 | IFNg | 19.10 | 15.60 | 3.43 | 17.30 | 16.10 | 3.46 | 47.30 | 5.02 | 0.93 |
| | | IL2 | 2.55 | 1.04 | 0.92 | 0.04 | 0.62 | 0.69 | 2.24 | 1.16 | 0.35 |
| | | TNFa | 4.90 | 1.70 | 1.91 | 3.90 | 1.43 | 1.82 | 18.50 | 1.45 | 1.14 |
| Patient 9 | 3088.38 | IFNg | 17.80 | 0.80 | 8.86 | 18.00 | 0.78 | 9.88 | 5.45 | 6.26 | 1.38 |
| | | IL2 | 2.12 | 0.40 | 0.93 | 2.08 | 0.36 | 0.91 | 0.33 | 0.49 | 0.45 |
| | | TNFa | 6.14 | 0.38 | 3.53 | 5.41 | 0.48 | 3.84 | 0.33 | 0.38 | 0.65 |

Percent IFNγy, IL-2 and TNFα production from total CD3+, and in CD4+ and CD8+ subsets of COVID-19 reactive T cells derived from each recovered donor and stimulated with the peptide libraries derived from M, N and S structural proteins and fold expansion after culture with IL-2/4/21 or IL-2/7/21 cytokine cocktails.

TABLE 5

Cytokine production and fold expansion of COVID-19 reactive T cells derived from the different recovered donors and expanded with IL-2/4/21 or IL-2/7/21 cytokine cocktails

| | | | IL-2/4/21 | | | | | | | | |
|---|---|---|---|---|---|---|---|---|---|---|---|
| | | | CD3% | | | CD4% | | | CD8 % | | |
| | Fold Expansion | | M | N | S | M | N | S | M | N | S |
| Patient 3 | 0.57 | IFNg | 0.03 | 0.14 | 0.09 | 0.00 | 0.09 | 0.16 | 0.00 | 0.00 | 0.00 |
| | | IL-2 | 0.12 | 0.09 | 0.09 | 0.13 | 0.09 | 0.16 | 0.00 | 0.00 | 0.00 |
| | | TNFa | 0.10 | 0.14 | 0.14 | 0.06 | 0.00 | 0.12 | 0.00 | 0.00 | 0.00 |
| Patient 4 | 0.53 | IFNg | 0.30 | 0.52 | 0.36 | 0.52 | 0.79 | 0.13 | 0.00 | 0.00 | 0.00 |
| | | IL-2 | 0.30 | 0.26 | 0.82 | 0.52 | 0.20 | 0.53 | 0.00 | 0.00 | 0.00 |
| | | TNFa | 0.40 | 0.17 | 0.21 | 0.26 | 0.00 | 0.13 | 0.00 | 7.88 | 0.00 |
| Patient 5 | 82.96 | IFNg | 1.11 | 0.20 | 2.53 | 1.81 | 0.29 | 3.63 | 0.00 | 0.09 | 0.00 |
| | | IL-2 | 0.12 | 0.13 | 0.13 | 0.13 | 0.09 | 0.15 | 0.00 | 0.00 | 2.17 |
| | | TNFa | 0.66 | 0.09 | 1.66 | 0.87 | 0.13 | 2.36 | 0.00 | 0.00 | 0.00 |
| Patient 6 | 0.06 | IFNg | 0.30 | 0.19 | 1.15 | 0.16 | 0.00 | 0.61 | 0.00 | 0.00 | 0.00 |
| | | IL-2 | 0.40 | 0.84 | 1.02 | 0.31 | 0.30 | 0.61 | 0.00 | 0.00 | 0.00 |
| | | TNFa | 0.90 | 0.47 | 0.00 | 0.47 | 0.45 | 0.00 | 3.70 | 0.00 | 0.00 |
| Patient 7 | 996.18 | IFNg | 2.46 | 0.64 | 0.55 | 4.99 | 1.08 | 1.00 | 3.37 | 0.22 | 0.63 |
| | | IL-2 | 0.10 | 0.10 | 0.15 | 0.11 | 0.10 | 0.06 | 0.24 | 0.00 | 0.00 |
| | | TNFa | 0.93 | 0.34 | 0.28 | 1.95 | 0.50 | 0.42 | 0.72 | 0.22 | 0.00 |
| Patient 8 | 0.71 | IFNg | 0.07 | 0.27 | 0.06 | 0.23 | 0.54 | 0.00 | 0.00 | 0.00 | 0.00 |
| | | IL-2 | 0.26 | 0.25 | 0.28 | 0.00 | 0.33 | 0.14 | 0.00 | 0.48 | 0.56 |
| | | TNFa | 0.19 | 0.13 | 0.12 | 0.23 | 0.33 | 0.14 | 0.00 | 0.00 | 0.00 |
| Patient 9 | 6.77 | IFNg | 0.45 | 0.15 | 0.12 | 0.51 | 0.24 | 0.11 | 1.48 | 0.00 | 0.97 |
| | | IL-2 | 0.16 | 0.21 | 0.16 | 0.16 | 0.24 | 0.16 | 0.00 | 0.00 | 0.00 |
| | | TNFa | 0.24 | 0.15 | 0.14 | 0.34 | 0.20 | 0.24 | 2.99 | 0.00 | 0.00 |

| | | | IL-2/7/21 | | | | | | | | |
|---|---|---|---|---|---|---|---|---|---|---|---|
| | | | CD3% | | | CD4% | | | CD6 % | | |
| | Fold Expansion | | M | N | S | M | N | S | M | N | S |
| Patient 3 | 0.85 | IFNg | 0.97 | 0.35 | 0.23 | 1.27 | 0.44 | 0.18 | 0.00 | 0.00 | 0.00 |
| | | IL-2 | 0.73 | 0.66 | 0.31 | 1.27 | 1.10 | 0.37 | 0.00 | 0.00 | 0.52 |
| | | TNFa | 1.70 | 0.31 | 0.17 | 2.55 | 0.88 | 0.18 | 0.00 | 0.72 | 0.00 |
| Patient 4 | 2.72 | IFNg | 0.21 | 0.38 | 0.29 | 0.07 | 0.16 | 0.09 | 0.00 | 0.33 | 0.72 |
| | | IL-2 | 0.09 | 0.05 | 0.09 | 0.17 | 0.00 | 0.09 | 0.40 | 0.33 | 0.00 |
| | | TNFa | 0.13 | 0.19 | 0.16 | 0.17 | 0.22 | 0.18 | 0.40 | 0.00 | 0.00 |
| Patient 5 | 109.68 | IFNg | 0.87 | 0.06 | 0.47 | 1.62 | 0.09 | 1.11 | 1.52 | 0.00 | 0.54 |
| | | IL-2 | 0.07 | 0.07 | 0.07 | 0.20 | 0.12 | 0.14 | 0.00 | 0.00 | 0.00 |
| | | TNFa | 0.18 | 0.07 | 0.20 | 0.44 | 0.20 | 0.59 | 0.76 | 0.57 | 0.54 |
| Patient 6 | 1.82 | IFNg | 0.21 | 0.17 | 0.13 | 0.18 | 0.00 | 0.00 | 0.23 | 0.21 | 0.34 |
| | | IL-2 | 0.16 | 0.05 | 0.11 | 0.45 | 0.51 | 0.00 | 0.10 | 0.07 | 0.34 |
| | | TNFa | 0.19 | 0.22 | 0.14 | 0.45 | 1.01 | 2.53 | 0.94 | 0.35 | 0.93 |
| Patient 7 | 415.98 | IFNg | 0.74 | 0.30 | 0.50 | 2.93 | 0.81 | 1.36 | 0.39 | 0.39 | 0.41 |
| | | IL-2 | 0.10 | 0.07 | 0.15 | 0.43 | 0.22 | 0.14 | 0.39 | 0.13 | 0.00 |
| | | TNFa | 0.44 | 0.18 | 0.25 | 2.22 | 0.81 | 0.61 | 0.39 | 0.26 | 0.10 |
| Patient 8 | 2.03 | IFNg | 0.14 | 0.21 | 0.11 | 0.23 | 0.09 | 0.11 | 0.00 | 5.52 | 0.00 |
| | | IL-2 | 0.06 | 0.05 | 0.11 | 0.10 | 0.12 | 0.20 | 1.75 | 0.53 | 0.33 |
| | | TNFa | 0.13 | 0.25 | 0.26 | 0.25 | 0.33 | 0.44 | 0.00 | 0.53 | 0.42 |
| Patient 9 | 76.48 | IFNg | 0.76 | 0.25 | 0.49 | 1.67 | 0.30 | 1.01 | 0.00 | 0.60 | 1.11 |
| | | IL-2 | 0.03 | 0.11 | 0.09 | 0.13 | 0.56 | 0.57 | 0.00 | 0.60 | 0.37 |
| | | TNFa | 0.19 | 0.14 | 0.25 | 0.52 | 0.19 | 0.64 | 0.00 | 0.60 | 0.00 |

Percent IFNγ, IL-2 and TNFα production from total CD3+, and CD4+ and CD8+ subsets of COVID-19 reactive T cells derived from each recovered donor and stimulated with the peptide libraries derived from S1 and S2 (N and C terminals of the S protein) after expansion with IL-2/4/7 or IL-2/7/15 cytokine cocktails.

TABLE 6

Cytokine production of COVID-19 reactive T cells from recovered donors expanded with IL-2/4/7 or IL-2/7/15 against S1 and S2 (N and C terminals of the S protein)

| | | IL-2/4/7 | | | | | |
|---|---|---|---|---|---|---|---|
| | | CD3 % | | CD4 % | | CD8 % | |
| | | S1 | S2 | S1 | S2 | S1 | S2 |
| Patient 1 | IFNg | 4.69 | 0.71 | 4.47 | 0.66 | 0.70 | 0.60 |
| | IL2 | 0.58 | 0.38 | 0.59 | 0.45 | 0.09 | 0.15 |
| | TNFa | 7.19 | 1.11 | 5.97 | 0.75 | 0.26 | 0.45 |
| Patient 3 | IFNg | 1.33 | 5.12 | 1.00 | 0.46 | 1.43 | 3.62 |
| | IL2 | 0.19 | 0.24 | 0.12 | 0.07 | 1.04 | 0.75 |
| | TNFa | 2.62 | 0.82 | 1.86 | 0.51 | 7.04 | 1.09 |
| Patient 4 | IFNg | 10.20 | 7.84 | 8.33 | 6.60 | 13.20 | 6.29 |
| | IL2 | 2.58 | 4.48 | 1.04 | 2.72 | 6.15 | 6.47 |
| | TNFa | 2.85 | 1.52 | 2.30 | 0.88 | 3.71 | 1.15 |
| Patient 5 | IFNg | 26.20 | 1.93 | 9.10 | 2.14 | 12.20 | 3.12 |
| | IL2 | 0.14 | 0.83 | 0.09 | 0.37 | 0.00 | 3.29 |
| | TNFa | 4.62 | 2.63 | 3.62 | 1.60 | 9.38 | 2.44 |
| Patient 6 | IFNg | 6.92 | 1.09 | 10.60 | 1.31 | 2.70 | 1.05 |
| | IL2 | 1.50 | 1.22 | 1.20 | 1.49 | 2.52 | 2.44 |
| | TNFa | 9.47 | 0.72 | 12.80 | 0.86 | 0.00 | 0.00 |
| Patient 7 | IFNg | 26.50 | 8.54 | 20.80 | 3.66 | 12.90 | 2.11 |
| | IL2 | 5.03 | 0.36 | 2.55 | 0.13 | 0.60 | 5.79 |
| | TNFa | 2.42 | 1.30 | 1.67 | 0.61 | 8.70 | 2.63 |
| Patient 8 | IFNg | 1.53 | 3.84 | 1.42 | 1.34 | 1.88 | 3.40 |
| | IL2 | 0.17 | 0.41 | 0.06 | 0.15 | 3.56 | 1.99 |
| | TNFa | 3.62 | 1.67 | 2.81 | 1.08 | 27.30 | 13.70 |
| Patient 9 | IFNg | 2.67 | 3.92 | 1.22 | 1.76 | 3.34 | 3.60 |
| | IL2 | 0.48 | 0.61 | 0.22 | 0.38 | 6.05 | 4.32 |
| | TNFa | 0.45 | 1.63 | 0.33 | 1.00 | 3.97 | 14.70 |

| | | IL-2/7/15 | | | | | |
|---|---|---|---|---|---|---|---|
| | | CD3% | | CD4% | | CD8% | |
| | | S1 | S2 | S1 | S2 | S1 | S2 |
| Patient 1 | IFNg | 5.96 | 3.25 | 4.47 | 3.72 | 0.83 | 7.77 |
| | IL2 | 0.8 | 0.58 | 0.59 | 0.69 | 4.13 | 3.88 |
| | TNFα | 7.59 | 1.17 | 5.97 | 0.93 | 5.79 | 0.97 |
| Patient 3 | IFNg | 7.43 | 1.61 | 1.05 | 0.76 | 4.57 | 1.72 |
| | IL2 | 0.41 | 0.13 | 0.07 | 0.04 | 3.28 | 0.54 |
| | TNFa | 5.06 | 2.48 | 3.70 | 1.45 | 26.20 | 18.40 |
| Patient 4 | IFNg | 18.00 | 14.60 | 2.50 | 1.32 | 10.10 | 5.90 |
| | IL2 | 1.57 | 1.29 | 0.22 | 0.12 | 1.04 | 0.75 |
| | TNFa | 1.94 | 1.93 | 1.53 | 1.31 | 8.33 | 8.86 |
| Patient 5 | IFNg | 4.99 | 1.96 | 0.82 | 1.81 | 5.86 | 0.46 |
| | IL2 | 0.12 | 0.05 | 0.05 | 0.03 | 3.15 | 0.46 |
| | TNFa | 1.63 | 4.14 | 0.84 | 3.05 | 12.20 | 22.60 |
| Patient 6 | IFNg | 3.95 | 2.72 | 1.97 | 2.53 | 1.22 | 1.20 |
| | IL2 | 0.80 | 0.24 | 0.21 | 0.09 | 1.28 | 0.40 |
| | TNFa | 2.46 | 2.29 | 1.51 | 1.41 | 18.70 | 15.30 |
| Patient 7 | IFNg | 11.10 | 1.72 | 15.80 | 2.00 | 2.70 | 1.05 |
| | IL2 | 4.37 | 3.80 | 3.02 | 2.34 | 3.60 | 3.48 |
| | TNFa | 10.50 | 0.95 | 13.90 | 1.03 | 0.36 | 0.00 |
| Patient 8 | IFNg | 1.73 | 4.34 | 2.71 | 10.50 | 1.81 | 3.70 |
| | IL2 | 4.97 | 0.12 | 0.18 | 1.13 | 0.14 | 4.48 |
| | TNFa | 1.57 | 2.10 | 1.38 | 2.25 | 16.10 | 13.90 |
| Patient 9 | IFNg | 5.92 | 5.03 | 7.23 | 6.43 | 4.48 | 2.71 |
| | IL2 | 1.20 | 1.21 | 0.47 | 0.42 | 2.90 | 2.98 |
| | TNFa | 1.59 | 1.41 | 4.48 | 0.38 | 0.40 | 0.27 |

Percent IFNγ, IL-2 and TNFα production from total CD3+, and CD4+ and CD8+ subsets of COVID-19 reactive T cells derived from each recovered donor and stimulated with the peptide libraries derived from S1 and S2 (N and C terminals of the S protein) after expansion with IL-2/4/21 or IL-2/7/21 cytokine cocktails.

TABLE 7

Cytokine production of COVID-19 reactive T cells from recovered donors expanded with IL-2/4/21 or IL-2/7/21 against S1 and S2 (N and C terminals of the S protein)

| | | IL-2/4/21 | | | | | |
|---|---|---|---|---|---|---|---|
| | | CD3% | | CD4% | | CD8% | |
| | | S1 | S2 | S1 | S2 | S1 | S2 |
| Patient 3 | IFNg | 0.04 | 0.08 | 0.02 | 0.05 | 0.00 | 0.00 |
| | IL-2 | 0.12 | 0.16 | 0.13 | 0.16 | 0.00 | 0.00 |
| | TNFa | 0.08 | 0.05 | 0.08 | 0.04 | 0.00 | 1.59 |
| Patient 4 | IFNg | 0.17 | 0.19 | 0.14 | 0.23 | 0.00 | 0.00 |
| | IL-2 | 0.40 | 0.57 | 0.08 | 0.23 | 0.00 | 0.00 |
| | TNFa | 0.40 | 0.19 | 0.27 | 0.00 | 0.00 | 0.00 |
| Patient 5 | IFNg | 4.15 | 0.14 | 6.21 | 0.22 | 0.00 | 0.00 |
| | IL-2 | 0.17 | 0.08 | 0.24 | 0.09 | 0.00 | 0.00 |
| | TNFa | 1.78 | 0.13 | 2.69 | 0.22 | 0.00 | 0.00 |
| Patient 6 | IFNg | 0.33 | 0.14 | 0.17 | 0.22 | 0.00 | 0.00 |
| | IL-2 | 0.00 | 0.57 | 0.00 | 0.44 | 0.00 | 0.00 |
| | TNFa | 1.00 | 0.72 | 0.34 | 0.22 | 0.00 | 0.00 |
| Patient 7 | IFNg | 0.88 | 0.21 | 1.12 | 0.60 | 0.67 | 0.00 |
| | IL-2 | 0.21 | 0.18 | 0.31 | 0.22 | 0.22 | 0.33 |
| | TNFa | 0.23 | 0.12 | 0.35 | 0.16 | 0.00 | 0.67 |
| Patient 8 | IFNg | 0.08 | 0.09 | 0.00 | 0.14 | 0.00 | 0.00 |
| | IL-2 | 0.35 | 0.16 | 0.14 | 0.00 | 0.66 | 0.61 |
| | TNFa | 0.18 | 0.14 | 0.29 | 0.00 | 0.00 | 0.00 |
| Patient 9 | IFNg | 0.03 | 0.26 | 0.08 | 0.50 | 0.00 | 0.00 |
| | IL-2 | 0.10 | 0.13 | 0.11 | 0.12 | 0.00 | 0.00 |
| | TNFa | 0.07 | 0.20 | 0.16 | 0.30 | 0.00 | 0.00 |

| | | IL-2/7/21 | | | | | |
|---|---|---|---|---|---|---|---|
| | | CD3% | | CD4% | | CD8% | |
| | | S1 | S2 | S1 | S2 | S1 | S2 |
| Patient 3 | IFNg | 0.05 | 0.10 | 0.00 | 0.00 | 0.58 | 0.00 |
| | IL-2 | 0.11 | 0.21 | 0.09 | 0.43 | 0.00 | 0.69 |
| | TNFa | 0.22 | 0.07 | 0.35 | 0.14 | 0.00 | 0.00 |
| Patient 4 | IFNg | 0.20 | 0.13 | 0.23 | 0.10 | 0.00 | 0.00 |
| | IL-2 | 0.07 | 0.21 | 0.10 | 0.24 | 0.00 | 0.87 |
| | TNFa | 0.18 | 0.15 | 0.20 | 0.07 | 0.00 | 0.00 |
| Patient 5 | IFNg | 0.89 | 0.05 | 2.22 | 0.08 | 0.00 | 0.00 |
| | IL-2 | 0.08 | 0.14 | 0.17 | 0.11 | 0.00 | 0.83 |
| | TNFa | 0.37 | 0.06 | 1.01 | 0.15 | 0.00 | 0.83 |
| Patient 6 | IFNg | 0.20 | 0.13 | 0.34 | 1.00 | 0.43 | 0.10 |
| | IL-2 | 0.08 | 0.08 | 0.34 | 1.50 | 0.22 | 0.00 |
| | TNFa | 0.18 | 0.07 | 1.02 | 1.50 | 0.43 | 0.50 |
| Patient 7 | IFNg | 0.31 | 0.12 | 0.88 | 0.21 | 0.63 | 0.47 |
| | IL-2 | 0.05 | 0.04 | 0.11 | 0.39 | 0.00 | 0.00 |
| | TNFa | 0.20 | 0.09 | 1.18 | 0.21 | 0.63 | 0.00 |
| Patient 8 | IFNg | 0.10 | 0.09 | 0.13 | 0.05 | 0.56 | 0.00 |
| | IL-2 | 0.08 | 0.07 | 0.16 | 0.18 | 0.00 | 0.00 |
| | TNFa | 0.20 | 0.23 | 0.31 | 0.55 | 0.00 | 0.00 |
| Pattent 9 | IFNg | 0.05 | 0.63 | 0.04 | 1.24 | 0.27 | 0.35 |
| | IL-2 | 0.08 | 0.07 | 0.37 | 0.41 | 0.00 | 0.00 |
| | TNFa | 0.08 | 0.31 | 0.20 | 0.85 | 0.54 | 0.70 |

Percent IFNγ, IL-2 and TNFα production (median, minimum and maximum values) from total CD3+, and CD4+ and CD8+ subsets of COVID-19 reactive T cells stimulated with the peptide libraries derived from M, N and S structural proteins after expansion with the two different cytokine cocktails IL-2/4/7 or IL-2/7/15.

TABLE 8

Cytokine production of COVID-19 reactive T cells from healthy controls expanded with different cytokine cocktails against M, N and S structural proteins

| | | CD3% | | | CD4% | | | CD8% | | |
|---|---|---|---|---|---|---|---|---|---|---|
| | | M | N | S | M | N | S | M | N | S |
| IL-2/4/7 | IFNg median | 0.43 | 0.29 | 0.44 | 0.55 | 0.23 | 0.34 | 0.11 | 0.18 | 0.24 |
| | IFNg min | 0.13 | 0.16 | 0.19 | 0.14 | 0.16 | 0.06 | 0.02 | 0.07 | 0.04 |
| | IFNg max | 1.37 | 0.67 | 2.57 | 2.59 | 1.12 | 2.50 | 0.19 | 0.67 | 2.59 |
| | IL-2 median | 0.14 | 0.50 | 0.16 | 0.10 | 0.18 | 0.10 | 0.24 | 0.18 | 0.24 |
| | IL-2 min | 0.03 | 0.12 | 0.04 | 0.03 | 0.14 | 0.04 | 0.00 | 0.09 | 0.02 |
| | IL-2 max | 0.31 | 0.74 | 0.63 | 0.23 | 1.27 | 0.17 | 0.41 | 0.63 | 0.29 |
| | TNFa median | 0.25 | 0.23 | 0.16 | 0.26 | 0.57 | 0.27 | 0.04 | 0.22 | 0.19 |
| | TNFa min | 0.15 | 0.17 | 0.06 | 0.11 | 0.07 | 0.07 | 0.02 | 0.12 | 0.02 |
| | TNFa max | 1.11 | 2.34 | 1.59 | 2.19 | 5.17 | 3.55 | 0.18 | 0.28 | 0.67 |
| IL-2/7/15 | IFNg median | 0.24 | 0.15 | 0.59 | 0.27 | 0.18 | 0.37 | 0.10 | 0.13 | 0.11 |
| | IFNg min | 0.10 | 0.09 | 0.21 | 0.17 | 0.05 | 0.16 | 0.07 | 0.08 | 0.06 |
| | IFNg max | 2.13 | 0.39 | 1.64 | 6.63 | 0.49 | 1.58 | 0.91 | 0.24 | 1.22 |
| | IL-2 median | 0.17 | 0.12 | 0.18 | 0.10 | 0.12 | 0.11 | 0.11 | 0.08 | 0.15 |
| | IL-2 min | 0.05 | 0.00 | 0.07 | 0.09 | 0.00 | 0.07 | 0.02 | 0.00 | 0.07 |
| | IL-2 max | 0.25 | 0.25 | 0.29 | 0.25 | 0.14 | 0.31 | 0.22 | 0.21 | 0.27 |
| | TNFa median | 0.12 | 0.12 | 0.18 | 0.24 | 0.21 | 0.31 | 0.10 | 0.10 | 0.14 |
| | TNFa min | 0.09 | 0.02 | 0.09 | 0.10 | 0.00 | 0.14 | 0.06 | 0.01 | 0.08 |
| | TNFa max | 1.44 | 0.14 | 0.38 | 4.88 | 0.23 | 1.14 | 0.24 | 0.18 | 0.22 |

Percent IFNγ, IL-2 and TNFα production from total CD3+, and CD4+ and CD8+ subsets of COVID-19 reactive T cells derived from each healthy donor and stimulated with the peptide libraries derived from M, N and S structural proteins and fold expansion after culture with IL-2/4/7 or IL-2/7/15 cytokine cocktails.

TABLE 9

Cytokine production and fold expansion of COVID-19 reactive T cells derived from the different healthy controls and expanded with IL-2/4/7 or IL-2/7/15 cytokine cocktails

| | | | lL-2/4/7 | | | | | | | | |
|---|---|---|---|---|---|---|---|---|---|---|---|
| | | | CD3% | | | CD4% | | | CD8% | | |
| | Fold Expansion | | M | N | S | M | N | S | M | N | S |
| HC 1 | 21.31 | IFNg | 0.14 | 0.29 | 2.57 | 0.17 | 0.32 | 2.16 | 0.02 | 0.07 | 0.04 |
| | | IL2 | 0.31 | 0.74 | 0.83 | 0.11 | 0.14 | 0.07 | 0.25 | 0.63 | 0.29 |
| | | TNFα | 0.15 | 0.23 | 0.06 | 0.14 | 0.27 | 0.58 | 0.02 | 0.22 | 0.11 |
| HC 2 | 8.13 | IFNg | 0.13 | 0.25 | 0.44 | 0.14 | 0.28 | 0.24 | 0.19 | 0.30 | 0.22 |
| | | IL2 | 0.25 | 0.50 | 0.27 | 0.10 | 0.16 | 0.10 | 0.41 | 0.30 | 0.21 |
| | | TNFα | 0.16 | 0.21 | 0.16 | 0.26 | 0.57 | 0.27 | 0.18 | 0.27 | 0.19 |
| HC 3 | 2.85 | IFNg | 0.43 | 0.18 | 0.19 | 0.55 | 0.16 | 0.06 | 0.11 | 0.18 | 0.24 |
| | | IL2 | 0.14 | 0.12 | 0.13 | 0.23 | 0.15 | 0.10 | 0.24 | 0.18 | 0.05 |
| | | TNFα | 0.25 | 0.17 | 0.12 | 0.11 | 0.07 | 0.07 | 0.17 | 0.12 | 0.38 |
| HC4 | 27.74 | IFNg | 1.37 | 0.67 | 0.33 | 1.57 | 1.12 | 0.34 | 0.08 | 0.17 | 0.41 |
| | | IL2 | 0.04 | 0.65 | 0.04 | 0.04 | 1.27 | 0.04 | 0.00 | 0.15 | 0.02 |
| | | TNFα | 1.11 | 2.34 | 0.22 | 1.36 | 5.17 | 0.27 | 0.03 | 0.28 | 0.02 |
| HC5 | 41.84 | IFNg | 0.84 | 0.60 | 1.83 | 2.59 | 0.27 | 2.50 | 0.12 | 0.67 | 2.59 |
| | | IL2 | 0.03 | 0.15 | 0.15 | 0.03 | 0.22 | 0.17 | 0.03 | 0.09 | 0.24 |
| | | TNFα | 0.63 | 0.62 | 1.59 | 2.19 | 0.96 | 3.55 | 0.04 | 0.22 | 0.57 |

| | | | IL-2/7/15 | | | | | | | | |
|---|---|---|---|---|---|---|---|---|---|---|---|
| | | | CD3% | | | CD4% | | | CD8% | | |
| | Fold Expansion | | M | N | S | M | N | S | M | N | S |
| HC1 | 15.73 | IFNg | 0.24 | 0.23 | 0.56 | 0.26 | 0.30 | 0.36 | 0.10 | 0.10 | 0.11 |
| | | IL-2 | 0.25 | 0.25 | 0.29 | 0.09 | 0.12 | 0.09 | 0.22 | 0.18 | 0.18 |
| | | TNFa | 0.08 | 0.11 | 0.09 | 0.13 | 0.21 | 0.23 | 0.07 | 0.10 | 0.96 |
| HC2 | 9.01 | IFNg | 0.10 | 0.11 | 0.21 | 0.17 | 0.13 | 0.16 | 0.07 | 0.17 | 0.08 |
| | | IL-2 | 0.17 | 0.13 | 0.18 | 0.10 | 0.12 | 0.11 | 0.15 | 0.21 | 0.27 |
| | | TNFa | 0.12 | 0.14 | 0.15 | 0.14 | 0.22 | 0.34 | 0.10 | 0.13 | 0.11 |
| HC 3 | 4.00 | IFNg | 0.24 | 0.15 | 0.59 | 0.27 | 0.18 | 0.37 | 0.10 | 0.08 | 0.09 |
| | | IL-2 | 0.13 | 0.12 | 0.14 | 0.13 | 0.14 | 0.18 | 0.10 | 0.08 | 0.13 |
| | | TNFa | 0.12 | 0.13 | 0.18 | 0.10 | 0.11 | 0.14 | 0.22 | 0.18 | 0.15 |
| HC4 | 53.95 | IFNg | 2.13 | 0.09 | 0.67 | 6.63 | 0.05 | 0.40 | 0.21 | 0.24 | 0.96 |
| | | IL-2 | 0.05 | 0.00 | 0.07 | 0.10 | 0.00 | 0.07 | 0.02 | 0.00 | 0.07 |
| | | TNFa | 1.44 | 0.02 | 0.33 | 4.66 | 0.00 | 0.31 | 0.06 | 0.07 | 0.22 |
| HC5 | 24.77 | IFNg | 1.23 | 0.39 | 1.64 | 1.24 | 0.49 | 1.58 | 0.91 | 0.13 | 1.22 |
| | | IL-2 | 0.17 | 0.00 | 0.18 | 0.25 | 0.01 | 0.31 | 0.11 | 0.01 | 0.15 |
| | | TNFa | 0.47 | 0.07 | 0.38 | 1.06 | 0.23 | 1.14 | 0.24 | 0.01 | 0.14 |

### SEQUENCES

SEQ ID NO:1

1 attaaaggtt tataccttcc caggtaacaa accaaccaac tttcgatctc ttgtagatct
61 gttctctaaa cgaactttaa aatctgtgtg gctgtcactc ggctgcatgc ttagtgcact
121 cacgcagtat aattaataac taattactgt cgttgacagg acacgagtaa ctcgtctatc
181 ttctgcaggc tgcttacggt ttcgtccgtg ttgcagccga tcatcagcac atctaggttt
241 cgtccgggtg tgaccgaaag gtaagatgga gagccttgtc cctggtttca acgagaaaac
301 acacgtccaa ctcagtttgc ctgttttaca ggttcgcgac gtgctcgtac gtggctttgg
361 agactccgtg gaggaggtct tatcagaggc acgtcaacat cttaaagatg gcacttgtgg
421 cttagtagaa gttgaaaaag gcgttttgcc tcaacttgaa cagccctatg tgttcatcaa
481 acgttcggat gctcgaactg cacctcatgg tcatgttatg gttgagctgg tagcagaact
541 cgaaggcatt cagtacggtc gtagtggtga gacacttggt gtccttgtcc ctcatgtggg
601 cgaaatacca gtggcttacc gcaaggttct tcttcgtaag aacggtaata aaggagctgg
661 tggccatagt tacggcgccg atctaaagtc atttgactta ggcgacgagc ttggcactga
721 tccttatgaa gattttcaag aaaactggaa cactaaacat agcagtggtg ttacccgtga
781 actcatgcgt gagcttaacg gaggggcata cactcgctat gtcgataaca acttctgtgg
841 ccctgatggc taccctcttg agtgcattaa agaccttcta gcacgtgctg gtaaagcttc
901 atgcactttg tccgaacaac tggactttat tgacactaag aggggtgtat actgctgccg
961 tgaacatgag catgaaattg cttggtacac ggaacgttct gaaaagagct atgaattgca
1021 gacacctttt gaaattaaat tggcaaagaa atttgacacc ttcaatgggg aatgtccaaa
1081 ttttgtattt cccttaaatt ccataatcaa gactattcaa ccaagggttg aaaagaaaaa
1141 gcttgatggc tttatgggta gaattcgatc tgtctatcca gttgcgtcac caaatgaatg
1201 caaccaaatg tgcctttcaa ctctcatgaa gtgtgatcat tgtggtgaaa cttcatggca
1261 gacgggcgat tttgttaaag ccacttgcga attttgtggc actgagaatt tgactaaaga
1321 aggtgccact acttgtggtt acttacccca aaatgctgtt gttaaaattt attgtccagc
1381 atgtcacaat tcagaagtag gacctgagca tagtcttgcc gaataccata atgaatctgg
1441 cttgaaaacc attcttcgta agggtggtcg cactattgcc tttggaggct gtgtgttctc
1501 ttatgttggt tgccataaca agtgtgccta ttgggttcca cgtgctagcg ctaacatagg
1561 ttgtaaccat acaggtgttg ttggagaagg ttccgaaggt cttaatgaca accttcttga
1621 aatactccaa aaagagaaag tcaacatcaa tattgttggt gactttaaac ttaatgaaga
1681 gatcgccatt attttggcat ctttttctgc ttccacaagt gcttttgtgg aaactgtgaa
1741 aggtttggat tataaagcat tcaaacaaat tgttgaatcc tgtggtaatt ttaaagttac
1801 aaaaggaaaa gctaaaaaag gtgcctggaa tattggtgaa cagaaatcaa tactgagtcc
1861 tctttatgca tttgcatcag aggctgctcg tgttgtacga tcaattttct cccgcactct
1921 tgaaactgct caaaattctg tgcgtgtttt acagaaggcc gctataacaa tactagatgg
1981 aatttcacag tattcactga gactcattga tgctatgatg ttcacatctg atttggctac
2041 taacaatcta gttgtaatgg cctacattac aggtggtgtt gttcagttga cttcgcagtg
2101 gctaactaac atctttggca ctgtttatga aaaactcaaa cccgtccttg attggcttga
2161 agagaagttt aaggaaggtg tagagtttct tagagacggt tgggaaattg ttaaatttat
2221 ctcaacctgt gcttgtgaaa ttgtcggtgg acaaattgtc acctgtgcaa aggaaattaa
2281 ggagagtgtt cagacattct ttaagcttgt aaataaattt ttggctttgt gtgctgactc
2341 tatcattatt ggtggagcta aacttaaagc cttgaattta ggtgaaacat ttgtcacgca
2401 ctcaaaggga ttgtacagaa agtgtgttaa atccagagaa gaaactggcc tactcatgcc
2461 tctaaaagcc ccaaaagaaa ttatcttctt agagggagaa acacttccca cagaagtgtt
2521 aacagaggaa gttgtcttga aaactggtga tttacaacca ttagaacaac ctactagtga
2581 agctgttgaa gctccattgg ttggtacacc agtttgtatt aacgggctta tgttgctcga
2641 aatcaaagac acagaaaagt actgtgccct tgcacctaat atgatggtaa caaacaatac
2701 cttcacactc aaaggcggtg caccaacaaa ggttactttt ggtgatgaca ctgtgataga
2761 agtgcaaggt tacaagagtg tgaatatcac ttttgaactt gatgaaagga ttgataaagt
2821 acttaatgag aagtgctctg cctatacagt tgaactcggt acagaagtaa atgagttcgc
2881 ctgtgttgtg gcagatgctg tcataaaaac tttgcaacca gtatctgaat tacttacacc
2941 actgggcatt gatttagatg agtggagtat ggctacatac tacttatttg atgagtctgg
3001 tgagtttaaa ttggcttcac atatgtattg ttctttctac cctccagatg aggatgaaga
3061 agaaggtgat tgtgaagaag aagagtttga gccatcaact caatatgagt atggtactga
3121 agatgattac caaggtaaac ctttggaatt tggtgccact tctgctgctc ttcaacctga
3181 agaagagcaa gaagaagatt ggttagatga tgatagtcaa caaactgttg gtcaacaaga
3241 cggcagtgag gacaatcaga caactactat tcaaacaatt gttgaggttc aacctcaatt
3301 agagatggaa cttacaccag ttgttcagac tattgaagtg aatagtttta gtggttattt
3361 aaaacttact gacaatgtat acattaaaaa tgcagacatt gtggaagaag ctaaaaaggt
3421 aaaaccaaca gtggttgtta atgcagccaa tgtttacctt aaacatggag gaggtgttgc
3481 aggagcctta aataaggcta ctaacaatgc catgcaagtt gaatctgatg attacatagc
3541 tactaatgga ccacttaaag tgggtggtag ttgtgtttta agcggacaca atcttgctaa
3601 acactgtctt catgttgtcg gcccaaatgt taacaaaggt gaagacattc aacttcttaa
3661 gagtgcttat gaaaatttta atcagcacga agttctactt gcaccattat tatcagctgg
3721 tatttttggt gctgacccta tacattcttt aagagtttgt gtagatactg ttcgcacaaa
3781 tgtctactta gctgtctttg ataaaaatct ctatgacaaa cttgtttcaa gctttttgga
3841 aatgaagagt gaaaagcaag ttgaacaaaa gatcgctgag attcctaaag aggaagttaa
3901 gccatttata actgaaagta aaccttcagt tgaacagaga aaacaagatg ataagaaaat
3961 caaagcttgt gttgaagaag ttacaacaac tctggaagaa actaagttcc tcacagaaaa
4021 cttgttactt tatattgaca ttaatggcaa tcttcatcca gattctgcca ctcttgttag
4081 tgacattgac atcactttct taaagaaaga tgctccatat atagtgggtg atgttgttca
4141 agagggtgtt ttaactgctg tggttatacc tactaaaaag gctggtggca ctactgaaat
4201 gctagcgaaa gctttgagaa aagtgccaac agacaattat ataaccactt acccgggtca
4261 gggtttaaat ggttacactg tagaggaggc aaagacagtg cttaaaaagt gtaaaagtgc
4321 cttttacatt ctaccatcta ttatctctaa tgagaagcaa gaaattcttg gaactgtttc
4381 ttggaatttg cgagaaatgc ttgcacatgc agaagaaaca cgcaaattaa tgcctgtctg
4441 tgtggaaact aaagccatag tttcaactat acagcgtaaa tataagggta ttaaaataca
4501 agagggtgtg gttgattatg gtgctagatt ttacttttac accagtaaaa caactgtagc
4561 gtcacttatc aacacactta acgatctaaa tgaaactctt gttacaatgc cacttggcta
4621 tgtaacacat ggcttaaatt tggaagaagc tgctcggtat atgagatctc tcaaagtgcc
4681 agctacagtt tctgtttctt cacctgatgc tgttacagcg tataatggtt atcttacttc
4741 ttcttctaaa acacctgaag aacattttat tgaaaccatc tcacttgctg gttcctataa
4801 agattggtcc tattctggac aatctacaca actaggtata gaatttctta agagaggtga
4861 taaaagtgta tattacacta gtaatcctac cacattccac ctagatggtg aagttatcac
4921 ctttgacaat cttaagacac ttctttcttt gagagaagtg aggactatta aggtgtttac
4981 aacagtagac aacattaacc tccacacgca agttgtggac atgtcaatga catatggaca
5041 acagtttggt ccaacttatt tggatggagc tgatgttact aaaataaaac ctcataattc
5101 acatgaaggt aaaacatttt atgttttacc taatgatgac actctacgtg ttgaggcttt
5161 tgagtactac cacacaactg atcctagttt tctgggtagg tacatgtcag cattaaatca
5221 cactaaaaag tggaaatacc cacaagttaa tggtttaact tctattaaat gggcagataa
5281 caactgttat cttgccactg cattgttaac actccaacaa atagagttga agtttaatcc
5341 acctgctcta caagatgctt attacagagc aagggctggt gaagctgcta acttttgtgc
5401 acttatctta gcctactgta ataagacagt aggtgagtta ggtgatgtta gagaaacaat
5461 gagttacttg tttcaacatg ccaatttaga ttcttgcaaa agagtcttga acgtggtgtg
5521 taaaacttgt ggacaacagc agacaaccct taagggtgta gaagctgtta tgtacatggg
5581 cacactttct tatgaacaat ttaagaaagg tgttcagata ccttgtacgt gtggtaaaca
5641 agctacaaaa tatctagtac aacaggagtc accttttgtt atgatgtcag caccacctgc
5701 tcagtatgaa cttaagcatg gtacatttac ttgtgctagt gagtacactg gtaattacca
5761 gtgtggtcac tataaacata taacttctaa agaaactttg tattgcatag acggtgcttt
5821 acttacaaag tcctcagaat acaaaggtcc tattacggat gttttctaca aagaaaacag
5881 ttacacaaca accataaaac cagttactta taaattggat ggtgttgttt gtacagaaat
5941 tgaccctaag ttggacaatt attataagaa agacaattct tatttcacag agcaaccaat
6001 tgatcttgta ccaaaccaac catatccaaa cgcaagcttc gataatttta agtttgtatg
6061 tgataatatc aaatttgctg atgatttaaa ccagttaact ggttataaga aacctgcttc
6121 aagagagctt aaagttacat ttttccctga cttaaatggt gatgtggtgg ctattgatta
6181 taaacactac acaccctctt ttaagaaagg agctaaattg ttacataaac ctattgtttg
6241 gcatgttaac aatgcaacta ataaagccac gtataaacca aatacctggt gtatacgttg
6301 tctttggagc acaaaaccag ttgaaacatc aaattcgttt gatgtactga agtcagagga
6361 cgcgcaggga atggataatc ttgcctgcga agatctaaaa ccagtctctg aagaagtagt
6421 ggaaaatcct accatacaga aagacgttct tgagtgtaat gtgaaaacta ccgaagttgt
6481 aggagacatt atacttaaac cagcaaataa tagtttaaaa attacagaag aggttggcca
6541 cacagatcta atggctgctt atgtagacaa ttctagtctt actattaaga aacctaatga
6601 attatctaga gtattaggtt tgaaaaccct tgctactcat ggtttagctg ctgttaatag
6661 tgtcccttgg gatactatag ctaattatgc taagcctttt cttaacaaag ttgttagtac
6721 aactactaac atagttacac ggtgtttaaa ccgtgtttgt actaattata tgccttattt
6781 ctttacttta ttgctacaat tgtgtacttt tactagaagt acaaattcta gaattaaagc
6841 atctatgccg actactatag caaagaatac tgttaagagt gtcggtaaat tttgtctaga
6901 ggcttcattt aattatttga agtcacctaa tttttctaaa ctgataaata ttataatttg
6961 gtttttacta ttaagtgttt gcctaggttc tttaatctac tcaaccgctg ctttaggtgt
7021 tttaatgtct aatttaggca tgccttctta ctgtactggt tacagagaag gctatttgaa
7081 ctctactaat gtcactattg caacctactg tactggttct ataccttgta gtgtttgtct
7141 tagtggttta gattctttag acacctatcc ttctttagaa actatacaaa ttaccatttc
7201 atcttttaaa tgggatttaa ctgcttttgg cttagttgca gagtggtttt tggcatatat
7261 tcttttcact aggtttttct atgtacttgg attggctgca atcatgcaat tgtttttcag
7321 ctattttgca gtacatttta ttagtaattc ttggcttatg tggttaataa ttaatcttgt
7381 acaaatggcc ccgatttcag ctatggttag aatgtacatc ttctttgcat cattttatta
7441 tgtatggaaa agttatgtgc atgttgtaga cggttgtaat tcatcaactt gtatgatgtg
7501 ttacaaacgt aatagagcaa caagagtcga atgtacaact attgttaatg gtgttagaag
7561 gtccttttat gtctatgcta atggaggtaa aggcttttgc aaactacaca attggaattg
7621 tgttaattgt gatacattct gtgctggtag tacatttatt agtgatgaag ttgcgagaga
7681 cttgtcacta cagtttaaaa gaccaataaa tcctactgac cagtcttctt acatcgttga
7741 tagtgttaca gtgaagaatg gttccatcca tctttacttt gataaagctg gtcaaaagac
7801 ttatgaaaga cattctctct ctcattttgt taacttagac aacctgagag ctaataacac
7861 taaaggttca ttgcctatta atgttatagt ttttgatggt aaatcaaaat gtgaagaatc
7921 atctgcaaaa tcagcgtctg tttactacag tcagcttatg tgtcaaccta tactgttact
7981 agatcaggca ttagtgtctg atgttggtga tagtgcggaa gttgcagtta aaatgtttga
8041 tgcttacgtt aatacgtttt catcaacttt taacgtacca atggaaaaac tcaaaacact
8101 agttgcaact gcagaagctg aacttgcaaa gaatgtgtcc ttagacaatg tcttatctac
8161 ttttatttca gcagctcggc aagggtttgt tgattcagat gtagaaacta aagatgttgt
8221 tgaatgtctt aaattgtcac atcaatctga catagaagtt actggcgata gttgtaataa
8281 ctatatgctc acctataaca aagttgaaaa catgacaccc cgtgaccttg gtgcttgtat
8341 tgactgtagt gcgcgtcata ttaatgcgca ggtagcaaaa agtcacaaca ttgctttgat
8401 atggaacgtt aaagatttca tgtcattgtc tgaacaacta cgaaaacaaa tacgtagtgc
8461 tgctaaaaag aataacttac cttttaagtt gacatgtgca actactagac aagttgttaa
8521 tgttgtaaca acaaagatag cacttaaggg tggtaaaatt gttaataatt ggttgaagca
8581 gttaattaaa gttacacttg tgttcctttt tgttgctgct attttctatt taataacacc
8641 tgttcatgtc atgtctaaac atactgactt ttcaagtgaa atcataggat acaaggctat
8701 tgatggtggt gtcactcgtg acatagcatc tacagatact tgttttgcta acaaacatgc
8761 tgattttgac acatggttta gccagcgtgg tggtagttat actaatgaca aagcttgccc
8821 attgattgct gcagtcataa caagagaagt gggttttgtc gtgcctggtt tgcctggcac
8881 gatattacgc acaactaatg gtgacttttt gcatttctta cctagagttt ttagtgcagt
8941 tggtaacatc tgttacacac catcaaaact tatagagtac actgactttg caacatcagc
9001 ttgtgttttg gctgctgaat gtacaatttt taaagatgct tctggtaagc cagtaccata
9061 ttgttatgat accaatgtac tagaaggttc tgttgcttat gaaagtttac gccctgacac
9121 acgttatgtg ctcatggatg gctctattat tcaatttcct aacacctacc ttgaaggttc
9181 tgttagagtg gtaacaactt ttgattctga gtactgtagg cacggcactt gtgaaagatc
9241 agaagctggt gtttgtgtat ctactagtgg tagatgggta cttaacaatg attattacag
9301 atctttacca ggagttttct gtggtgtaga tgctgtaaat ttacttacta atatgtttac
9361 accactaatt caacctattg gtgctttgga catatcagca tctatagtag ctggtggtat
9421 tgtagctatc gtagtaacat gccttgccta ctattttatg aggtttagaa gagcttttgg
9481 tgaatacagt catgtagttg cctttaatac tttactattc cttatgtcat tcactgtact
9541 ctgtttaaca ccagtttact cattcttacc tggtgtttat tctgttattt acttgtactt
9601 gacattttat cttactaatg atgtttcttt tttagcacat attcagtgga tggttatgtt
9661 cacaccttta gtacctttct ggataacaat tgcttatatc atttgtattt ccacaaagca
9721 tttctattgg ttctttagta attacctaaa gagacgtgta gtctttaatg gtgtttcctt
9781 tagtactttt gaagaagctg cgctgtgcac ctttttgtta aataaagaaa tgtatctaaa
9841 gttgcgtagt gatgtgctat tacctcttac gcaatataat agatacttag ctctttataa
9901 taagtacaag tattttagtg gagcaatgga tacaactagc tacagagaag ctgcttgttg
9961 tcatctcgca aaggctctca atgacttcag taactcaggt tctgatgttc tttaccaacc
10021 accacaaacc tctatcacct cagctgtttt gcagagtggt tttagaaaaa tggcattccc
10081 atctggtaaa gttgagggtt gtatggtaca agtaacttgt ggtacaacta cacttaacgg
10141 tctttggctt gatgacgtag tttactgtcc aagacatgtg atctgcacct ctgaagacat
10201 gcttaaccct aattatgaag atttactcat tcgtaagtct aatcataatt tcttggtaca
10261 ggctggtaat gttcaactca gggttattgg acattctatg caaaattgtg tacttaagct
10321 taaggttgat acagccaatc ctaagacacc taagtataag tttgttcgca ttcaaccagg
10381 acagactttt tcagtgttag cttgttacaa tggttcacca tctggtgttt accaatgtgc
10441 tatgaggccc aatttcacta ttaagggttc attccttaat ggttcatgtg gtagtgttgg
10501 ttttaacata gattatgact gtgtctcttt ttgttacatg caccatatgg aattaccaac
10561 tggagttcat gctggcacag acttagaagg taacttttat ggaccttttg ttgacaggca
10621 aacagcacaa gcagctggta cggacacaac tattacagtt aatgttttag cttggttgta
10681 cgctgctgtt ataaatggag acaggtggtt tctcaatcga tttaccacaa ctcttaatga
10741 ctttaacctt gtggctatga agtacaatta tgaacctcta acacaagacc atgttgacat
10801 actaggacct ctttctgctc aaactggaat tgccgtttta gatatgtgtg cttcattaaa
10861 agaattactg caaaatggta tgaatggacg taccatattg ggtagtgctt tattagaaga
10921 tgaatttaca ccttttgatg ttgttagaca atgctcaggt gttactttcc aaagtgcagt
10981 gaaaagaaca atcaagggta cacaccactg gttgttactc acaattttga cttcactttt
11041 agttttagtc cagagtactc aatggtcttt gttctttttt ttgtatgaaa atgccttttt
11101 accttttgct atgggtatta ttgctatgtc tgcttttgca atgatgtttg tcaaacataa
11161 gcatgcattt ctctgtttgt ttttgttacc ttctcttgcc actgtagctt attttaatat
11221 ggtctatatg cctgctagtt gggtgatgcg tattatgaca tggttggata tggttgatac
11281 tagtttgtct ggttttaagc taaaagactg tgttatgtat gcatcagctg tagtgttact
11341 aatccttatg acagcaagaa ctgtgtatga tgatggtgct aggagagtgt ggacacttat
11401 gaatgtcttg acactcgttt ataaagttta ttatggtaat gctttagatc aagccatttc
11461 catgtgggct cttataatct ctgttacttc taactactca ggtgtagtta caactgtcat
11521 gtttttggcc agaggtattg tttttatgtg tgttgagtat tgccctattt tcttcataac
11581 tggtaataca cttcagtgta taatgctagt ttattgtttc ttaggctatt tttgtacttg
11641 ttactttggc ctcttttgtt tactcaaccg ctactttaga ctgactcttg gtgtttatga
11701 ttacttagtt tctacacagg agtttagata tatgaattca cagggactac tcccacccaa
11761 gaatagcata gatgccttca aactcaacat taaattgttg ggtgttggtg gcaaaccttg
11821 tatcaaagta gccactgtac agtctaaaat gtcagatgta aagtgcacat cagtagtctt
11881 actctcagtt ttgcaacaac tcagagtaga atcatcatct aaattgtggg ctcaatgtgt
11941 ccagttacac aatgacattc tcttagctaa agatactact gaagcctttg aaaaaatggt
12001 ttcactactt tctgttttgc tttccatgca gggtgctgta gacataaaca agctttgtga
12061 agaaatgctg gacaacaggg caaccttaca agctatagcc tcagagttta gttcccttcc
12121 atcatatgca gcttttgcta ctgctcaaga agcttatgag caggctgttg ctaatggtga
12181 ttctgaagtt gttcttaaaa agttgaagaa gtctttgaat gtggctaaat ctgaatttga
12241 ccgtgatgca gccatgcaac gtaagttgga aaagatggct gatcaagcta tgacccaaat
12301 gtataaacag gctagatctg aggacaagag ggcaaaagtt actagtgcta tgcagacaat
12361 gcttttcact atgcttagaa agttggataa tgatgcactc aacaacatta tcaacaatgc
12421 aagagatggt tgtgttccct tgaacataat acctcttaca acagcagcca aactaatggt
12481 tgtcatacca gactataaca catataaaaa tacgtgtgat ggtacaacat ttacttatgc
12541 atcagcattg tgggaaatcc aacaggttgt agatgcagat agtaaaattg ttcaacttag
12601 tgaaattagt atggacaatt cacctaattt agcatggcct cttattgtaa cagctttaag
12661 ggccaattct gctgtcaaat tacagaataa tgagcttagt cctgttgcac tacgacagat
12721 gtcttgtgct gccggtacta cacaaactgc ttgcactgat gacaatgcgt tagcttacta
12781 caacacaaca aagggaggta ggtttgtact tgcactgtta tccgatttac aggatttgaa
12841 atgggctaga ttccctaaga gtgatggaac tggtactatc tatacagaac tggaaccacc
12901 ttgtaggttt gttacagaca cacctaaagg tcctaaagtg aagtatttat actttattaa
12961 aggattaaac aacctaaata gaggtatggt acttggtagt ttagctgcca cagtacgtct
13021 acaagctggt aatgcaacag aagtgcctgc caattcaact gtattatctt tctgtgcttt
13081 tgctgtagat gctgctaaag cttacaaaga ttatctagct agtgggggac aaccaatcac
13141 taattgtgtt aagatgttgt gtacacacac tggtactggt caggcaataa cagttacacc
13201 ggaagccaat atggatcaag aatcctttgg tggtgcatcg tgttgtctgt actgccgttg
13261 ccacatagat catccaaatc ctaaaggatt ttgtgactta aaaggtaagt atgtacaaat
13321 acctacaact tgtgctaatg accctgtggg ttttacactt aaaaacacag tctgtaccgt
13381 ctgcggtatg tggaaaggtt atggctgtag ttgtgatcaa ctccgcgaac ccatgcttca
13441 gtcagctgat gcacaatcgt ttttaaacgg gtttgcggtg taagtgcagc ccgtcttaca
13501 ccgtgcggca caggcactag tactgatgtc gtatacaggg cttttgacat ctacaatgat
13561 aaagtagctg gttttgctaa attcctaaaa actaattgtt gtcgcttcca agaaaaggac
13621 gaagatgaca atttaattga ttcttacttt gtagttaaga gacacacttt ctctaactac
13681 caacatgaag aaacaattta taatttactt aaggattgtc cagctgttgc taaacatgac
13741 ttctttaagt ttagaataga cggtgacatg gtaccacata tatcacgtca acgtcttact
13801 aaatacacaa tggcagacct cgtctatgct ttaaggcatt ttgatgaagg taattgtgac
13861 acattaaaag aaatacttgt cacatacaat tgttgtgatg atgattattt caataaaaag
13921 gactggtatg attttgtaga aaacccagat atattacgcg tatacgccaa cttaggtgaa
13981 cgtgtacgcc aagctttgtt aaaaacagta caattctgtg atgccatgcg aaatgctggt
14041 attgttggtg tactgacatt agataatcaa gatctcaatg gtaactggta tgatttcggt
14101 gatttcatac aaaccacgcc aggtagtgga gttcctgttg tagattctta ttattcattg
14161 ttaatgccta tattaacctt gaccagggct ttaactgcag agtcacatgt tgacactgac
14221 ttaacaaagc cttacattaa gtgggatttg ttaaaatatg acttcacgga agagaggtta
14281 aaactctttg accgttattt taaatattgg gatcagacat accacccaaa ttgtgttaac
14341 tgtttggatg acagatgcat tctgcattgt gcaaacttta atgttttatt ctctacagtg
14401 ttcccaccta caagttttgg accactagtg agaaaaatat ttgttgatgg tgttccattt
14461 gtagtttcaa ctggatacca cttcagagag ctaggtgttg tacataatca ggatgtaaac
14521 ttacatagct ctagacttag ttttaaggaa ttacttgtgt atgctgctga ccctgctatg
14581 cacgctgctt ctggtaatct attactagat aaacgcacta cgtgcttttc agtagctgca
14641 cttactaaca atgttgcttt tcaaactgtc aaacccggta attttaacaa agacttctat
14701 gactttgctg tgtctaaggg tttctttaag gaaggaagtt ctgttgaatt aaaacacttc
14761 ttctttgctc aggatggtaa tgctgctatc agcgattatg actactatcg ttataatcta
14821 ccaacaatgt gtgatatcag acaactacta tttgtagttg aagttgttga taagtacttt
14881 gattgttacg atggtggctg tattaatgct aaccaagtca tcgtcaacaa cctagacaaa
14941 tcagctggtt ttccatttaa taaatggggt aaggctagac tttattatga ttcaatgagt
15001 tatgaggatc aagatgcact tttcgcatat acaaaacgta atgtcatccc tactataact
15061 caaatgaatc ttaagtatgc cattagtgca aagaatagag ctcgcaccgt agctggtgtc
15121 tctatctgta gtactatgac caatagacag tttcatcaaa aattattgaa atcaatagcc
15181 gccactagag gagctactgt agtaattgga acaagcaaat tctatggtgg ttggcacaac
15241 atgttaaaaa ctgtttatag tgatgtagaa aaccctcacc ttatgggttg ggattatcct
15301 aaatgtgata gagccatgcc taacatgctt agaattatgg cctcacttgt tcttgctcgc
15361 aaacatacaa cgtgttgtag cttgtcacac cgtttctata gattagctaa tgagtgtgct
15421 caagtattga gtgaaatggt catgtgtggc ggttcactat atgttaaacc aggtggaacc
15481 tcatcaggag atgccacaac tgcttatgct aatagtgttt ttaacatttg tcaagctgtc
15541 acggccaatg ttaatgcact tttatctact gatggtaaca aaattgccga taagtatgtc
15601 cgcaatttac aacacagact ttatgagtgt ctctatagaa atagagatgt tgacacagac
15661 tttgtgaatg agttttacgc atatttgcgt aaacatttct caatgatgat actctctgac
15721 gatgctgttg tgtgtttcaa tagcacttat gcatctcaag gtctagtggc tagcataaag
15781 aactttaagt cagttcttta ttatcaaaac aatgttttta tgtctgaagc aaaatgttgg
15841 actgagactg accttactaa aggacctcat gaattttgct ctcaacatac aatgctagtt
15901 aaacagggtg atgattatgt gtaccttcct tacccagatc catcaagaat cctaggggcc
15961 ggctgttttg tagatgatat cgtaaaaaca gatggtacac ttatgattga acggttcgtg
16021 tctttagcta tagatgctta cccacttact aaacatccta atcaggagta tgctgatgtc
16081 tttcatttgt acttacaata cataagaaag ctacatgatg agttaacagg acacatgtta
16141 gacatgtatt ctgttatgct tactaatgat aacacttcaa ggtattggga acctgagttt
16201 tatgaggcta tgtacacacc gcatacagtc ttacaggctg ttggggcttg tgttctttgc
16261 aattcacaga cttcattaag atgtggtgct tgcatacgta gaccattctt atgttgtaaa
16321 tgctgttacg accatgtcat atcaacatca cataaattag tcttgtctgt taatccgtat
16381 gtttgcaatg ctccaggttg tgatgtcaca gatgtgactc aactttactt aggaggtatg
16441 agctattatt gtaaatcaca taaaccaccc attagttttc cattgtgtgc taatggacaa
16501 gtttttggtt tatataaaaa tacatgtgtt ggtagcgata atgttactga ctttaatgca
16561 attgcaacat gtgactggac aaatgctggt gattacattt tagctaacac ctgtactgaa
16621 agactcaagc tttttgcagc agaaacgctc aaagctactg aggagacatt taaactgtct
16681 tatggtattg ctactgtacg tgaagtgctg tctgacagag aattacatct ttcatgggaa
16741 gttggtaaac ctagaccacc acttaaccga aattatgtct ttactggtta tcgtgtaact
16801 aaaaacagta aagtacaaat aggagagtac acctttgaaa aaggtgacta tggtgatgct
16861 gttgtttacc gaggtacaac aacttacaaa ttaaatgttg gtgattattt tgtgctgaca
16921 tcacatacag taatgccatt aagtgcacct acactagtgc cacaagagca ctatgttaga
16981 attactggct tatacccaac actcaatatc tcagatgagt tttctagcaa tgttgcaaat
17041 tatcaaaagg ttggtatgca aaagtattct acactccagg gaccacctgg tactggtaag
17101 agtcattttg ctattggcct agctctctac tacccttctg ctcgcatagt gtatacagct
17161 tgctctcatg ccgctgttga tgcactatgt gagaaggcat taaaatattt gcctatagat
17221 aaatgtagta gaattatacc tgcacgtgct cgtgtagagt gttttgataa attcaaagtg
17281 aattcaacat tagaacagta tgtcttttgt actgtaaatg cattgcctga gacgacagca
17341 gatatagttg tctttgatga aatttcaatg gccacaaatt atgatttgag tgttgtcaat
17401 gccagattac gtgctaagca ctatgtgtac attggcgacc ctgctcaatt acctgcacca
17461 cgcacattgc taactaaggg cacactagaa ccagaatatt tcaattcagt gtgtagactt
17521 atgaaaacta taggtccaga catgttcctc ggaacttgtc ggcgttgtcc tgctgaaatt
17581 gttgacactg tgagtgcttt ggtttatgat aataagctta aagcacataa agacaaatca
17641 gctcaatgct ttaaaatgtt ttataagggt gttatcacgc atgatgtttc atctgcaatt
17701 aacaggccac aaataggcgt ggtaagagaa ttccttacac gtaaccctgc ttggagaaaa
17761 gctgtcttta tttcacctta taattcacag aatgctgtag cctcaaagat tttgggacta
17821 ccaactcaaa ctgttgattc atcacagggc tcagaatatg actatgtcat attcactcaa
17881 accactgaaa cagctcactc ttgtaatgta aacagattta atgttgctat taccagagca
17941 aaagtaggca tactttgcat aatgtctgat agagaccttt atgacaagtt gcaatttaca
18001 agtcttgaaa ttccacgtag gaatgtggca actttacaag ctgaaaatgt aacaggactc
18061 tttaaagatt gtagtaaggt aatcactggg ttacatccta cacaggcacc tacacacctc
18121 agtgttgaca ctaaattcaa aactgaaggt ttatgtgttg acatacctgg catacctaag
18181 gacatgacct atagaagact catctctatg atgggtttta aaatgaatta tcaagttaat
18241 ggttacccta acatgtttat cacccgcgaa gaagctataa gacatgtacg tgcatggatt
18301 ggcttcgatg tcgaggggtg tcatgctact agagaagctg ttggtaccaa tttaccttta
18361 cagctaggtt tttctacagg tgttaaccta gttgctgtac ctacaggtta tgttgataca
18421 cctaataata cagatttttc cagagttagt gctaaaccac cgcctggaga tcaatttaaa
18481 cacctcatac cacttatgta caaaggactt ccttggaatg tagtgcgtat aaagattgta
18541 caaatgttaa gtgacacact taaaaatctc tctgacagag tcgtatttgt cttatgggca
18601 catggctttg agttgacatc tatgaagtat tttgtgaaaa taggacctga gcgcacctgt
18661 tgtctatgtg atagacgtgc cacatgcttt tccactgctt cagacactta tgcctgttgg
18721 catcattcta ttggatttga ttacgtctat aatccgttta tgattgatgt tcaacaatgg
18781 ggttttacag gtaacctaca aagcaaccat gatctgtatt gtcaagtcca tggtaatgca
18841 catgtagcta gttgtgatgc aatcatgact aggtgtctag ctgtccacga gtgctttgtt
18901 aagcgtgttg actggactat tgaatatcct ataattggtg atgaactgaa gattaatgcg
18961 gcttgtagaa aggttcaaca catggttgtt aaagctgcat tattagcaga caaattccca
19021 gttcttcacg acattggtaa ccctaaagct attaagtgtg tacctcaagc tgatgtagaa
19081 tggaagttct atgatgcaca gccttgtagt gacaaagctt ataaaataga agaattattc
19141 tattcttatg ccacacattc tgacaaattc acagatggtg tatgcctatt ttggaattgc
19201 aatgtcgata gatatcctgc taattccatt gtttgtagat ttgacactag agtgctatct
19261 aaccttaact tgcctggttg tgatggtggc agtttgtatg taaataaaca tgcattccac
19321 acaccagctt ttgataaaag tgcttttgtt aatttaaaac aattaccatt tttctattac
19381 tctgacagtc catgtgagtc tcatggaaaa caagtagtgt cagatataga ttatgtacca
19441 ctaaagtctg ctacgtgtat aacacgttgc aatttaggtg gtgctgtctg tagacatcat
19501 gctaatgagt acagattgta tctcgatgct tataacatga tgatctcagc tggctttagc
19561 ttgtgggttt acaaacaatt tgatacttat aacctctgga acacttttac aagacttcag
19621 agtttagaaa atgtggcttt taatgttgta aataagggac actttgatgg acaacagggt
19681 gaagtaccag tttctatcat taataacact gtttacacaa aagttgatgg tgttgatgta
19741 gaattgtttg aaaataaaac aacattacct gttaatgtag catttgagct ttgggctaag
19801 cgcaacatta aaccagtacc agaggtgaaa atactcaata atttgggtgt ggacattgct
19861 gctaatactg tgatctggga ctacaaaaga gatgctccag cacatatatc tactattggt
19921 gtttgttcta tgactgacat agccaagaaa ccaactgaaa cgatttgtgc accactcact
19981 gtcttttttg atggtagagt tgatggtcaa gtagacttat ttagaaatgc ccgtaatggt
20041 gttcttatta cagaaggtag tgttaaaggt ttacaaccat ctgtaggtcc caaacaagct
20101 agtcttaatg gagtcacatt aattggagaa gccgtaaaaa cacagttcaa ttattataag
20161 aaagttgatg gtgttgtcca acaattacct gaaacttact ttactcagag tagaaattta
20221 caagaattta aacccaggag tcaaatggaa attgatttct tagaattagc tatggatgaa
20281 ttcattgaac ggtataaatt agaaggctat gccttcgaac atatcgttta tggagatttt
20341 agtcatagtc agttaggtgg tttacatcta ctgattggac tagctaaacg ttttaaggaa
20401 tcaccttttg aattagaaga ttttattcct atggacagta cagttaaaaa ctatttcata
20461 acagatgcgc aaacaggttc atctaagtgt gtgtgttctg ttattgattt attacttgat
20521 gattttgttg aaataataaa atcccaagat ttatctgtag tttctaaggt tgtcaaagtg
20581 actattgact atacagaaat ttcatttatg ctttggtgta aagatggcca tgtagaaaca
20641 ttttacccaa aattacaatc tagtcaagcg tggcaaccgg gtgttgctat gcctaatctt
20701 tacaaaatgc aaagaatgct attagaaaag tgtgaccttc aaaattatgg tgatagtgca
20761 acattaccta aaggcataat gatgaatgtc gcaaaatata ctcaactgtg tcaatattta
20821 aacacattaa cattagctgt accctataat atgagagtta tacattttgg tgctggttct
20881 gataaaggag ttgcaccagg tacagctgtt ttaagacagt ggttgcctac gggtacgctg
20941 cttgtcgatt cagatcttaa tgactttgtc tctgatgcag attcaacttt gattggtgat
21001 tgtgcaactg tacatacagc taataaatgg gatctcatta ttagtgatat gtacgaccct
21061 aagactaaaa atgttacaaa agaaaatgac tctaaagagg gttttttcac ttacatttgt
21121 gggtttatac aacaaaagct agctcttgga ggttccgtgg ctataaagat aacagaacat
21181 tcttggaatg ctgatcttta taagctcatg ggacacttcg catggtggac agcctttgtt
21241 actaatgtga atgcgtcatc atctgaagca tttttaattg gatgtaatta tcttggcaaa
21301 ccacgcgaac aaatagatgg ttatgtcatg catgcaaatt acatattttg gaggaataca
21361 aatccaattc agttgtcttc ctattcttta tttgacatga gtaaatttcc ccttaaatta
21421 aggggtactg ctgttatgtc tttaaaagaa ggtcaaatca atgatatgat tttatctctt
21481 cttagtaaag gtagacttat aattagagaa aacaacagag ttgttatttc tagtgatgtt
21541 cttgttaaca actaaacgaa caatgtttgt ttttcttgtt ttattgccac tagtctctag
21601 tcagtgtgtt aatcttacaa ccagaactca attaccccct gcatacacta attctttcac
21661 acgtggtgtt tattaccctg acaaagtttt cagatcctca gttttacatt caactcagga
21721 cttgttctta cctttctttt ccaatgttac ttggttccat gctatacatg tctctgggac
21781 caatggtact aagaggtttg ataaccctgt cctaccattt aatgatggtg tttattttgc
21841 ttccactgag aagtctaaca taataagagg ctggattttt ggtactactt tagattcgaa
21901 gacccagtcc ctacttattg ttaataacgc tactaatgtt gttattaaag tctgtgaatt
21961 tcaattttgt aatgatccat ttttgggtgt ttattaccac aaaaacaaca aaagttggat
22021 ggaaagtgag ttcagagttt attctagtgc gaataattgc acttttgaat atgtctctca
22081 gccttttctt atggaccttg aaggaaaaca gggtaatttc aaaaatctta gggaatttgt
22141 gtttaagaat attgatggtt attttaaaat atattctaag cacacgccta ttaatttagt
22201 gcgtgatctc cctcagggtt tttcggcttt agaaccattg gtagatttgc caataggtat
22261 taacatcact aggtttcaaa ctttacttgc tttacataga agttatttga ctcctggtga
22321 ttcttcttca ggttggacag ctggtgctgc agcttattat gtgggttatc ttcaacctag
22381 gacttttcta ttaaaatata atgaaaatgg aaccattaca gatgctgtag actgtgcact
22441 tgaccctctc tcagaaacaa agtgtacgtt gaaatccttc actgtagaaa aaggaatcta
22501 tcaaacttct aactttagag tccaaccaac agaatctatt gttagatttc ctaatattac
22561 aaacttgtgc ccttttggtg aagtttttaa cgccaccaga tttgcatctg tttatgcttg
22621 gaacaggaag agaatcagca actgtgttgc tgattattct gtcctatata attccgcatc
22681 attttccact tttaagtgtt atggagtgtc tcctactaaa ttaaatgatc tctgctttac
22741 taatgtctat gcagattcat ttgtaattag aggtgatgaa gtcagacaaa tcgctccagg
22801 gcaaactgga aagattgctg attataatta taaattacca gatgatttta caggctgcgt
22861 tatagcttgg aattctaaca atcttgattc taaggttggt ggtaattata attacctgta
22921 tagattgttt aggaagtcta atctcaaacc ttttgagaga gatatttcaa ctgaaatcta
22981 tcaggccggt agcacacctt gtaatggtgt tgaaggtttt aattgttact ttcctttaca
23041 atcatatggt ttccaaccca ctaatggtgt tggttaccaa ccatacagag tagtagtact
23101 ttcttttgaa cttctacatg caccagcaac tgtttgtgga cctaaaaagt ctactaattt
23161 ggttaaaaac aaatgtgtca atttcaactt caatggttta acaggcacag gtgttcttac
23221 tgagtctaac aaaaagtttc tgcctttcca acaatttggc agagacattg ctgacactac
23281 tgatgctgtc cgtgatccac agacacttga gattcttgac attacaccat gttcttttgg
23341 tggtgtcagt gttataacac caggaacaaa tacttctaac caggttgctg ttctttatca
23401 ggatgttaac tgcacagaag tccctgttgc tattcatgca gatcaactta ctcctacttg
23461 gcgtgtttat tctacaggtt ctaatgtttt tcaaacacgt gcaggctgtt taataggggc
23521 tgaacatgtc aacaactcat atgagtgtga catacccatt ggtgcaggta tatgcgctag
23581 ttatcagact cagactaatt ctcctcggcg ggcacgtagt gtagctagtc aatccatcat
23641 tgcctacact atgtcacttg gtgcagaaaa ttcagttgct tactctaata actctattgc
23701 catacccaca aattttacta ttagtgttac cacagaaatt ctaccagtgt ctatgaccaa
23761 gacatcagta gattgtacaa tgtacatttg tggtgattca actgaatgca gcaatctttt
23821 gttgcaatat ggcagttttt gtacacaatt aaaccgtgct ttaactggaa tagctgttga
23881 acaagacaaa aacacccaag aagtttttgc acaagtcaaa caaatttaca aaacaccacc
23941 aattaaagat tttggtggtt ttaatttttc acaaatatta ccagatccat caaaaccaag
24001 caagaggtca tttattgaag atctactttt caacaaagtg acacttgcag atgctggctt
24061 catcaaacaa tatggtgatt gccttggtga tattgctgct agagacctca tttgtgcaca
24121 aaagtttaac ggccttactg ttttgccacc tttgctcaca gatgaaatga ttgctcaata
24181 cacttctgca ctgttagcgg gtacaatcac ttctggttgg acctttggtg caggtgctgc
24241 attacaaata ccatttgcta tgcaaatggc ttataggttt aatggtattg gagttacaca
24301 gaatgttctc tatgagaacc aaaaattgat tgccaaccaa tttaatagtg ctattggcaa
24361 aattcaagac tcactttctt ccacagcaag tgcacttgga aaacttcaag atgtggtcaa
24421 ccaaaatgca caagctttaa acacgcttgt taaacaactt agctccaatt ttggtgcaat
24481 ttcaagtgtt ttaaatgata tcctttcacg tcttgacaaa gttgaggctg aagtgcaaat
24541 tgataggttg atcacaggca gacttcaaag tttgcagaca tatgtgactc aacaattaat
24601 tagagctgca gaaatcagag cttctgctaa tcttgctgct actaaaatgt cagagtgtgt
24661 acttggacaa tcaaaaagag ttgatttttg tggaaagggc tatcatctta tgtccttccc
24721 tcagtcagca cctcatggtg tagtcttctt gcatgtgact tatgtccctg cacaagaaaa
24781 gaacttcaca actgctcctg ccatttgtca tgatggaaaa gcacactttc ctcgtgaagg
24841 tgtctttgtt tcaaatggca cacactggtt tgtaacacaa aggaattttt atgaaccaca
24901 aatcattact acagacaaca catttgtgtc tggtaactgt gatgttgtaa taggaattgt
24961 caacaacaca gtttatgatc ctttgcaacc tgaattagac tcattcaagg aggagttaga
25021 taaatatttt aagaatcata catcaccaga tgttgattta ggtgacatct ctggcattaa
25081 tgcttcagtt gtaaacattc aaaaagaaat tgaccgcctc aatgaggttg ccaagaattt
25141 aaatgaatct ctcatcgatc tccaagaact tggaaagtat gagcagtata taaaatggcc
25201 atggtacatt tggctaggtt ttatagctgg cttgattgcc atagtaatgg tgacaattat
25261 gctttgctgt atgaccagtt gctgtagttg tctcaagggc tgttgttctt gtggatcctg
25321 ctgcaaattt gatgaagacg actctgagcc agtgctcaaa ggagtcaaat tacattacac
25381 ataaacgaac ttatggattt gtttatgaga atcttcacaa ttggaactgt aactttgaag
25441 caaggtgaaa tcaaggatgc tactccttca gattttgttc gcgctactgc aacgataccg
25501 atacaagcct cactcccttt cggatggctt attgttggcg ttgcacttct tgctgttttt
25561 cagagcgctt ccaaaatcat aaccctcaaa aagagatggc aactagcact ctccaagggt
25621 gttcactttg tttgcaactt gctgttgttg tttgtaacag tttactcaca ccttttgctc
25681 gttgctgctg gccttgaagc cccttttctc tatctttatg ctttagtcta cttcttgcag
25741 agtataaact ttgtaagaat aataatgagg ctttggcttt gctggaaatg ccgttccaaa
25801 aacccattac tttatgatgc caactatttt ctttgctggc atactaattg ttacgactat
25861 tgtatacctt acaatagtgt aacttcttca attgtcatta cttcaggtga tggcacaaca
25921 agtcctattt ctgaacatga ctaccagatt ggtggttata ctgaaaaatg ggaatctgga
25981 gtaaaagact gtgttgtatt acacagttac ttcacttcag actattacca gctgtactca
26041 actcaattga gtacagacac tggtgttgaa catgttacct tcttcatcta caataaaatt
26101 gttgatgagc ctgaagaaca tgtccaaatt cacacaatcg acggttcatc cggagttgtt
26161 aatccagtaa tggaaccaat ttatgatgaa ccgacgacga ctactagcgt gcctttgtaa
26221 gcacaagctg atgagtacga acttatgtac tcattcgttt cggaagagac aggtacgtta
26281 atagttaata gcgtacttct ttttcttgct ttcgtggtat tcttgctagt tacactagcc
26341 atccttactg cgcttcgatt gtgtgcgtac tgctgcaata ttgttaacgt gagtcttgta
26401 aaaccttctt tttacgttta ctctcgtgtt aaaaatctga attcttctag agttcctgat
26461 cttctggtct aaacgaacta aatattatat tagtttttct gtttggaact ttaattttag
26521 ccatggcaga ttccaacggt actattaccg ttgaagagct taaaaagctc cttgaacaat
26581 ggaacctagt aataggtttc ctattcctta catggatttg tcttctacaa tttgcctatg
26641 ccaacaggaa taggtttttg tatataatta agttaatttt cctctggctg ttatggccag
26701 taactttagc ttgttttgtg cttgctgctg tttacagaat aaattggatc accggtggaa
26761 ttgctatcgc aatggcttgt cttgtaggct tgatgtggct cagctacttc attgcttctt
26821 tcagactgtt tgcgcgtacg cgttccatgt ggtcattcaa tccagaaact aacattcttc
26881 tcaacgtgcc actccatggc actattctga ccagaccgct tctagaaagt gaactcgtaa
26941 tcggagctgt gatccttcgt ggacatcttc gtattgctgg acaccatcta ggacgctgtg
27001 acatcaagga cctgcctaaa gaaatcactg ttgctacatc acgaacgctt tcttattaca
27061 aattgggagc ttcgcagcgt gtagcaggtg actcaggttt tgctgcatac agtcgctaca
27121 ggattggcaa ctataaatta aacacagacc attccagtag cagtgacaat attgctttgc
27181 ttgtacagta agtgacaaca gatgtttcat ctcgttgact ttcaggttac tatagcagag
27241 atattactaa ttattatgag gacttttaaa gtttccattt ggaatcttga ttacatcata
27301 aacctcataa ttaaaaattt atctaagtca ctaactgaga ataaatattc tcaattagat
27361 gaagagcaac caatggagat tgattaaacg aacatgaaaa ttattctttt cttggcactg
27421 ataacactcg ctacttgtga gctttatcac taccaagagt gtgttagagg tacaacagta
27481 cttttaaaag aaccttgctc ttctggaaca tacgagggca attcaccatt tcatcctcta
27541 gctgataaca aatttgcact gacttgcttt agcactcaat ttgcttttgc ttgtcctgac
27601 ggcgtaaaac acgtctatca gttacgtgcc agatcagttt cacctaaact gttcatcaga
27661 caagaggaag ttcaagaact ttactctcca atttttctta ttgttgcggc aatagtgttt
27721 ataacacttt gcttcacact caaaagaaag acagaatgat tgaactttca ttaattgact
27781 tctatttgtg ctttttagcc tttctgctat tccttgtttt aattatgctt attatctttt
27841 ggttctcact tgaactgcaa gatcataatg aaacttgtca cgcctaaacg aacatgaaat
27901 ttcttgtttt cttaggaatc atcacaactg tagctgcatt tcaccaagaa tgtagtttac
27961 agtcatgtac tcaacatcaa ccatatgtag ttgatgaccc gtgtcctatt cacttctatt
28021 ctaaatggta tattagagta ggagctagaa aatcagcacc tttaattgaa ttgtgcgtgg
28081 atgaggctgg ttctaaatca cccattcagt acatcgatat cggtaattat acagtttcct
28141 gtttaccttt tacaattaat tgccaggaac ctaaattggg tagtcttgta gtgcgttgtt
28201 cgttctatga agacttttta gagtatcatg acgttcgtgt tgttttagat ttcatctaaa
28261 cgaacaaact aaaatgtctg ataatggacc ccaaaatcag cgaaatgcac cccgcattac
28321 gtttggtgga ccctcagatt caactggcag taaccagaat ggagaacgca gtggggcgcg
28381 atcaaaacaa cgtcggcccc aaggtttacc caataatact gcgtcttggt tcaccgctct
28441 cactcaacat ggcaaggaag accttaaatt ccctcgagga caaggcgttc caattaacac
28501 caatagcagt ccagatgacc aaattggcta ctaccgaaga gctaccagac gaattcgtgg
28561 tggtgacggt aaaatgaaag atctcagtcc aagatggtat ttctactacc taggaactgg
28621 gccagaagct ggacttccct atggtgctaa caaagacggc atcatatggg ttgcaactga
28681 gggagccttg aatacaccaa aagatcacat tggcacccgc aatcctgcta acaatgctgc
28741 aatcgtgcta caacttcctc aaggaacaac attgccaaaa ggcttctacg cagaagggag
28801 cagaggcggc agtcaagcct cttctcgttc ctcatcacgt agtcgcaaca gttcaagaaa
28861 ttcaactcca ggcagcagta ggggaacttc tcctgctaga atggctggca atggcggtga
28921 tgctgctctt gctttgctgc tgcttgacag attgaaccag cttgagagca aaatgtctgg
28981 taaaggccaa caacaacaag gccaaactgt cactaagaaa tctgctgctg aggcttctaa
29041 gaagcctcgg caaaaacgta ctgccactaa agcatacaat gtaacacaag ctttcggcag
29101 acgtggtcca gaacaaaccc aaggaaattt tggggaccag gaactaatca gacaaggaac
29161 tgattacaaa cattggccgc aaattgcaca atttgccccc agcgcttcag cgttcttcgg
29221 aatgtcgcgc attggcatgg aagtcacacc ttcgggaacg tggttgacct acacaggtgc
29281 catcaaattg gatgacaaag atccaaattt caaagatcaa gtcattttgc tgaataagca
29341 tattgacgca tacaaaacat tcccaccaac agagcctaaa aaggacaaaa agaagaaggc
29401 tgatgaaact caagccttac cgcagagaca gaagaaacag caaactgtga ctcttcttcc
29461 tgctgcagat ttggatgatt tctccaaaca attgcaacaa tccatgagca gtgctgactc
29521 aactcaggcc taaactcatg cagaccacac aaggcagatg ggctatataa acgttttcgc
29581 ttttccgttt acgatatata gtctactctt gtgcagaatg aattctcgta actacatagc
29641 acaagtagat gtagttaact ttaatctcac atagcaatct ttaatcagtg tgtaacatta
29701 gggaggactt gaaagagcca ccacattttc accgaggcca cgcggagtac gatcgagtgt
29761 acagtgaaca atgctaggga gagctgccta tatggaagag ccctaatgtg taaaattaat
29821 tttagtagtg ctatccccat gtgattttaa tagcttctta ggagaatgac aaaaaaaaaa
29881 aaaaaaaaaa aaaaaaaaaa aaa

TGC TGT TGA GGA GCT GGA (SEQ ID NO:2)

AGC ACA CCA GGC AGA GTT (SEQ ID NO:3).

CGG CTG AGG AGC GGA AGA (SEQ ID NO:4)

TGG-AGG-TGA-GCA-ATC-CCC (SEQ ID NO:5)

TTAATACGACTCACTATAGG (SEQ ID NO:6)

GTTTTAGAGCTAGAAATAGC (SEQ ID NO:7)

TGAGAAGCGACAGCCAGTGA (SEQ ID NO:8)

GGCCAGACTGGCACCAACGG (SEQ ID NO:9)

GGACTCCAAAGAATCATTAACTCCTGG (SEQ ID NO:10)

AATTACCCCAGGGGTGCAGA (SEQ ID NO:11)

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. An ex vivo method of preparing SARS-CoV-2-specific T cells, comprising the step of culturing a starting population of cells in the presence of a mixture of peptides and one or more of IL-2, IL-4, IL-7, IL-15, and IL-21, wherein the mixture of peptides comprises overlapping peptides spanning at least one protein from SARS-CoV-2, thereby producing SARS-CoV-2-specific T cells.

2. The method of claim 1, wherein the SARS-CoV-2-specific T cells are further defined as cytotoxic T cells (CTLs).

3. The method of claim 1 or 2, wherein the starting population of cells comprises peripheral blood mononuclear cells (PBMCs), lymphocytes, or a mixture thereof.

4. The method of any one of claims 1-3, wherein the starting population is derived from one or more healthy donors, one or more individuals that will receive the cells, one or more asymptomatic SARS-CoV-2-positive individuals, one or more SARS-CoV-2-negative individuals, one or more individuals that were SARS-CoV-2-positive followed by being SARS-CoV-2-negative, one or more individuals that have natural or acquired antibodies to SARS-CoV-2, one or more individuals that lack antibodies to SARS-CoV-2, one or more individuals previously vaccinated for SARS-CoV-2, or a combination thereof.

5. The method of claim 3 or 4, wherein the mononuclear cells were obtained from blood, buffy coat, or both.

6. The method of any one of claims 1-5, wherein the culturing is for 7-14 days.

7. The method of any one of claims 1-6, wherein the culturing is for 10-14 days.

8. The method of any one of claims 1-7, wherein the culturing does not comprise a second population of cells pre-stimulated with the mixture of peptides.

9. The method of claim 8, wherein the pre-stimulated cells are further defined as antigen presenting cells (APCs).

10. The method of claim 9, wherein the APCs are dendritic cells, monocytes, and/or B lymphocytes.

11. The method of any one of claims 1-10, wherein the peptides have a length of 8-30 amino acids.

12. The method of any one of claims 1-11, wherein the peptides have a length of 12-18 amino acids.

13. The method of any one of claims 1-12, wherein the peptides are 15 amino acids in length.

14. The method of any one of claims 1-13, wherein peptides in the mixture of peptides overlap by 10-15 contiguous amino acids from a corresponding protein in SARS-CoV-2.

15. The method of claim 14, wherein peptides in the mixture are 15 amino acids in length and overlap by 11 continuous amino acids from a corresponding protein in SARS-CoV-2.

16. The method of any one of claims 1-15, wherein the mixture of peptides comprises peptides that span part or all of the entire length of one or more proteins of SARS-CoV-2.

17. The method of any one of claims 1-15, wherein the mixture of peptides comprises peptides that span all of the entire length of two or more proteins of SARS-CoV-2.

18. The method of any one of claims 1-17, further comprising the step of genetically modifying the starting population of cells or the SARS-CoV-2-specific T cells to
(a) disrupt expression of one or more endogenous genes in the cells; and/or
(b) express one or more chimeric antigen receptors (CAR) and/or one or more T cell receptors (TCR).

19. The method of claim 18, wherein the endogenous gene is one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, TDAG8, Cbl-b, B2M, HLA class I, and CD7.

20. The method of claim 18 or 19 wherein the genetically modifying occurs by CRISPR.

21. The method of any one of claims 18-20, wherein the T cell is genetically modified to express a CAR.

22. The method of any one of claims 18-20, wherein the T cell is genetically modified to express a TCR.

23. The method of any one of claims 18-22, wherein the CAR or TCR targets one or more SARS-CoV-2 antigens.

24. A population of SARS-CoV-2-specific T cells, produced by any one of the methods of claim 1-23.

25. The population of claim 24, comprised in a pharmaceutically acceptable carrier.

26. A composition comprising the population of claim 25.

27. A method of treating or preventing a SARS-CoV-2 infection in an individual, comprising the step of delivering to the individual an effective amount of the population of claim 25 or the composition of claim 26 to the individual.

28. The method of claim 27, wherein the individual has SARS-CoV-2 infection.

29. The method of claim 27, wherein the individual is at high risk for having SARS-CoV-2 infection.

30. The method of claim 27, wherein the individual does not have SARS-CoV-2 infection or has tested negative for SARS-CoV-2 infection.

31. The method of any one of claims 27-30, wherein the cells are autologous or allogeneic with respect to the individual.

32. The method of any one of claims 27-31, wherein the population is deliverd by infusion.

33. The method of any one of claims 27-32, wherein the individual has acute respiratory distress syndrome or pneumonia.

34. The method of any one of claims 27-33, wherein the SARS-CoV-2-specific T cells have been genetically modified to lack expression of or have reduced expression of NR3C1.

35. The method of claim 34, wherein the individual has received, is receiving, and/or will receive one or more glucocorticoids.

36. The method of claim 35, wherein the one or more glucocorticoids is beclomethasone, betamethasone, budesonide cortisone, dexamethasone, hydrocortisone, methylprednisolone, prednisolone, prednisone, triamcinolone, or a combination thereof.

37. The method of any one of claims 27-36, wherein the population is administered by infusion, intravenously, intraperitoneally, intratracheally, intramuscularly, endoscopically, intralesionally, percutaneously, subcutaneously, regionally, intracranially, by direct injection, or by perfusion.

38. The method of any one of claims 27-37, wherein the individual is administered an effective amount of one or more additional therapies.

39. The method of claim 38, wherein the one or more additional therapies are Azithromycin, AC-55541, Apicidin, AZ3451, AZ8838, Bafilomycin A1, CCT 365623, Daunorubicin, E-52862, Entacapone, GB110, H-89, Haloperidol, Indomethacin, JQ1, Loratadine, Merimepodib, Metformin, Midostaurin, Migalastat, Mycophenolic acid, PB28, PD-144418, Ponatinib, Ribavirin, RS-PPCC, Ruxolitinib, RVX-208, S-verapamil, Silmitasertib , TMCB, UCPH-101, Valproic Acid, XL413, ZINC1775962367, ZINC4326719, ZINC4511851, ZINC95559591, 4E2RCat, ABBV-744, Camostat, Captopril, CB5083, Chloramphenicol, Chloroquine (and/or Hydroxychloroquine), CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/M1N128), FK-506 (Tacrolimus), Tematin 4 (DA3), Tigecycline, Tomivosertib (eFT-508), Verdinexor, WDB002, Zotatifin (eFT226), or a combination thereof.

40. The method of claim 38 or 39, wherein the individual is administered an effective amount of SARS-CoV-2-specific NK cells.

41. The method of claim 40, wherein the NK cells comprise:
(a) one or more engineered antigen receptors that target one or more proteins from the SARS-CoV-2 virus;
(b) one or more engineered antigen receptors that target a receptor used by SARS-CoV-2 to enter a host cell; and/or
(c) one or more engineered antigen receptors that target a ligand on the surface of a SARS-CoV-2-infected cell.

42. The method of claim 41, wherein the engineered antigen receptor is a chimeric antigen receptor (CAR), an engineered T cell receptor (TCR), or both.

43. The method of claim 41 or 42, wherein the engineered antigen receptor of (a) comprises at least one scFv that targets a protein from the SARS-CoV-2 virus.

44. The method of any one of claims 41-43, wherein the engineered antigen receptor of (a) targets the spike protein, membrane protein, envelope protein, nucleocapsid protein, Nsp2, Nsp3, Nsp4, Nsp6, Nsp7, Nsp8, Nsp9, Nsp10, Nsp11, 3C-like proteinase, leader protein, ORF7b, 2′-O-ribose methyltransferase, endoRNAse, 3′-to-5′ exonuclease, helicase, RNA-dependent RNA polymerase, orfla polyprotein, ORF10 protein, ORF8 protein, ORF7a protein, ORF6 protein, ORF3a, or orflab polyprotein.

45. The method of any one of claims 41-44, wherein the engineered antigen receptor of (b) targets angiotensin-converting enzyme-2 (ACE2) on a host cell.

46. The method of any one of claims 41-45, wherein the engineered antigen receptor of (c) targets a ligand of Natural killer group 2D receptor (NKG2DR).

47. The method of claim 46, wherein the ligand is MHC class I chain-related protein (MIC)A, MICB, UL16 binding protein 1 (ULBP), or Poliovirus Receptor (PVR).

48. The method of any one of claims 40-47, wherein one or more endogenous genes in the NK cell have been reduced or eliminated in expression.

49. The method of claim 48, wherein the endogenous gene is selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, Cbl-b, B2M, HLA class I, and a combination thereof.

50. The method of any one of claims 1-49, wherein the mixture comprises IL-2, IL-4, and IL-7.

51. The method of any one of claims 1-50, wherein the mixture comprises IL-2, IL-4, and IL-7, but lacks IL-15 and IL-21.


SEQUENCE LISTING (11 sequences)

SEQ ID NO: 1 - DNA, 29903 bp, Severe acute respiratory syndrome coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 18 bp, Artificial Sequence
  Description of Artificial Sequence: Synthetic oligonucleotide
tgctgttgaggagctgga

SEQ ID NO: 3 - DNA, 18 bp, Artificial Sequence
  Description of Artificial Sequence: Synthetic oligonucleotide
agcacaccaggcagagtt

SEQ ID NO: 4 - DNA, 18 bp, Artificial Sequence
  Description of Artificial Sequence: Synthetic oligonucleotide
cggctgaggagcggaaga

SEQ ID NO: 5 - DNA, 18 bp, Artificial Sequence
  Description of Artificial Sequence: Synthetic oligonucleotide
tggaggtgagcaatcccc

SEQ ID NO: 6 - DNA, 20 bp, Artificial Sequence
  Description of Artificial Sequence: Synthetic oligonucleotide
ttaatacgactcactatagg

SEQ ID NO: 7 - DNA, 20 bp, Artificial Sequence
  Description of Artificial Sequence: Synthetic oligonucleotide
gttttagagctagaaatagc

SEQ ID NO: 8 - DNA, 20 bp, Artificial Sequence
  Description of Artificial Sequence: Synthetic oligonucleotide
tgagaagcgacagccagtga

SEQ ID NO: 9 - DNA, 20 bp, Artificial Sequence
  Description of Artificial Sequence: Synthetic oligonucleotide
ggccagactggcaccaacgg

SEQ ID NO: 10 - DNA, 27 bp, Artificial Sequence
  Description of Artificial Sequence: Synthetic primer
ggactccaaagaatcattaactcctgg

SEQ ID NO: 11 - DNA, 20 bp, Artificial Sequence
  Description of Artificial Sequence: Synthetic primer
aattaccccaggggtgcaga